(19) United States
(12) Patent Application Publication
(10) Pub. No.: US 2024/0252223 A1
(43) Pub. Date: Aug. 1, 2024

(54) THERMAL THROMBECTOMY SYSTEMS AND METHODS

(71) Applicant: Koya Medical, Inc., Oakland, CA (US)

(72) Inventors: Anand Doraiswamy, Oakland, CA (US); Matthew Flowers, Laguna Niguel, CA (US); Andrew Schieber, Tustin, CA (US); Reghu Kunnath, Whittier, CA (US); Jarren Baldwin, Oakland, CA (US); Nathan Chu, Irvine, CA (US)

(21) Appl. No.: 18/424,103

(22) Filed: Jan. 26, 2024

Related U.S. Application Data
(60) Provisional application No. 63/520,214, filed on Aug. 17, 2023.
(60) Provisional application No. 63/481,900, filed on Jan. 27, 2023.

(51) Int. Cl. A61B 18/08; A61B 17/00; A61B 18/00

(52) U.S. Cl. CPC A61B 18/082; A61B 2017/00867; A61B 2018/0022; A61B 2018/0041; A61B 2018/0072; A61B 2018/00821; A61B 2018/00827

(57) ABSTRACT

This disclosure relates to thermal thrombectomy systems and methods. The thermal thrombectomy systems can apply heat to a thrombus, softening and/or melting the thrombus, which can include one or more proteins and/or biopolymers of the thrombus, to ease removal. The thermal thrombectomy systems can include a heated wire that can be navigated to the thrombus. The distal portion of the wire can apply heat to the thrombus while the proximal portion may be insulated. The heated wire can include coils and or other features to facilitate engagement with the thrombus.

### CROSS REFERENCE

This application claims the priority benefit of U.S. Provisional Application No. 63/520,214, filed Aug. 17, 2023, and U.S. Provisional Application No. 63/481,900, filed Jan. 27, 2023, which are hereby incorporated by reference in their entireties. All applications for which a foreign or domestic priority is identified in the Application Data Sheet as filed with the present application are hereby incorporated by reference under 37 CFR 1.57.

### FIELD

This disclosure relates to thrombectomy systems and methods.

### BACKGROUND

The vascular system carries blood throughout the body. The vascular system includes arteries that distribute blood containing oxygen from the heart throughout the body and veins that carry deoxygenated blood back to the heart.

Thrombosis occurs when a thrombus (e.g., blood clot) forms within a blood vessel, whether venous or arterial. The thrombus may restrict blood flow through the blood vessel. For example, deep vein thrombosis (DVT) occurs when the thrombus forms in a deep vein, restricting blood flow back toward the heart. DVT typically develops in the lower leg, thigh, or pelvis but may also occur in other locations of the body, such as the arm. DVT can result in swelling, pain, discoloration, scaling, and/or ulcers. Additionally, a fragment (e.g., embolus) of the thrombus may break off and travel through the blood stream to the lungs, resulting in a pulmonary embolism (PE)-a potentially fatal condition.

### SUMMARY

Thrombi can be removed by way of a thrombectomy procedure. A thrombectomy procedure may include navigating a guide wire with the assistance of an imaging system (e.g., fluoroscopic x-ray imaging) through the vascular system to pass through the thrombus to a distal location. A catheter can be advanced along the guide wire and through the thrombus to deploy (e.g., expand) an expandable bag distal of the thrombus. The expandable bag can be retracted proximally, scraping the inner walls of the vessel, to capture the thrombus and deposit it into the catheter for removal. The surgeon performing the thrombectomy may perform multiple passes with the expandable bag to remove the thrombus. Performing multiple passes can be time consuming and frustrating. Additionally, the likelihood of incurring damage to the vessel walls and/or valves increases with each pass. Multiple passes can increase the risk of vessel inflammation and re-thrombosis. Accordingly, thrombectomy solutions to reduce the number of passes to remove a thrombus are needed.

A thrombus can include fibrin, red blood cells, platelets, leukocytes, and neutrophil extracellular traps. As time passes, the thrombus accumulates more collagen and fibrin content. As more collagen is accumulated, the thrombus becomes harder, and it can be difficult for guide wires, catheters, and/or expandable devices to pass through the thrombus. Attempting to pass through a hardened thrombus can be time consuming, frustrating, and/or result in damage to the vessel wall and/or valves.

The thermal thrombectomy systems and methods disclosed herein may at least address the problems indicated above. A thrombus (e.g., one or more proteins and/or biopolymers) may be softened and/or emulsified (e.g., melted, liquefied) by the thermal thrombectomy systems and methods disclosed herein. The softening and/or emulsification of the thrombus can ease penetration with a wire, guide wire, catheter, cutting device, and/or collection device (e.g., expandable device, capture device). The softening or emulsification of the thrombus can enable the thrombus to separate from the vessel walls and endothelial cellular layers more easily. The softening and/or emulsification of the thrombus may help break the thrombus down into fragments more easily extracted with an expandable capture device, such as a balloon, umbrella, basket, bag, scoop, etc., and/or aspiration device. The softening and/or emulsification of the thrombus may enable an expandable device (e.g., capture device) and/or aspiration device to extract the thrombus with a single pass or fewer passes, to facilitate a more efficient and convenient procedure as well as reducing the risk of damage to vessel walls, vessel valves, vessel inflammation, and re-thrombosis. The thermal thrombectomy systems described herein may be used to remove both acute and chronic thrombi in both veins and arteries. For example, the thermal thrombectomy system and methods described herein can insert (e.g., inject) a heated element into a chronic region of a thrombus, which can emulsify the chronic region and/or loosen up the chronic region. The thermal thrombectomy systems and methods disclosed herein may facilitate local removal of thrombi, which may preserve endothelial cells and valves. The thermal thrombectomy systems and methods disclosed herein may remove both chronic and acute thrombi. The thermal thrombectomy systems and methods disclosed herein may access all anatomical blood vessel locations (e.g., below knee and smaller vessels). The thermal thrombectomy systems and methods disclosed herein may reduce blood loss. The thermal thrombectomy systems and methods disclosed herein may avoid systemic thrombolytics. The thermal thrombectomy systems disclosed herein may be disposable (e.g., no console) or reusable.

In some variants, the thermal thrombectomy systems disclosed herein can include a heated wire (e.g., heated element, heated guide wire), which may also be referred to as a temperature controlled wire (e.g., temperature controlled element, temperature controlled guide wire). The heated wire may be heated directly or indirectly with one or more energy sources, which may at least include heat, radio frequency, laser, electricity (e.g., current), resistive heating, inductive heating, ultrasound, hot liquid, nuclear, and/or others. A distal portion of the heated wire may be configured to be disposed at a thrombus to apply heat to the thrombus to soften and/or emulsify the thrombus forming elements thereof. A proximal portion (e.g., the portion configured to be disposed away from the thrombus) may be insulated. The heated wire can be navigated to the thrombus, heated to a temperature sufficient to liquefy or at least soften the thrombus, inserted into thrombus, and advanced through the thrombus to a distal position. Advancing through the thrombus with a heated wire may ease penetration compared to an unheated wire. In some variants, the wire may penetrate the thrombus, which may include passing through to the distal position, prior to heating. In some variants, an expandable device, which may also be heated, can be positioned distal of the thrombus prior to heating of the wire.

In some variants, the thermal thrombectomy systems disclosed herein may include an expandable device (e.g., capture device), which may at least include a bag (e.g., mesh, braded, etc.), inverted mesh, mesh, stent, umbrella, balloon, basket, funnel (e.g., mesh funnel), disc, and/or other device. The expandable device may be advanced along a guide wire, which may be a heated guide wire, to a position distal of the thrombus and deployed (e.g., expanded) to impede fragments, such as emboli, of the thrombus from travelling through the blood stream to other regions of the body, such as the lungs. The expandable device may be advanced along a guide wire, which may be a heated guide wire, to a position proximal of the thrombus and deployed (e.g., expanded) to impede fragments, such as emboli, of the thrombus from travelling through the blood stream to other regions of the body. In some variants, the expandable device may be heated, which may include having some portions heated and other portions not heated. For example, a distal portion of the expandable device, or a catheter delivering the expandable device, may be heated to facilitate passing through the thrombus. The expandable device, if heated, can be heated using at least the techniques described herein. The expandable device can be deployed by way of a catheter positioned distal of the thrombus and/or other occlusion, which can be after emulsification the heated guide wire is removed.

In some variants, the thermal thrombectomy systems disclosed herein may include a cutting device (e.g., thermal device), which may at least include a coil, lasso, corkscrew, drill, wire, spatula, auger, tapered wire drill, blade, knife, constricting coil, virectomy probe, ultrasonic cutter, extendable scrubbers (e.g., wires), umbrella, inverted mesh, and/or others. The cutting device, which may also be referred to as a scraping device and/or slicing device, may be advanced along the guide wire, which may be a heated guide wire, to the thrombus. In some variants, the cutting device may be deployed by way of a catheter positioned distal of the thrombus and/or other occlusion, which can be after the heated wire is removed. The cutting device may be manipulated to break up the thrombus. For example, the cutting device may be rotated, moved distally, moved proximally, vibrate, and/or otherwise manipulated. The cutting device may include mechanical, electrical, thermal, light-based, ultrasound, and/or other features. In some variants, the wire, which may be a heated guide wire, can include any of the foregoing features (e.g., coil, corkscrew, drill, wire, spatula, auger, tapered wire drill, etc.). The cutting device may be heated, which may include having a heated portion and an unheated portion, to facilitate breaking up the thrombus. In some variants, the cutting device can be used without an expandable device and/or separate guide wire. In some variants, the cutting device may have an oscillator and/or reciprocator to further ease the cutting or separation of the desired tissue.

In some variants, the thermal thrombectomy systems disclosed herein may include an aspiration device, which may at least include a wall vacuum, venturi vacuum, auger, syringe, oscillating vacuum with return filter, and/or other features. In some variants, the aspiration device may aspirate the thrombus out of the blood vessel. In some variants, the aspiration device may be heated, which may include having a heated portion and an unheated portion. For example, the aspiration device may include a mouth that can be heated. In some variants, a heated mouth of an aspiration device can be advanced distally to emulsify and aspirate a thrombus and/or other occlusion.

In some aspects, the techniques described herein relate to a thermal device configured to apply heat to a thrombus, the thermal device including: an outer tube including a distal portion; a heated element disposed at the distal portion of the outer tube; and one or more conduits configured to apply an electrical current to the heated element to raise a temperature of the heated element to apply heat to a thrombus.

In some aspects, the techniques described herein relate to a thermal device, further including an inner tube disposed within the outer tube that is configured to receive a guide wire, wherein the thermal device is configured to be advanced over the guide wire.

In some aspects, the techniques described herein relate to a thermal device, wherein the heated element includes a loop.

In some aspects, the techniques described herein relate to a thermal device, wherein the loop includes a nickel titanium alloy.

In some aspects, the techniques described herein relate to a thermal device, wherein the loop includes a tube.

In some aspects, the techniques described herein relate to a thermal device, wherein the loop includes a hollow lumen, and the thermal device further including a temperature sensor disposed within the hollow lumen.

In some aspects, the techniques described herein relate to a thermal device, further including a controller configured to modulate the electrical current applied to the heated element based on a sensed temperature by the temperature sensor.

In some aspects, the techniques described herein relate to a thermal device, further including a temperature sensor.

In some aspects, the techniques described herein relate to a thermal device, further including an electrode to sense leaked current.

In some aspects, the techniques described herein relate to a thermal device, wherein the electrode is a marker for visualization.

In some aspects, the techniques described herein relate to a thermal device, further including an expandable device configured to be expanded proximally of the thrombus.

In some aspects, the techniques described herein relate to a thermal device, wherein the expandable device includes a balloon.

In some aspects, the techniques described herein relate to a thermal device, wherein the expandable device includes a lumen through which the outer tube is configured to be advanced.

In some aspects, the techniques described herein relate to a thermal thrombectomy device configured to apply heat to a thrombus, the thermal thrombectomy device including: an expandable assembly including an expandable device, the expandable device configured to be expanded proximally of a thrombus; and a thermal assembly including a heated element, the thermal assembly configured to be advanced distally over a guide wire and out of the expandable assembly to apply heat to the thrombus with the heated element.

In some aspects, the techniques described herein relate to the thermal thrombectomy device, wherein the thermal assembly further includes one or more conduits configured to apply an electrical current to the heated element to raise a temperature of the heated element.

In some aspects, the techniques described herein relate to a thermal thrombectomy device, wherein the expandable device is a balloon.

In some aspects, the techniques described herein relate to a thermal thrombectomy device, wherein the heated element includes a loop.

In some aspects, the techniques described herein relate to a thermal thrombectomy device, wherein the loop includes a tube.

In some aspects, the techniques described herein relate to a thermal thrombectomy device, wherein the loop includes a hollow lumen, and the thermal thrombectomy device includes a temperature sensor disposed within the hollow lumen.

In some aspects, the techniques described herein relate to a thermal thrombectomy device, further including a controller configured to modulate the electrical current applied to the heated element based on a sensed temperature by the temperature sensor.

In some aspects, the techniques described herein relate to a thermal thrombectomy device, further including a temperature sensor.

In some aspects, the techniques described herein relate to a thermal thrombectomy device, further including an electrode to sense leaked current.

In some aspects, the techniques described herein relate to a method of crossing a thrombus, the method including: advancing a thermal assembly over a guide wire to a thrombus; and applying an electrical current to a heated element of the thermal assembly to heat the thrombus;

In some aspects, the techniques described herein relate to a method, further including advancing an expandable device over the guide wire to proximate the thrombus and expanding the expandable device.

In some aspects, the techniques described herein relate to a method, wherein the expandable device includes a balloon.

In some variants, a thermal thrombectomy device that can apply heat to a thrombus in a blood vessel of a patient is disclosed herein. The device can include a wire. The device can include an insulation layer disposed over at least a proximal portion of the wire to protect anatomy of a patient from heat. The device can include a temperature modulation unit that can apply energy to the wire to raise a temperature of the wire. The wire can be navigated through the vasculature of the patient to apply heat to a thrombus such that the thrombus softens or emulsifies (e.g., melts).

In some variants, the wire can include a nickel and titanium alloy.

In some variants, the insulation layer maybe a sheath.

In some variants, the device may include a power source. The power source can be a battery. In some variants, the wire is a guide wire.

In some variants, energy applied by the temperature modulation unit can be electricity.

In some variants, the temperature modulation unit can raise the temperature of the wire to heat collagen of the thrombus to 60 or more degrees Celsius. In some variants, the temperature modulation unit can raise the temperature of the wire to heat collagen of the thrombus to 60 or more degrees Celsius

In some variants, the temperature modulation unit can raise the temperature of the wire to heat collagen of the thrombus to 70 degrees Celsius. In some variants, the temperature modulation unit can raise the temperature of the wire to heat collagen of the thrombus to 80 degrees Celsius.

In some variants, the temperature modulation unit can raise the temperature of the wire to 60-80 degrees Celsius. In some variants, the temperature modulation unit can raise the temperature of the wire to 60 or more degrees Celsius.

In some variants, the temperature modulation unit can raise the temperature of the wire to 70 degrees Celsius. In some variants, the temperature modulation unit can raise the temperature of the wire to 80 or more degrees Celsius.

In some variants, the wire can include a coil.

In some variants, a thermal thrombectomy system that can apply heat to a thrombus in a blood vessel of a patient is disclosed herein. The system can include a wire. The system can include a layer of material surrounding a proximal portion of the wire. The system can include a power source that can apply energy to the wire to raise a temperature of the wire. The wire can be navigated through the vasculature of a patient to apply heat to a thrombus.

In some variants, the wire can be a guide wire.

In some variants, the layer of material can be a sheath.

In some variants, the layer of material can insulate anatomy of the patient from heat of the wire.

In some variants, the wire can include a nickel and titanium alloy.

In some variants, the power source can be a battery. In some variants, the electrical insulation comprises Polytetrafluoroethylene.

In some variants, the energy applied by the power source can be electricity.

In some variants, the power source can raise the temperature of the wire to heat the thrombus to 60-200 degrees Celsius. In some variants, the power source can raise the temperature of the wire to heat the thrombus to 60-200 degrees Celsius.

In some variants, the power source can raise the temperature of the wire to heat the thrombus to 70 degrees Celsius. In some variants, the power source can raise the temperature of the wire to heat the thrombus to 80 degrees Celsius.

In some variants, the power source can raise the temperature of the wire to 60-75 degrees Celsius. In some variants, the power source can raise the temperature of the wire to 60-80 degrees Celsius. In some variants, the power source can raise the temperature of the wire to 60-120 degrees Celsius.

In some variants, the power source can raise the temperature of the wire to 70 degrees Celsius. In some variants, the power source can raise the temperature of the wire to 80 degrees Celsius

In some variants, the wire can include a coil that can be disposed within the layer of material and deployed when proximate the thrombus.

In some variants, an outer periphery of the coil can be insulated.

In some variants, the wire can include two coils that can be overlaid on each other.

In some variants, the wire can include a coil drill.

In some variants, the wire can include a lasso. In some variants, the wire can include a loop.

In some variants, the wire can include a coil and a straight portion disposed through the coil.

In some variants, the wire can include an outer coil and an inner coil that can be joined together at a distal end. The inner coil can be heated and the outer coil may not be heated.

In some variants, the wire can include an outer coil and an inner coil that can be joined together at a distal end. The outer coil can be insulated.

In some variants, a method of performing a thrombectomy is disclosed herein. The method can include navigating a wire through vasculature of a patient to a thrombus. The method can include applying an energy source to the wire to raise a temperature of the wire. The method can include advancing the wire through the thrombus.

In some variants, the method can include advancing an expandable member along the wire to distal the thrombus.

In some variants, the method can include advancing a cutting device along the guide wire and into the thrombus.

In some variants, the method can include rotating the cutting device.

In some variants, the cutting device can include an auger.

In some variants, the method can include aspirating the thrombus.

In some variants, a method of performing a thrombectomy is disclosed herein. The method can include navigating a wire in a sheath through vasculature of a patient to a thrombus. The method can include applying an energy source to the wire to raise a temperature of the wire. The method can include advancing the sheath and wire into the thrombus. The method can include unsheathing a distal portion of the wire to allow the wire to self-expand. The method can include retracting the wire.

In some variants, wherein the wire self-expands to a coil and the method can include rotating the coil.

In some variants, the method can include aspirating the thrombus.

In some variants, a method of performing a thrombectomy is disclosed herein. The method can include navigating a wire in a sheath through vasculature of a patient to a thrombus. The method can include applying an energy source to the wire to raise a temperature of the wire. The method can include unsheathing a distal portion of the wire to allow the wire to self-expand. The method can include advancing the wire into the thrombus. The method can include retracting the wire.

In some variants, wherein the wire self-expands to a coil and the method can include rotating the coil.

In some variants, the method can include aspirating the thrombus.

In some variants, a thermal thrombectomy device is disclosed herein. The device can include a catheter that can include a port and a mount. The device can include a wire extending through the port. The wire can include an internal portion inside the catheter and external portion forming a lasso disposed outside the catheter. The end of the external portion can be coupled to the mount. The device can include a power source that can apply an electrical current to electrical contacts disposed at the port and mount to raise a temperature of the wire to soften or emulsify (e.g., melt) a thrombus. The device can include a cable extending through the catheter. The internal portion of the wire can be coupled to the cable. The cable can be rotated to extend more of the wire outside the catheter to increase a diameter of the lasso.

In some variants, the techniques described herein relate to a thermal thrombectomy system configured to remove a thrombus from a blood vessel of a patient, the system including: a capture device including a bag and a loop at an opening into the bag that is configured to be heated to apply heat to a thrombus, wherein the capture device is configured to be retracted proximally to interface the loop with the thrombus to detach the thrombus from a wall of a blood vessel, and wherein the bag is configured to catch the detached thrombus.

In some variants, the techniques described herein relate to a system, further including a catheter, wherein the capture device is configured to be deployed distally relative to the thrombus through the catheter.

In some variants, the techniques described herein relate to a system, further including a heated wire, the heated wire configured to penetrate the thrombus to facilitate distally positioning the capture device relative to the thrombus.

In some variants, the techniques described herein relate to a system, wherein further including an expandable device with a balloon configured to be inflated proximally of the thrombus.

In some variants, the techniques described herein relate to a system, wherein the temperature is between 60-200 degrees Celsius.

In some variants, the techniques described herein relate to a system, wherein the temperature is about 80 or more degrees Celsius.

In some variants, the techniques described herein relate to a system, wherein the temperature is between 60-80 degrees Celsius.

In some variants, the techniques described herein relate to a system, wherein the temperature is between 60-70 degrees Celsius.

In some variants, the techniques described herein relate to a system, further including a sheath configured to be disposed over the capture device.

In some variants, the techniques described herein relate to a system, wherein the loop includes a diameter that is larger than a diameter of a blood vessel.

In some variants, the techniques described herein relate to a system, further including an aspiration device configured to aspirate the thrombus.

In some variants, the techniques described herein relate to a system, wherein the aspiration device includes a mouth that is configured to be heated.

In some variants, the techniques described herein relate to a system, further including a temperature sensor configured to sense a temperature in the blood vessel.

In some variants, the techniques described herein relate to a system, wherein the system is configured to control the temperature of the loop based on the sensed temperature in the blood vessel.

In some variants, the techniques described herein relate to a method of performing a thrombectomy, the method including: positioning a capture device distal of a thrombus in a blood vessel; expanding a bag of the capture device; heating a loop disposed at a proximal opening into the bag to a temperature; proximally retracting the capture device to interface the loop with the thrombus to detach the thrombus from a wall of the blood vessel; catching the thrombus in the bag; and proximally retracting the capture device with the thrombus in the bag to remove the thrombus from the patient.

In some variants, the techniques described herein relate to a method, further including penetrating the thrombus with a heated wire.

In some variants, the techniques described herein relate to a method, wherein the temperature is between 60-200 degrees Celsius.

In some variants, the techniques described herein relate to a method, wherein the temperature is 80 or more degrees Celsius.

In some variants, the techniques described herein relate to a method, wherein the temperature is between 60-80 degrees Celsius.

In some variants, the techniques described herein relate to a method, wherein the temperature is between 60-70 degrees Celsius.

In some variants, the techniques described herein relate to a method, further including aspirating the thrombus with an aspiration device.

In some variants, the techniques described herein relate to a method, further including heating a mouth of the aspiration device.

In some variants, the techniques described herein relate to a method, further including sensing a temperature in the blood vessel and controlling the temperature of the loop based on the sensed temperature.

In some variants, the techniques described herein relate to a thermal thrombectomy system configured to remove a thrombus from a blood vessel of a patient, the system including: an aspiration device including a mouth that is configured to be heated to a temperature to soften or emulsify a thrombus, wherein the aspiration device is configured to be advanced distally to interface with and aspirate a thrombus.

In some variants, the techniques described herein relate to a system, wherein the temperature is between 60-200 degrees Celsius.

In some variants, the techniques described herein relate to a system, wherein the temperature is about 80 or more degrees Celsius.

In some variants, the techniques described herein relate to a system, wherein the temperature is between 60-80 degrees Celsius.

In some variants, the techniques described herein relate to a system, wherein the temperature is between 60-70 degrees Celsius.

In some variants, the techniques described herein relate to a thermal thrombectomy system configured to remove a thrombus from a blood vessel of a patient, the system including: a capture device including a bag; and a cutting device including a loop configured to be heated to a temperature to soften or emulsify a thrombus in a blood vessel; wherein the capture device and the cutting device are configured to be positioned distally of the thrombus and retracted proximally to interface the loop with the thrombus to detach the thrombus from the wall of the blood vessel and capture the detached thrombus in the bag.

In some variants, the techniques described herein relate to a system, further including a catheter, wherein the capture device and cutting device are configured to be deployed distally relative to the thrombus through the catheter.

In some variants, the techniques described herein relate to a system, further including a heated wire, the heated wire configured to penetrate the thrombus to facilitate distally positioning the capture device relative to the thrombus.

In some variants, the techniques described herein relate to a system, wherein a distal end of the capture device or a distal end of the cutting device includes a heated element configured to penetrate the thrombus to facilitate distally positioning the capture device and cutting device relative to the thrombus.

In some variants, the techniques described herein relate to a system, wherein the bag comprises a mesh.

In some variants, the techniques described herein relate to a system, wherein the temperature is 80 or more degrees Celsius.

In some variants, the techniques described herein relate to a system, wherein the temperature is between 60-80 degrees Celsius.

In some variants, the techniques described herein relate to a system, wherein the temperature is between 60-70 degrees Celsius.

In some variants, the techniques described herein relate to a system, further including a sheath configured to be disposed over the capture device.

In some variants, the techniques described herein relate to a system, further including a temperature sensor configured to sense a temperature in the blood vessel, wherein the system is configured to control the temperature of the loop based on the sensed temperature in the blood vessel.

In some variants, the techniques described herein relate to a system, wherein the capture device and cutting device are coupled together.

In some variants, the techniques described herein relate to a system, wherein an extension boom extends from a proximal opening into the bag to a distal end of the bag.

In some variants, the techniques described herein relate to a method of performing a thrombectomy, the method including: positioning a capture device and a cutting device distal of a thrombus in a blood vessel; expanding a bag of the capture device; heating a loop of the cutting device to a temperature; proximally retracting the cutting device to interface the loop with the thrombus to detach the thrombus from a wall of the blood vessel; catching the thrombus in the bag; and proximally retracting the cutting device and capture device with the thrombus in the bag to remove the thrombus from the patient.

In some variants, the techniques described herein relate to a method, further including penetrating the thrombus with a heated wire.

In some variants, the techniques described herein relate to a method, wherein the temperature is between 60-200 degrees Celsius.

In some variants, the techniques described herein relate to a method, wherein the temperature is about 80 or more degrees Celsius.

In some variants, the techniques described herein relate to a method, wherein the temperature is between 60-80 degrees Celsius.

In some variants, the techniques described herein relate to a method, wherein the temperature is between 60-70 degrees Celsius.

In some variants, the techniques described herein relate to a method, further including aspirating the thrombus with an aspiration device.

In some variants, the techniques described herein relate to a method, further including heating a mouth of the aspiration device.

In some variants, the techniques described herein relate to a method, further including sensing a temperature in the blood vessel and controlling the temperature of the loop based on the sensed temperature.

In some variants, the techniques described herein relate to a thrombectomy system including: a recessed surface that is configured to be deployed to engage a thrombus; and a conduit configured to provide energy to the recessed surface to heat the recessed surface to a temperature to soften or emulsify the thrombus.

In some variants, the techniques described herein relate to a thermal thrombectomy system, the system including: a member; and a temperature modulation unit configured to apply energy to the member to heat the member to a temperature; wherein the member is configured to be navigated through a blood vessel to apply heat to a thrombus.

In some variants, the techniques described herein relate to a system, wherein the member includes a wire.

In some variants, the techniques described herein relate to a system, wherein the energy is electrical current.

In some variants, the techniques described herein relate to a system, wherein the temperature is 60-200 degrees Celsius.

In some variants, the techniques described herein relate to a thermal thrombectomy system, the system including: an expandable device including one or more heated elements; and a temperature modulation unit configured to apply energy to the one or more heated elements to heat the one or more elements to a temperature; wherein the expandable device is configured to be retracted proximally to apply heat to a thrombus by way of the one or more heated elements.

In some variants, the techniques described herein relate to a system, wherein the expandable device includes an expandable bag configured to capture the thrombus.

In some variants, the techniques described herein relate to a system, wherein the energy is electrical current.

In some variants, the techniques described herein relate to a system, wherein the temperature is 60 or more degrees Celsius.

In some variants, the techniques described herein relate to a method of performing a thrombectomy, the method including: expanding an expandable device distal of a thrombus; applying energy to one or more heated elements of the expandable device to heat the one or more elements to a temperature; retracting the expandable device to interface the one or more heated elements with the thrombus; and capturing the thrombus in the expandable device.

In some variants, the techniques described herein relate to a method, wherein the expandable device includes an expandable bag configured to capture the thrombus.

In some variants, the techniques described herein relate to a method, wherein the energy is electrical current.

In some variants, the techniques described herein relate to a method, wherein the temperature is 60 or more degrees Celsius.

Neither the preceding summary nor the following detailed description purports to limit or define the scope of protection. The scope of protection is defined by the claims. Furthermore, reference is made herein to removing thrombi from veins. One of ordinary skill in the art will understand, after reviewing the entirety of this disclosure, that the systems and methods described herein may be applied to removing thrombi from arteries. Additionally, the systems and methods described herein can be used to remove other occlusions from the body.

### BRIEF DESCRIPTION OF THE DRAWINGS

The abovementioned and other features of the embodiments disclosed herein are described below with reference to the drawings of the embodiments. The illustrated embodiments are intended to illustrate, but not to limit, the scope of protection. Various features of the different disclosed embodiments can be combined to form further embodiments, which are part of this disclosure.

FIG. 1A illustrates a venous system in a limb.

FIG. 1B illustrates a vein with normal blood flow.

FIG. 1C illustrates a vein with a partial occlusion.

FIG. 1D illustrates a vein with a complete occlusion.

FIG. 2A illustrates a venous system in a limb.

FIG. 2B illustrates a vein with normal blood flow.

FIG. 2C illustrates a vein with early thrombus formation.

FIG. 2D illustrates a vein with a thrombus.

FIG. 2E illustrates an embolus breaking off of the thrombus.

FIG. 3A illustrates a venous system in a limb.

FIG. 3B illustrates a thrombus in a vein of the venous system causing swelling.

FIG. 4 illustrates a table showing the changes to a thrombus over time.

FIG. 5A illustrates an acute thrombus.

FIG. 5B illustrates a chronic thrombus.

FIG. 6 illustrates a thrombus disposed in a vein.

FIGS. 7A and 7B illustrate graphs showing the glass transition temperature of collagen in a thrombus and/or other thrombus forming elements.

FIG. 8 illustrates a thermal thrombectomy system.

FIGS. 9A-9D illustrate a method of using the thermal thrombectomy system.

FIGS. 10A-10E illustrate a method of using the thermal thrombectomy system.

FIG. 11 illustrates an expandable device that may impede distal movement of a thrombus or fragments thereof.

FIG. 12 illustrates an expandable device that may impede distal movement of a thrombus or fragments thereof.

FIGS. 13A-13C illustrates a method of using the thermal thrombectomy system with a wire having a coil.

FIGS. 14A-14C illustrates a method of using the thermal thrombectomy system with a wire having a tapered coil.

FIG. 15 illustrates a method of using the thermal thrombectomy system with a wire having a coil.

FIGS. 16A and 16B illustrate a cutting device with a spiral having a flat wire.

FIG. 17 illustrates a cutting device with variable scrubbers.

FIG. 18 illustrates a cutting device with a spiral and aspiration openings.

FIGS. 19A and 19B illustrate aspiration devices.

FIG. 20 illustrates a cross-sectional view of an aspiration device.

FIG. 21A illustrates a thermal thrombectomy system.

FIG. 21B illustrates a double coil for a wire of the thermal thrombectomy system.

FIG. 21C illustrates a double coil for a wire of the thermal thrombectomy system.

FIGS. 22A and 22B illustrate a method of using the thermal thrombectomy system.

FIG. 23 illustrates a sheath and a wire with a coil for the thermal thrombectomy system.

FIG. 24 illustrates a sheath and a wire with a lasso for the thermal thrombectomy system.

FIG. 25 illustrates a wire with a coil for the thermal thrombectomy system.

FIG. 26 illustrates a wire with a coil for the thermal thrombectomy system.

FIGS. 27A and 27B illustrate a wire with a double coil for the thermal thrombectomy system.

FIG. 28 illustrates a cutting device with flutes and a wire for the thermal thrombectomy system.

FIGS. 29A-29E illustrate a thermal thrombectomy system with variable lasso.

FIGS. 30A-30F illustrate cross-sectional views of veins.

FIG. 30G illustrates a cross-sectional view of a chronic thrombus in a vein.

FIGS. 31A-31C illustrate a heated element (e.g., heated wire) crossing over a thrombus.

FIGS. 31D-31F illustrate the deployment of a capture device (e.g., expandable device).

FIGS. 31G-31I illustrate the retraction of the capture device to capture the thrombus.

FIGS. 31J-31L illustrate the removal of the thrombus with the capture device.

FIG. 31M illustrates the vein with the thrombus removed.

FIG. 31N illustrates the removal of the thrombus away from the collection site through the vein.

FIG. 31O illustrates the removal of the thrombus and capture device from a patient through an aperture.

FIGS. 32A and 32B illustrate the positioning of the capture device distal of a thrombus.

FIGS. 32C-32E illustrate the deployment of a capture device with the retraction of a sheath.

FIGS. 32F-32H illustrate the retraction of the capture device to capture the thrombus.

FIGS. 33A-33C illustrate the retraction of a capture device with an outer diameter larger than the vessel diameter.

FIGS. 34A and 34B illustrate the retraction of the thrombus captured in the capture device to a mouth of a catheter.

FIGS. 34C-34H illustrate the retraction of the thrombus captured in the capture device into the heated mouth of the catheter.

FIGS. 35A-35F illustrate the aspiration of a thrombus through the advancing heated mouth of the catheter.

FIG. 36 illustrates a system for emulsifying and aspirating a thrombus.

FIG. 37 illustrates a device interface of the system.

FIG. 38 illustrates a thermal controller system.

FIG. 39A illustrates a system with a capture device (e.g., expandable device) and heated loop (e.g., thermal device, cutting device) deployable from a catheter.

FIG. 39B illustrates a cross-section of the system illustrated in FIG. 39A.

FIG. 39C illustrates a cross-section of the thermal device.

FIG. 39D illustrates a cross-section of the thermal device with example dimensions.

FIG. 39E illustrates a portion of the thermal device without an outer casing.

FIG. 39F illustrates a portion of the thermal device within the outer casing.

FIGS. 40A-40C illustrate a thermal device (e.g., cutting device).

FIG. 41A illustrates a thermal thrombectomy system.

FIG. 41B illustrates a cross-sectional view of the thermal thrombectomy system of FIG. 41A.

FIG. 42A illustrates a thermal thrombectomy system.

FIG. 42B illustrates a cross-sectional view of the thermal thrombectomy system of FIG. 42A.

FIG. 43A illustrates a thermal thrombectomy system.

FIG. 43B illustrates a cross-sectional view of the thermal thrombectomy system of FIG. 43A.

FIG. 44A illustrates a thermal thrombectomy system.

FIG. 44B illustrates a cross-sectional view of the thermal thrombectomy system of FIG. 44A.

FIG. 45 illustrates a capture device (e.g., expandable device).

FIGS. 46A-46F illustrate the emulsification of a thrombus during testing. FIG. 46A illustrates the thrombus disposed in a tube. FIG. 46B illustrates the thrombus after about one minute of exposure to a heated element. FIG. 46C illustrates the thrombus after about two minutes of exposure to the heated element. FIG. 46D illustrates the thrombus after about three minutes of exposure to the heated element. FIG. 46E illustrates the thrombus after about four minutes of exposure to the heated element. FIG. 46F illustrates the thrombus after about five minutes of exposure to the heated element.

FIGS. 47-51 illustrate distal ends that can crossover and/or core a thrombus and/or other occlusion.

FIG. 52 illustrates a thermal thrombectomy system.

FIG. 53A illustrates a handpiece for the thermal thrombectomy system.

FIG. 53B illustrates a thermal assembly of the thermal thrombectomy system.

FIG. 53C illustrates a connector of the thermal assembly.

FIG. 53D illustrates a distal end of the thermal assembly with a guide wire extending therethrough.

FIG. 53E illustrates the distal end of the thermal assembly with the guide wire retracted.

FIG. 53F illustrates internal features of the thermal assembly.

FIG. 54A illustrates an anchoring assembly of the thermal thrombectomy system.

FIG. 54B illustrates a connector of the anchoring assembly.

FIG. 54C illustrates an expandable device (e.g., balloon) of the anchoring assembly.

FIG. 54D illustrates a cross-section of the expandable device (e.g., balloon) of the anchoring assembly.

FIG. 55A illustrates a heated element of the thermal assembly being deployed through an inside of a sheath of the anchoring assembly.

FIG. 55B illustrates the heated element of the thermal assembly deployed through the inside of the sheath of the anchoring assembly.

FIG. 55C illustrates a tube of the thermal assembly disposed through the connector of the anchoring assembly.

### DETAILED DESCRIPTION

Although certain embodiments and examples are described below, this disclosure extends beyond the specifically disclosed embodiments and/or uses and obvious modifications and equivalents thereof. Thus, it is intended that the scope of this disclosure should not be limited by any particular embodiments described below. Furthermore, this disclosure describes many embodiments in reference to veins and arteries; the systems and methods described in relation to veins can be applied to arteries and those described in relation to arteries can be applied to veins.

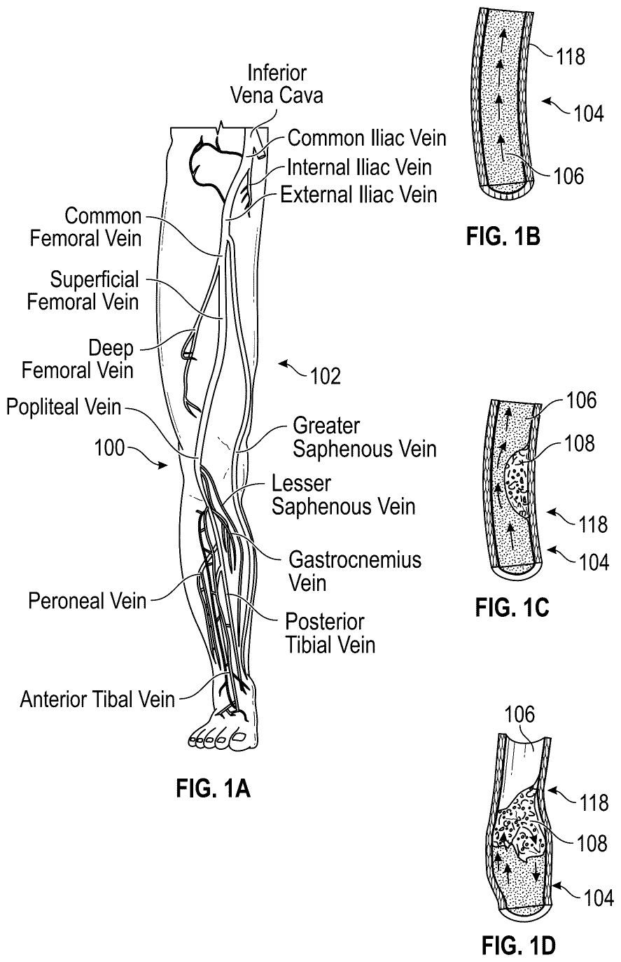

FIG. 1A illustrates a limb 100 (e.g., leg, arm) of a person. The limb 100 includes a venous system 102 that carries deoxygenated blood from the limb 100 back to the heart.

FIG. 1B illustrates a cross-section of a portion of a vein 104 of the venous system 102 with normal blood flow. The vein 104 includes a flow path 106 for the deoxygenated blood bounded by the wall 118 of the vein 104. A thrombus 108 may develop within a vein 104, as illustrated in FIG. 1C, to partially restrict (e.g., impede, occlude, etc.) blood flow through the 106. The thrombus 108 may form on the wall 118 of the vein 104, which may include a valve. The wall 118 may from along the wall 118 for a variety of reasons, which may include damage to the wall 118, inactivity, diet, and/or other reasons. The thrombus 108 may grow in size to completely block (e.g., impede, occlude, etc.) blood flow through the flow path 106, as shown in FIG. 1D. The occlusion of the blood flow can result in swelling, pain, and/or discoloration of the limb 100. The removal of thrombi can relieve a patient of the foregoing symptoms as well as prevent or at least slow further symptoms from developing.

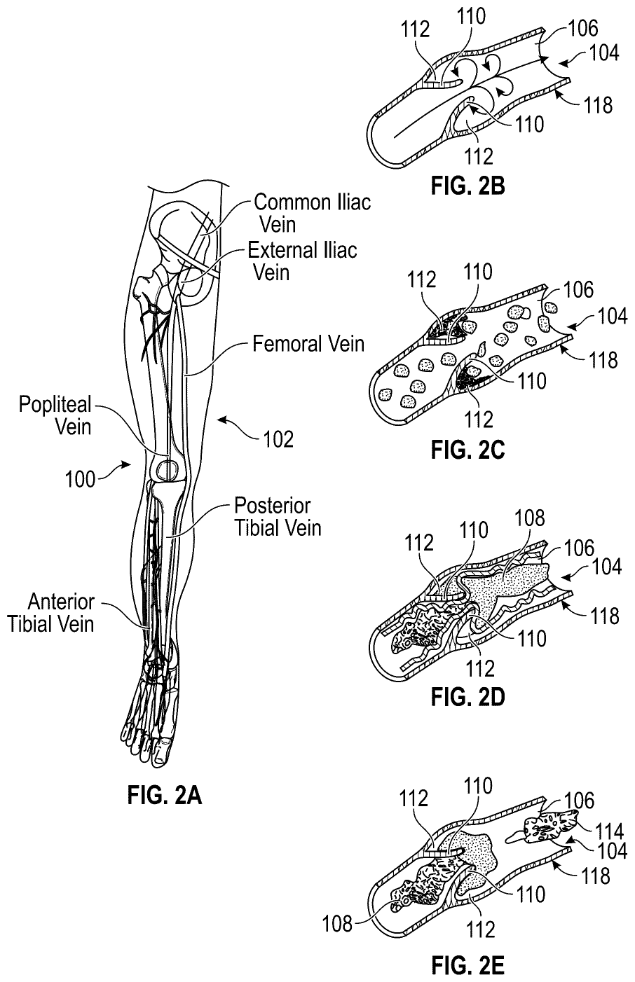

FIG. 2A illustrates a limb 100 (e.g., leg, arm) of a person. The limb 100 includes venous system 102 that carries deoxygenated blood from the limb 100 back to the heart.

FIG. 2B illustrates a cross-section of a portion of a vein 104 of the venous system 102 with normal blood flow. The vein 104 includes a flow path 106 for the deoxygenated blood bounded by the wall 118 of the vein 104. The vein 104 may include valves 110. The valves 110 may open to allow blood to flow toward the heart and close to prevent backflow. Pockets 112 can be disposed downstream of the valves 110 between the valves 110 and the wall 118 of the vein 104. As shown, a portion of blood in the flow path 106 may flow into the pockets 112. A thrombus may begin to develop on the wall 118 of the vein 104, valves 110, and/or in the pockets 112, as shown in FIG. 2C, until a thrombus 108 is formed, as illustrated in FIG. 2D. The thrombus 108 may fill the flow path 106, which can include extending around the valves 110 and into the pockets 112. The thrombus 108 may prevent proper function of the valves 110. The thrombus 108 may partially or completely block blood flow through the vein 104. In some instances, an embolus 114 may break off from the thrombus 108, as illustrated in FIG. 2E. The embolus 114 may travel to other regions of the body, which can include traveling to the lungs and result in a pulmonary embolism (PE)—a potentially lethal condition. Accordingly, the removal of thrombi may help a patient avoid a lethal condition.

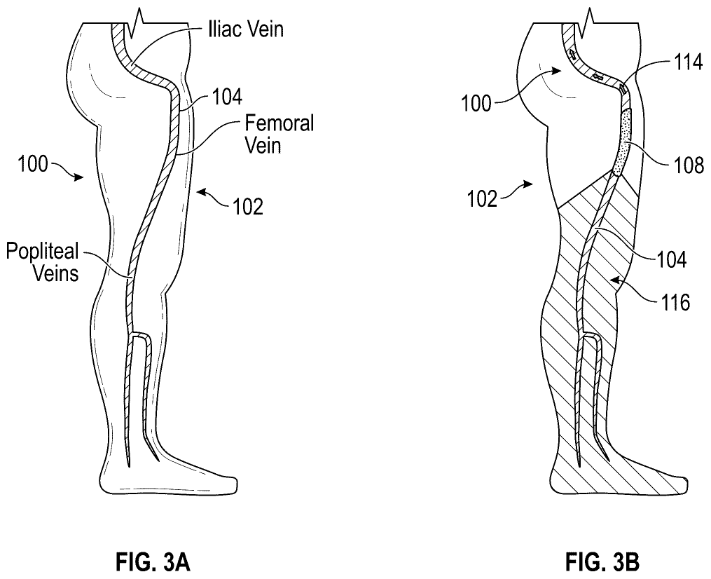

FIG. 3A illustrates a limb 100 (e.g., leg, arm) of a person. The limb 100 includes a venous system 102 with multiple veins 104. As shown in FIG. 3B, a thrombus 108 may form in the vein 104 to block blood flow. The blocked blood flow may prevent drainage of blood from the limb 100 and cause swelling of the limb 100 below the thrombus 108 as indicated by portion 116. As detailed herein, an embolus 114 may separate from the thrombus 108 and travel to other regions of the body, which can include traveling to the lungs and result in a pulmonary embolism (PE).

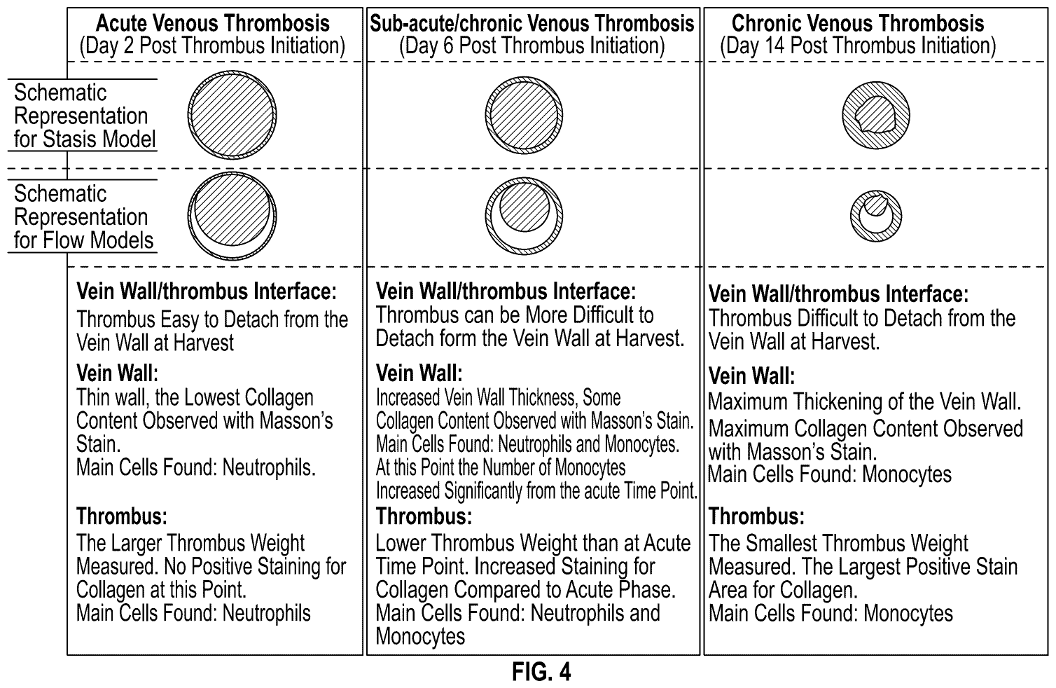

As a thrombus ages, the characteristics of the proximate portion of the vein and the thrombus itself may change, as indicated in the table shown in FIG. 4.

For example, initially after formation (e.g., two days post thrombus initiation), a thrombus may be referred to as an acute thrombus. The vein wall proximate the acute thrombus may be thin and have a low collagen content. The main cells found in the vein wall proximate the acute thrombus may be neutrophils. The acute thrombus itself may have no or relatively little collagen. The main cells found in the acute thrombus may be neutrophils. The acute thrombus may be readily detached from the vein wall and/or penetrated.

After some additional time (e.g., six days post thrombus initiation), a thrombus may be referred to as a sub-acute/chronic thrombus. The vein wall proximate the sub-acute/chronic thrombus may be thickened and have a higher collagen content compared to the acute thrombus period. The main cells found in the vein wall proximate the sub-acute/chronic thrombus may be neutrophils and monocytes with the quantity of monocytes significantly increased compared to the acute thrombus period. The sub-acute/chronic thrombus itself may have an increased collagen content compared to the acute thrombus. The main cells found in the sub-acute/chronic thrombus may be neutrophils and monocytes. The sub-acute/chronic thrombus may be more difficult to detach from the vein wall compared to the acute thrombus and/or more difficult to penetrate. The sub-acute/chronic thrombus may have a lower weight compared to the acute thrombus.

After some additional time (e.g., fourteen days post thrombus initiation), a thrombus may be referred to as a chronic thrombus. The vein wall proximate the chronic thrombus may be thickened and have a higher collagen content compared to the sub-acute/chronic thrombus period. The main cells found in the vein wall proximate the chronic thrombus may be monocytes. The chronic thrombus may have an increased collagen content compared to the sub-acute/chronic thrombus. The main cells found in the chronic thrombus may be monocytes. The chronic thrombus may be more difficult to detach from the vein wall compared to the sub-acute/chronic thrombus and/or more difficult to penetrate. The chronic thrombus may have a lower weight compared to the sub-acute/chronic thrombus.

The hardness of a thrombus may increase over time, which may be due to the increase in collagen content. For example, a chronic thrombus may be harder than a sub-acute/chronic thrombus which may be harder than an acute thrombus. A harder thrombus may be more difficult to penetrate, which can make removal more difficult. Additionally, as indicated above, a chronic thrombus may be harder to detach from the vein wall compared to the sub-acute/chronic thrombus which may be harder to detach from the vein wall compared to the acute thrombus.

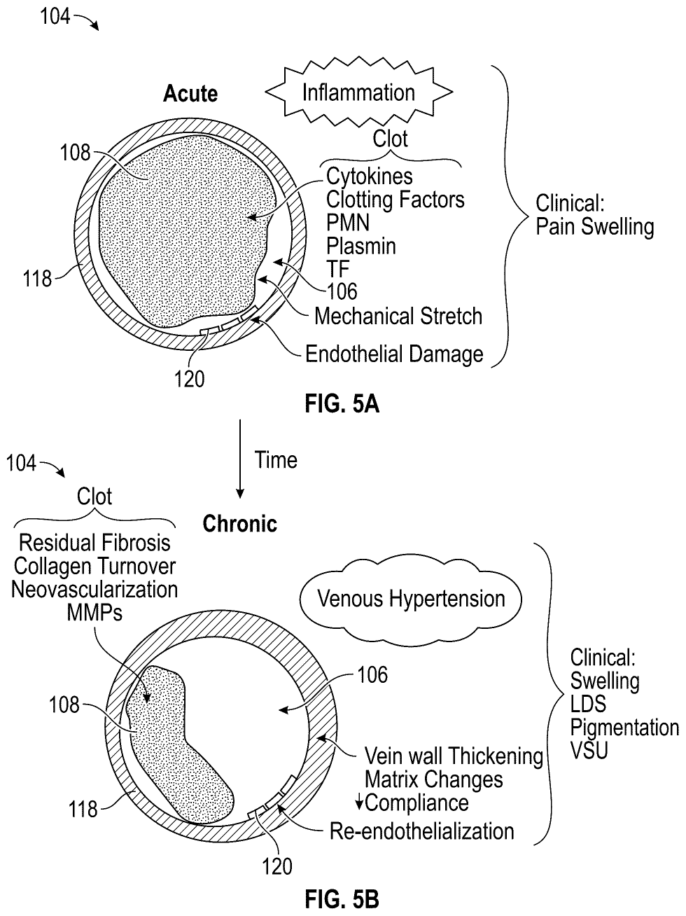

As shown in FIG. 5A, an acute thrombus may be accompanied by inflammation of the proximate vein wall as well as stretching of the proximate vein wall. The acute thrombus may also damage the endothelial cells along the vein wall. The patient may experience pain and swelling in the region of the body with the acute thrombus. The acute thrombus may progress into a chronic thrombus over time. The chronic thrombus may be accompanied by venous hypertension, thickening of the vein wall, and matrix changes within the vein wall. The patient may at least experience swelling and pigmentation in the region of the body with the chronic thrombus.

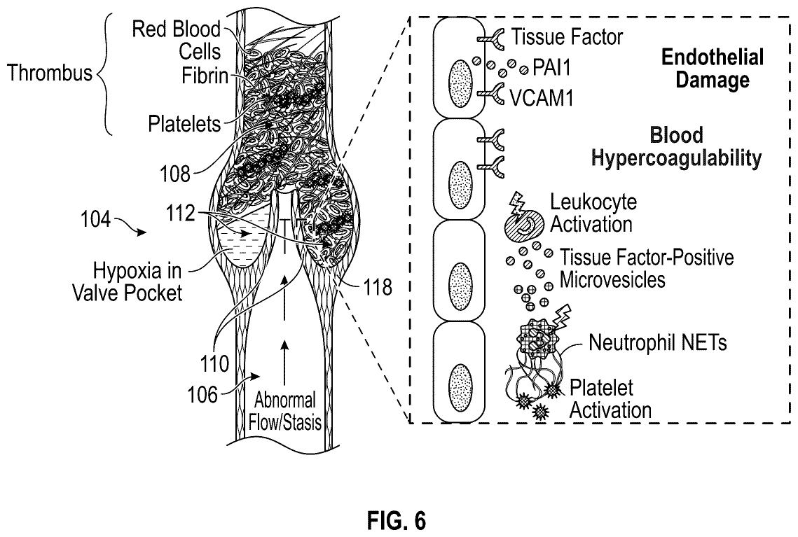

FIG. 6 illustrates another example section of a vein 104 with a thrombus 108. The thrombus 108 is disposed against the wall 118 and valve 110 of the vein 104. The thrombus 108 is disposed in the pockets 112. As illustrated, the thrombus 108 may result in hypoxia in the pockets 112 and surrounding tissue. During a thrombectomy procedure, the thrombus 108 is penetrated, but as explained, the thrombus 108 may become harder overtime making penetration more difficult. Additionally, the thrombus 108 may be increasingly harder to detach from the wall 118 and/or valves 110 as time passes. Accordingly, at least due to the foregoing reasons, a surgeon may need to perform multiple passes with a thrombectomy device to remove the thrombus. With each pass, the wall 118 may be scrapped in an effort to remove the thrombus. The likelihood of damaging the wall 118 and/or valves 110 of the vein 104 increases with the number of passes. The thermal thrombectomy systems and methods disclosed herein may reduce the number of passes, which may include reducing to a single pass, to remove a thrombus, decreasing the likelihood of damage to the wall 118 and/or valves 110 which may decrease the risk of re-thrombosis. The thermal thrombectomy systems and methods disclosed herein may reduce the overall ease and/or time of a procedure, which may lower the risk of complications and improve efficiency.

As described herein, the thermal thrombectomy systems and methods disclosed herein may include heating a thrombus. Heating the thrombus may ease penetration of the thrombus. Heating the thrombus may ease breaking the thrombus apart. Heating the thrombus may ease detachment of the thrombus from the wall of the blood vessel. Heating the thrombus may soften and/or emulsify (e.g., melt, liquefy) the thrombus (e.g., One or more proteins (e.g., collagen) and/or other thrombus forming elements (e.g., biopolymers). The thrombus may soften and/or emulsify when heated to a temperature within 60-300 degrees Celsius, 60-200 degrees Celsius, 60-120 degrees Celsius (e.g., 80 degrees Celsius), 60-70 degrees Celsius, and/or 60-80 degrees Celsius. Accordingly, the heated elements (e.g., wires, loops, lassos, hoops, mouths, and/or others) described herein may be heated to 60-300 degrees Celsius, 60-200 degrees Celsius, 60-120 degrees Celsius (e.g., 80 degrees Celsius), 60-70 degrees Celsius, 60-80 degrees Celsius, or other temperatures. For example, the heating element can be heated to 64-70 degrees Celsius, 66-70 degrees Celsius, 68-70 degrees Celsius, 70-72 degree Celsius, 72-74 degree Celsius, 74-76 degree Celsius 76-78 degree Celsius and/or 78-80 degrees Celsius. The heating element can be heated to 64-66 degrees Celsius and/or 66-68 degrees Celsius. The heating element can be heated to about 80 degrees Celsius. The heating element can be heated to any temperature within the foregoing ranges. The temperature of the heating element can be modulated and/or controlled based on temperatures at the heating element and/or interface between the heating element and the thrombus and/or other occlusion. For example, the systems and methods disclosed herein can use one or more temperature sensors (e.g., thermocouples) to sense temperatures at the heating element and/or interface between the heating element and the thrombus and/or other occlusion, and based on the sensed temperatures, modulate and/or control the temperature of the heating element. The system and/or method described herein can include insulation to protect anatomy of the patient from heat.

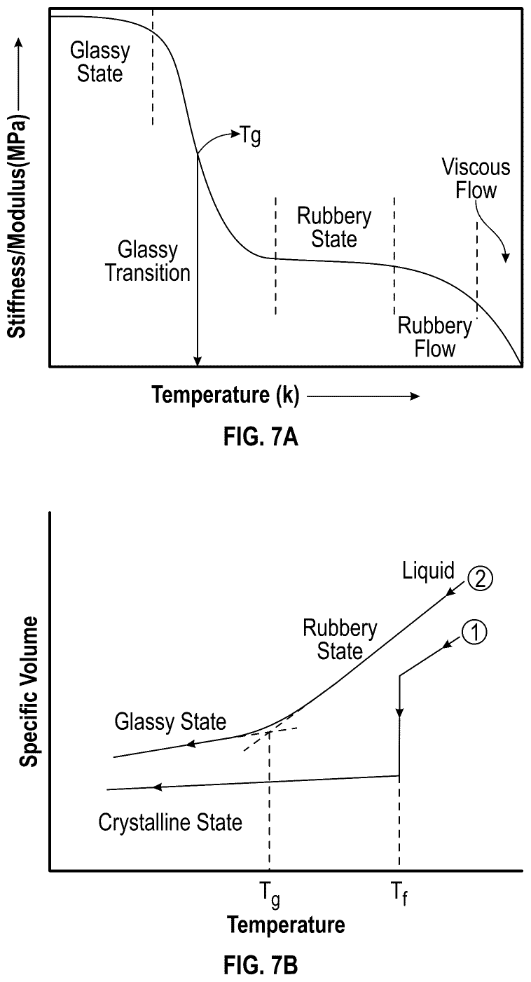

Heating the thrombus may facilitate a glass transition of the thrombus (e.g., soften), such as the glass transition of the one or more proteins (e.g., collagen) and/or other thrombus forming elements (e.g., biopolymers). For example, FIG. 7A illustrates an example graph representing the stiffness/modulus (MPa) against temperature (k) of a thrombus, which can include one or more proteins (e.g., collagen) and/or other thrombus forming elements. As shown, at a glass transition temperature (Tg), the thrombus, which can include one or more of the forming elements, may reach a glass transition where they may start flowing more readily in the glassy state (solid to liquid). FIG. 7B illustrates an example graph representing the specific volume of the thrombus, which can include one or more proteins (e.g., collagen) and/or other thrombus forming elements, against temperature. Once again, at a glass transition temperature (Tg), the thrombus, which can include one or more proteins (e.g., collagen) and/or other thrombus forming elements, may reach a glass transition. When heated to the glass transition temperature, the thrombus, which can include one or more proteins (e.g., collagen) and/or other thrombus forming elements, may change from a first state (e.g., hard state) to a second state softer than the first state. When heated to the glass transition temperature, the thrombus which can include one or more proteins (e.g., collagen) and/or other thrombus forming elements, may decrease in viscosity.

The thermal thrombectomy systems and methods described herein may heat the thrombus, which can include one or more proteins (e.g., collagen) of the thrombus and/or other thrombus forming elements, to a temperature sufficient to soften and/or emulsify (e.g., melt, liquefy) the thrombus, which can include the one or more proteins (e.g., collagen) and/or other thrombus forming elements, and/or facilitate a glass transition of the thrombus, which can include one or more proteins (e.g., collagen) and/or other thrombus forming elements. The thermal thrombectomy systems and methods described herein may heat the thrombus, which can include collagen of the thrombus and/or other thrombus forming elements, to decrease the viscosity of the thrombus, which can include collagen of the thrombus and/or other thrombus forming elements. The temperature to soften the thrombus, which may include one or more proteins and/or other thrombus forming elements, may be the glass transition temperature. The temperature to emulsify (e.g., melt, liquefy) the thrombus, which may include one or more proteins and/or other thrombus forming elements, may be higher than the transition temperature (e.g., melting temperature).

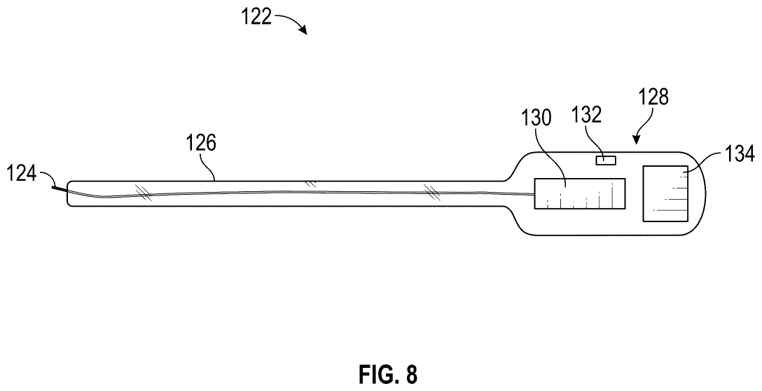

FIG. 8 illustrates an example thermal thrombectomy system 122, which may be a device (e.g., reusable device, disposable device, partially disposable device, etc.). The thermal thrombectomy system 122 may include a wire 124, which may be a guide wire. The wire 124 may be at least solid or braided. The wire 124 may include one or more materials. For example, the wire 124 may include a metal, such as steel (e.g., stainless steel) and/or an alloy of nickel and titanium (e.g., Nitinol). The wire 124 may include a coating, which may include a TEFLON® and/or parylene coating. The wire 124 may be straight. The wire 124 may include one or more expandable features, such as a coil, corkscrew, drill, auger, tapered wire drill, constricting coil, scrubbers, umbrella, mesh, bag, and/or others.

The thermal thrombectomy system 122 may include various software and hardware components to implement aspects of this disclosure, which may at least include a temperature modulation unit 130, controller 132, and/or power source 134 or at least an interface to receive energy from a power source. The wire 124 may be operatively connected to a temperature modulation unit 130. The temperature modulation unit 130 may adjust the temperature of the wire 124 (e.g., heating element, heater, etc.). For example, the temperature modulation unit 130 may raise the temperature (e.g., heat) the wire. The temperature modulation unit 130 may heat the wire 124 directly or indirectly with one or more energy sources, which may at least include heat, radio frequency, laser, electricity (e.g., current), resistive heating, inductive heating, nuclear, heated liquid, and/or others. For example, the temperature modulation unit 130 may apply a current of electricity to the wire 124, raising the temperature of the wire 124. In some variants, the temperature modulation unit 130 may lower the temperature (e.g., cool) the wire. The temperature of the wire 124 may be heated to the temperatures described herein. The temperature of the wire 124 may be automatically modulated based on monitored conditions (e.g., sensed with a temperature sensor) in a blood vessel and/or at thrombus. The temperature of the wire 124 may be adjusted to accommodate for convection losses. The temperature of the wire 124 may be controlled by a surgeon. The controller 132 may be operatively connected with the temperature modulation unit 130 to perform the temperature control described herein by way of the temperature modulation unit 130.

The power source 134 may be a battery, which may include a rechargeable battery and/or disposable one-time-use battery. The power source 134 may power the thermal thrombectomy system 122, which can include the controller 132 and/or temperature modulation unit 130. The power source 134 may supply the energy to adjust the temperature (e.g., heat) the wire 124. In some variants, the thermal thrombectomy system 122 may be operatively connected to an external power source.

The thermal thrombectomy system 122 may also include memory, communication interface(s) (wired or wireless), user interfaces (e.g., button(s), dial(s), switch(es), display(s), touchpad(s), touchscreen(s), knob(s), trigger(s), indicator(s), gauge(s), and/or slider(s)), etc. to implement aspects of the disclosure. The temperature modulation unit 130, controller 132, power source 134, and/or other components of the thermal thrombectomy system 122 may be housed in a housing 128, which may be a handle.

The thermal thrombectomy system 122 may include a sheath 126, which may also be referred to as a covering or insulation. The sheath 126 may insulate the wire 124 from the anatomy of the patient, such as the blood vessel. The sheath 126 may be disposed over a proximal portion of the wire 124, which may include being disposed over the entirety of the wire 124 except a distal portion. The wire 124, in some variants, may be deployed from and retracted into the sheath 126. In some variants, the sheath 126 may be an insulating coating disposed on the wire 124.

In use, the wire 124 may be navigated through the vascular system to a thrombus. In some variants, the wire 124 can be routed through a catheter to the thrombus. The wire 124 may be heated by the temperature modulation unit 130 to soften and/or emulsify the thrombus, which can include one or more proteins (e.g., collagen) and/or other thrombus forming elements. The wire 124 may be heated by the temperature modulation unit 130 and then advanced into the thrombus to ease penetration, which may be especially advantageous when penetrating a chronic thrombus. As the wire 124 advances through the thrombus, the heat from the wire 124 may soften and/or emulsify the thrombus, which can include the one or more proteins (e.g., collagen) and/or other thrombus forming elements. In some variants, the wire 124 may pass through the thrombus prior to heating, which may enable an expandable device to be disposed distal of the thrombus prior to heating.

In some variants, the wire 124 may include one or more cutting and/or scraping features (e.g., coil, corkscrew, drill, auger, tapered wire drill, constricting coil, scrubbers, umbrella, mesh, and/or others). The one or more cutting features may scape the wall of the blood vessel proximate the thrombus to detach the thrombus from the wall of the blood vessel. The one or more cutting features may cut the thrombus to break up the thrombus. The one or more cutting features may engage with the thrombus to capture the thrombus, such that the surgeon may extract the thrombus by way of retracting the wire 124 proximally out of the patient. The one or more cutting features may be heated to soften and/or emulsify the thrombus, which can include one or more proteins (e.g., collagen) and/or other thrombus forming elements. In some variants, a cutting device and/or an expandable collection device, separate from the wire 124, may be advanced along the wire 124, which may be by way of a sheath or catheter. The cutting device and/or expandable collection device may be heated or not. The sheath 126 may protect the blood vessel from the heat of the wire 124.

The thermal thrombectomy system 122 may include an expandable collection device (e.g., bag, inverted mesh, mesh, cage, umbrella, balloon, basket, funnel, etc.) that may be disposed distally of the thrombus to avoid the thrombus or a fragment (e.g., embolus) thereof breaking off and traveling to another region of the body. In some variants, the wire 124 may integrally include the expandable device. In some variants, an expandable device, separate from the wire 124, may be advanced over the wire 124 with a sheath and/or catheter. The expandable device, in some variants, may include heated portions.

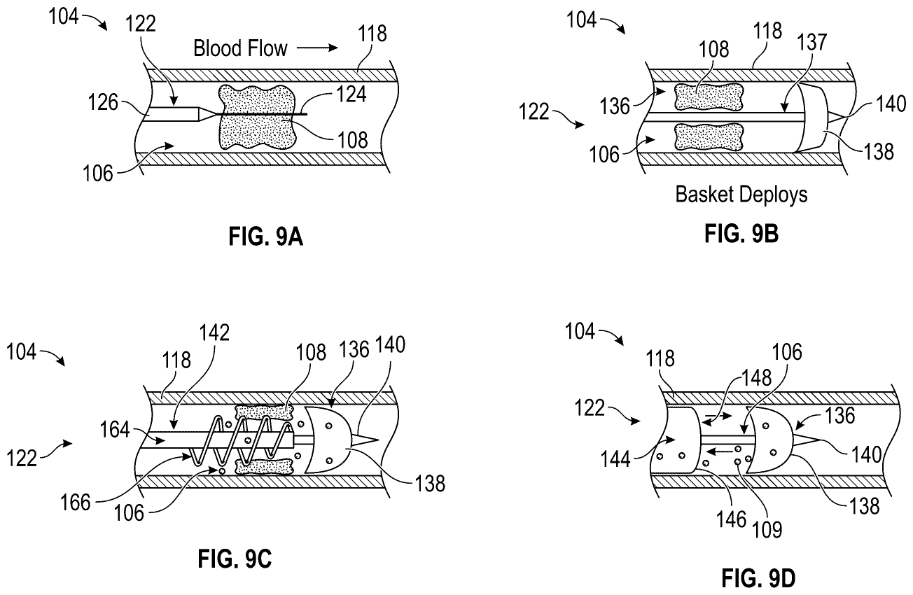

FIGS. 9A-9D illustrate an example method of using the thermal thrombectomy system 122 to remove a thrombus. As shown in FIG. 9A, the distal end of the wire 124 (e.g., guide wire) may be navigated through the vascular system to a thrombus in a blood vessel, such as a vein 104. The wire 124 may be heated by the temperature modulation unit 130. The wire 124 may be heated to account for the convection losses from the blood flow through the flow path 106. In some variants, the wire 124 may include a sensor to detect heat. The wire 124 may be heated to a temperature sufficient to raise the temperature of the thrombus, which can include the one or more proteins (e.g., collagen) of the thrombus and/or other thrombus forming elements, to soften and/or emulsify.

The distal portion of the heated wire 124 may be advanced through the thrombus 108, softening and/or emulsifying the thrombus, which can include the one or more proteins (e.g., collagen) and/or other thrombus forming elements, such that the distal end of the wire 124 is positioned distal of the thrombus 108. The wire 124 may continue to apply heat to the thrombus once through or may cease applying heat. The sheath 126 may insulate the portions of the blood vessel not proximate the thrombus from the heated wire 124. In some variants, the wire 124 may be passed through the thrombus 108 before applying heat via the wire 124.

As illustrated in FIG. 9B, an expandable device 136 (e.g., expandable collection device) may be advanced along the wire 124 and through the thrombus 108 until an expandable end 138, such as a bag (e.g., mesh, braded, etc.), inverted mesh, umbrella, balloon, basket, funnel (e.g., mesh funnel), disc, and/or other device, of the expandable device 136 is distal of the thrombus 108. The expandable end 138 may be deployed (e.g., expanded) distal of the thrombus 108 to prevent the thrombus 108 and/or a portion thereof from traveling distally to another region of the body. The expandable end 138 and/or a distal portion of the expandable device 136 may include a tip 140. In some variants, the tip 140 may be heated to help the expandable end 138 pass through the thrombus 108. In some variants, a periphery of the expandable end 138 extending to the wall 118 of the vein 104 may be heated, which may help detach the thrombus 108 from the wall 118. The expandable device 136 can be advanced along the wire 124 with a catheter 137 or sheath. In some variants, portions of the catheter 137 or sheath may be heated, which may include a distal portion.

As illustrated in FIG. 9C, a cutting device 142 may be advanced along the wire 124 to the thrombus 108. The cutting device 142 may be advanced along the wire 124 and/or the catheter 137 with a catheter 164 to position a feature, which can include an auger 166 or other feature (e.g., coil, drill, corkscrew, tapered wire drill, constricting coil, etc.) to cut, penetrate, and/or scrape the thrombus 108, proximate the thrombus 108. In some variants, the auger 166 or other feature may be advanced while rotating to cut into, thread into, and/or break up the thrombus 108. The auger 166 may scrape the wall 118 of the vein 104 to detach the thrombus 108. The auger 166 may be radially inset from the wall 118 of the vein 104 to detach the thrombus 108. The auger 166 and/or other portions of the cutting device 142, which may include the catheter 164, can be heated. The cutting device 142 may be retracted through a catheter 144.

As illustrated in FIG. 9D, the catheter 144 may be an aspiration device, which may include a channel for instrumentation, an intake channel 146 for fragments 109 of the thrombus 108, and a return channel 148 for returning filtered blood removed from the flow path 106 by way of the intake 146. The catheter 144 may be advanced toward the expandable end 138 of the expandable device 136 to aspirate the fragments 109 of the thrombus 108 into the intake channel 146. In some variants, the expandable end 138 of the expandable device 136 may be retracted proximally toward the catheter 144 such that the intake channel 146 may aspirate the fragments 109 of the thrombus 108. In some variants, a portion of the catheter 144, such as the distal end, may be heated. The wire 124 or any other features discussed may be heated or not heated during any of the steps discussed in reference to FIGS. 9A-9D.

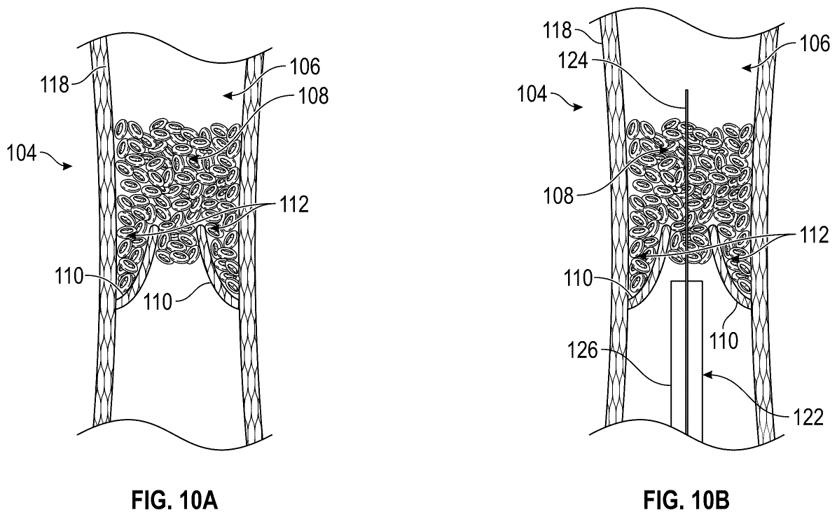

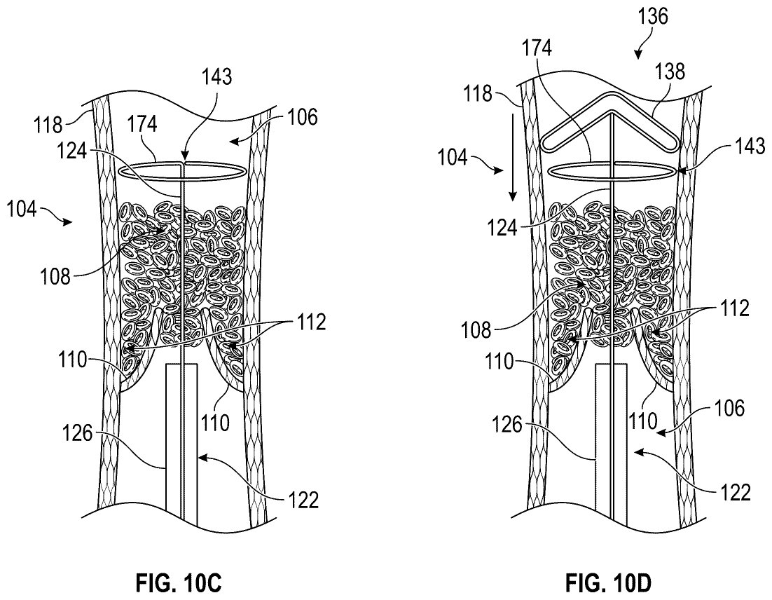

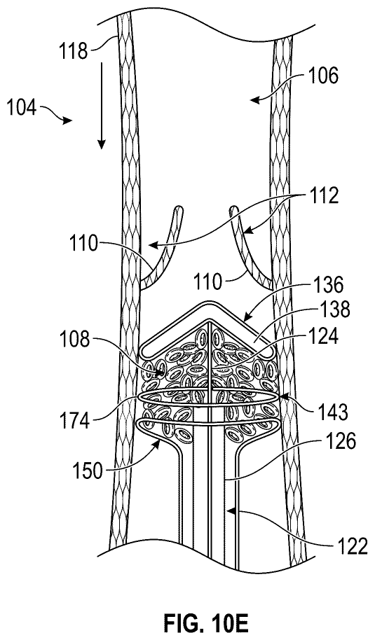

FIGS. 10A-10E illustrate an example method of using the thermal thrombectomy system 122 to remove a thrombus. As illustrated in FIG. 10A, a thrombus 108 may form along the wall 118 and/or valve 110 of a vein 104 to impede flow. As shown in FIG. 10B, a distal end of the wire 124 may be navigated to pass through the thrombus 108 to a distal side. The wire 124 may be heated as described herein, which may help the wire 124 penetrate the thrombus 108. The heated wire 124 may soften and/or emulsify the thrombus, which can include the one or more proteins (e.g., collagen) of the thrombus and/or other thrombus forming elements, which may make the thrombus easier to break and/or easier to detach from the wall 118. The sheath 126, which may be a catheter, can protect the vein 104 from damage.

As illustrated in FIG. 10C, a cutting device 143 may be deployed (e.g., expanded) from the distal portion of the wire 124, which may include a portion of the wire 124 distal of the thrombus 108. The cutting device 143 may include a lasso (e.g., loop) and/or any of the other cutting features described herein. In some variants, the cutting device 143 may be a separate component from the wire 124 and be advanced along the wire 124 (e.g., with a catheter or sheath) to a position distal of the thrombus 108.

As illustrated in FIG. 10D, an expandable end 138, which may include any of the expandable features described herein (e.g., umbrella, bag, balloon, etc.), of an expandable device 136 may be deployed (e.g., expanded) from a distal end of the wire 124, which may include a portion of the wire 124 distal of the thrombus 108. The expandable device 136 may impede the thrombus 108 and/or fragments thereof from travelling distally to other regions of the body. In some variants, the expandable device 136 may be a separate component from the wire 124 and may be advanced along the wire 124 (e.g., with a catheter or sheath) to a position distal of the thrombus 108 prior to expansion of the expandable end 138.

As illustrated in FIG. 10E, the wire 124, cutting device 143, and/or expandable device 136 may be retracted together proximally to remove the thrombus 108. The wire 124, cutting device 143, and/or expandable device 136 may be retracted through a funnel 150 (e.g., mouth) that may be positioned on a distal portion of a catheter (e.g., sheath 126) to guide the thrombus 108 into the catheter with the wire 124, cutting device 143, and/or expandable device 136 for removal. The funnel 150 may be deployed (e.g., expanded) once the catheter is disposed proximate the thrombus 108. The wire 124, cutting device 143, and/or expandable device 136 may be retracted proximally to remove the thrombus 108 from the patient. In some variants, the wire 124, cutting device 143, expandable device 136, funnel 150, and/or catheter may be retracted proximally together to remove the thrombus 108.

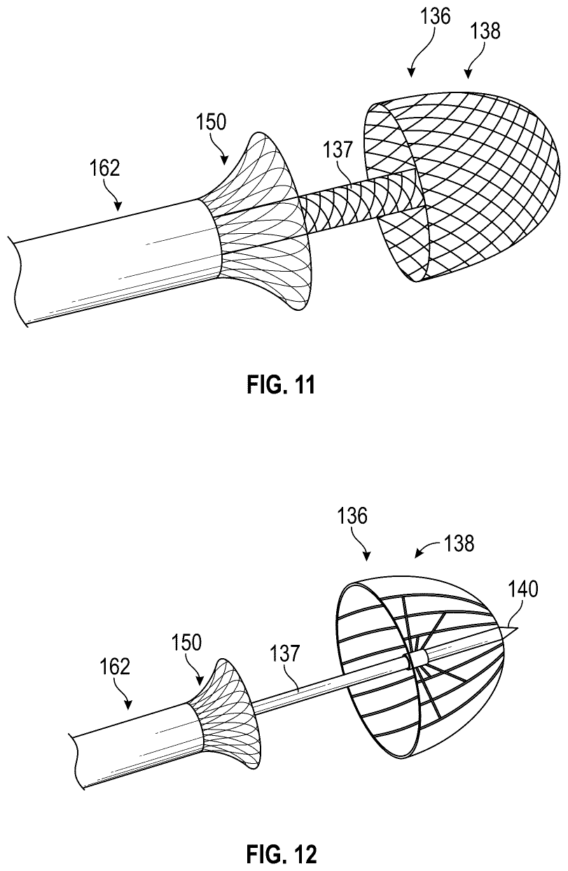

FIG. 11 illustrates an example expandable device 136 (e.g., collection device, capture device), which may be passive. The expandable device 136 may be advanced through a catheter 162, which may be an outer catheter. The expandable device 136 may include a catheter or sheath 137. The catheter 137 may be advanced along a guide wire and through a thrombus to position an expandable end 138 of the expandable device 136 distal of the thrombus. With the expandable end 138 distal of the thrombus, the expandable end 138 may be deployed (e.g., expanded). The expandable end 138 illustrated in FIG. 11 shows an inverted mesh but any of the expandable devices/ends described herein can be used. The expandable device 136 may prevent the thrombus and fragments thereof from travelling distally to other regions of the body during a procedure. The expandable device 136 may be retracted proximally to urge the thrombus or fragments thereof in a proximal direction, which may include into a catheter, funnel, or the like, for removal. A funnel 150, which may be mesh, may be disposed at a distal portion of the catheter 162 to prevent the thrombus and fragments thereof from moving proximally past the distal opening of the catheter 162 without entering. In some variants, the funnel 150 can be retracted with the expandable device 136, trapping the thrombus and fragments thereof between the expandable end 138 and the funnel 150 for removal. Any portion of the expandable end 138 may be heated using at least the techniques described herein.

FIG. 12 illustrates an example expandable device 136, which may be active. The expandable device 136 may be advanced through a catheter 162, which may be an outer catheter 162. The expandable device 136 may include a catheter or sheath 137. The catheter 137 may be advanced along a guide wire and through a thrombus to position an expandable end 138 of the expandable device 136 distal of the thrombus. With the expandable end 138 distal of the thrombus, the expandable end 138 may be deployed (e.g., expanded). The expandable end 138 illustrated in FIG. 12 shows an umbrella but any of the expandable devices/ends described herein can be used. The expandable device 136 may prevent the thrombus and fragments thereof from travelling distally to other regions of the body during a procedure. The expandable device 136 may be retracted proximally to urge the thrombus or fragments thereof in a proximal direction, which may include into a catheter, funnel, or the like, for removal. A funnel 150 may be disposed at a distal portion of the catheter 162 to prevent the thrombus and fragments thereof from moving proximally past the distal opening of the catheter 162 without entering. In some variants, the funnel 150 can be retracted with the expandable device 136, trapping the thrombus and fragments thereof between the expandable end 138 and the funnel 150 for removal. Any portion of the expandable end 138 may be heated using at least the techniques described herein.

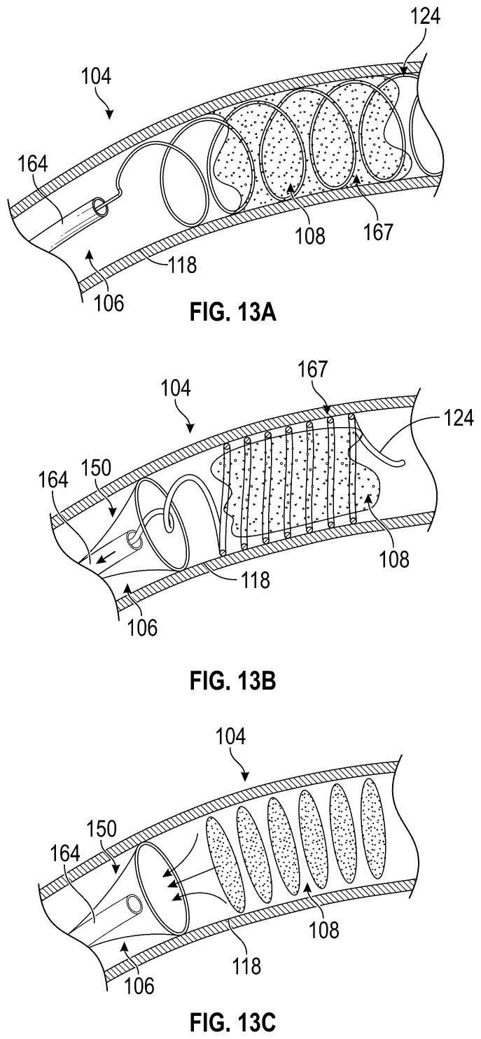

FIGS. 13A-13C illustrate an example method of using the thermal thrombectomy system 122 with the wire 124 having a coil 167 (e.g., helical coil). The distal end of the wire 124 may be navigated to the location of a thrombus 108. In some variants, the wire 124 may be navigated to the location of the thrombus 108 in a catheter 164, which may also be referred to as a sheath, and with the distal end of the catheter 164 proximate the thrombus 108, the distal end of the wire 124 may be advanced out of the catheter 164 to proximate the thrombus 108. The coil 167 may self-expand outside the catheter 164. As described herein, an energy source may be applied to the wire 124 to heat it. For example, an electrical current may be applied to the wire 124, heating the wire 124. The wire 124 may be heated to a temperature that will soften and/or emulsify the thrombus, which can include one or more proteins (e.g., collagen) and/or other thrombus forming elements, which may be any of the other temperatures described herein. The coil 167 or any other coil described herein may be disposed at the vessel wall or radially inward of the vessel wall.

As illustrated in FIG. 13A, the wire 124, which may be heated, can be advanced distally and rotated, such that the coil 167 spirals around and/or through (e.g., threads into) the thrombus 108. As the coil 167 is advanced and rotates, the coil 167 can scrape the wall 118 of the vein 104 to separate the thrombus 108 therefrom. The coil 167 may be radially inset from the wall 118 to avoid scraping the wall 118. The heat of the wire 124 may ease penetration of the wire 124 through the thrombus 108 and/or ease detachment from the wall 118 of the vein 104 as the thrombus, which can include the one or more proteins (e.g., collagen) and/or thrombus forming elements, are softened and/or emulsified. The heat from the wire 124 may ease breaking of the thrombus 108.

As illustrated in FIG. 13B, with the coil 167 around and/or through the thrombus 108, the wire 124 may be retracted, fragmenting the thrombus 108. The heating of the wire 124 may ease fragmentation of the thrombus 108. A funnel 150 may be disposed proximal of the thrombus 108 to direct the thrombus 108 and/or fragments thereof traveling proximally to be directed through the funnel 150 and into the catheter 164 and/or another catheter for removal from the patient. The wire 124, including the coil 167, maybe retracted through the catheter 164 for removal. As illustrated in FIG. 13C, the thrombus 108, which may include fragments thereof, may be aspirated from the blood vessel (e.g., vein or artery) through the catheter 164 and/or another catheter. An expandable device, including at least those described herein, may be positioned distal of the thrombus 108. In some variants, only an inside periphery of the coil 167 is heated and/or an outside of the periphery of the coil 167 is insulated.

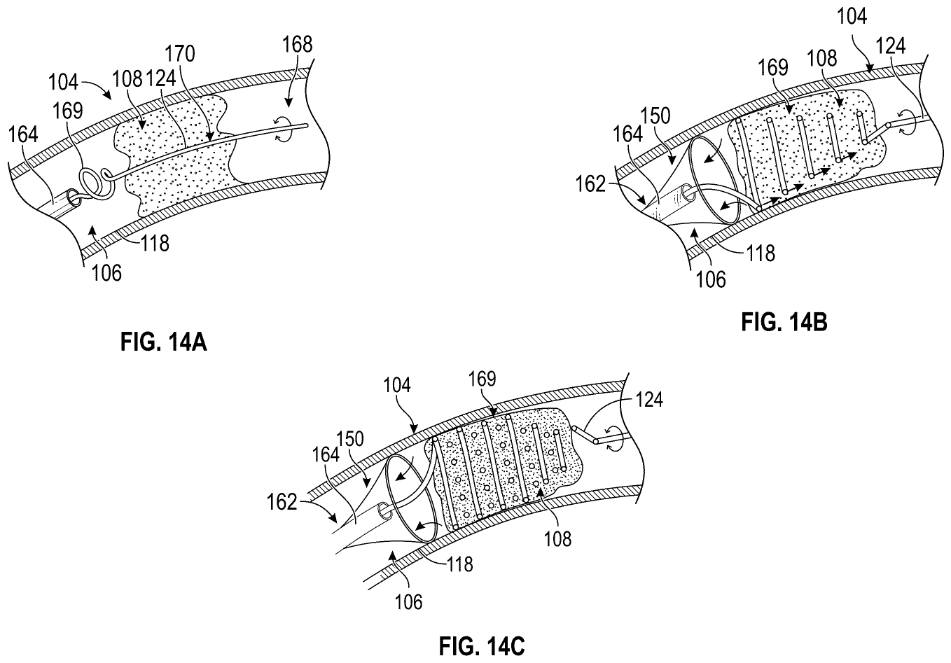

FIGS. 14A-14C illustrate an example method of using the thermal thrombectomy system 122 with the wire 124 having a tapered coil 169 (e.g., helical coil, wire drill, etc.). The tapered coil 169 may be tapered in a proximal to distal direction with the distal portion having a diameter that is smaller than the proximal portion. The tapered coil 169 may be positioned proximal relative to a straight distal portion 170 of the wire 124. In some variants, the tapered coil 169 may be tapered in a distal to proximal direction with the proximal portion having a diameter that is smaller than the distal portion. The tapered coil 169, in some variants, may be positioned at the distal tip of the wire 124.

The distal end of the wire 124 may be navigated to the location of a thrombus 108. In some variants, the wire 124 may be navigated to the location of the thrombus 108 in a catheter 164, which may also be referred to as a sheath, and with the distal end of the catheter 164 proximate the thrombus 108, the distal end of the wire 124 may be advanced out of the catheter 164 to proximate the thrombus 108. The tapered coil 169 may self-expand outside the catheter 164. As described herein, an energy source may be applied to the wire 124 to heat it. For example, an electrical current may be applied to the wire 124, heating the wire 124. The wire 124 may be heated to a temperature that will soften and/or emulsify the thrombus, which can include one or more proteins (e.g., collagen) and/or other thrombus forming elements, which may at least include any temperature described herein.

As illustrated in FIG. 14A, the wire 124, which may be heated, can be advanced distally such that the straight portion 170 penetrates the thrombus 108. The heat of the wire 124 may ease penetration into the thrombus 108. The wire 124 may continue to be advanced past the straight portion 170 and rotated such that the tapered coil 169 threads (e.g., spirals) into the thrombus 108 and/or detaches the thrombus 108 from the vessel walls, as illustrated in FIG. 14B. The pathway of the tapered coil 169 may cut the various planes, which may include all planes, of the thrombus 108. A funnel 150 may be positioned proximally of the thrombus 108 to facilitate aspiration through the catheter 164 and/or outer catheter 162. The funnel 150 and outer catheter 162 may be positioned over the catheter 164. The funnel 150 may be disposed on a distal end of the outer catheter 162. As illustrated in FIG. 14C, the pathways (e.g., tracks) of the coils of the tapered coil 169 may create holes (e.g., tunnels, channels, etc.) through the thrombus 108 to ease aspiration. The tapered coil 169 may fragment the thrombus 108. The tapered coil 169 when retracted proximally may fragment and/or detach the thrombus 108 from the walls of the vessel. The thrombus 108, or at least a portion thereof, may be captured by the tapered coil 169 such that retraction of the wire 124 retracts the thrombus 108 through the funnel 150 and into the catheter 162 and/or catheter 164, which can include aspiration or not. The fragmented thrombus 108 may be extracted by way of the catheter 164 and/or catheter 162 by retraction of the tapered coil 169 through the catheter 164 and/or aspiration through the catheter 164 and/or catheter 162. An expandable device, including at least those described herein, may be positioned distal of the thrombus 108. In some variants, only an inside periphery of the tapered coil 169 is heated and/or an outside of the periphery of the tapered coil 169 is insulated.

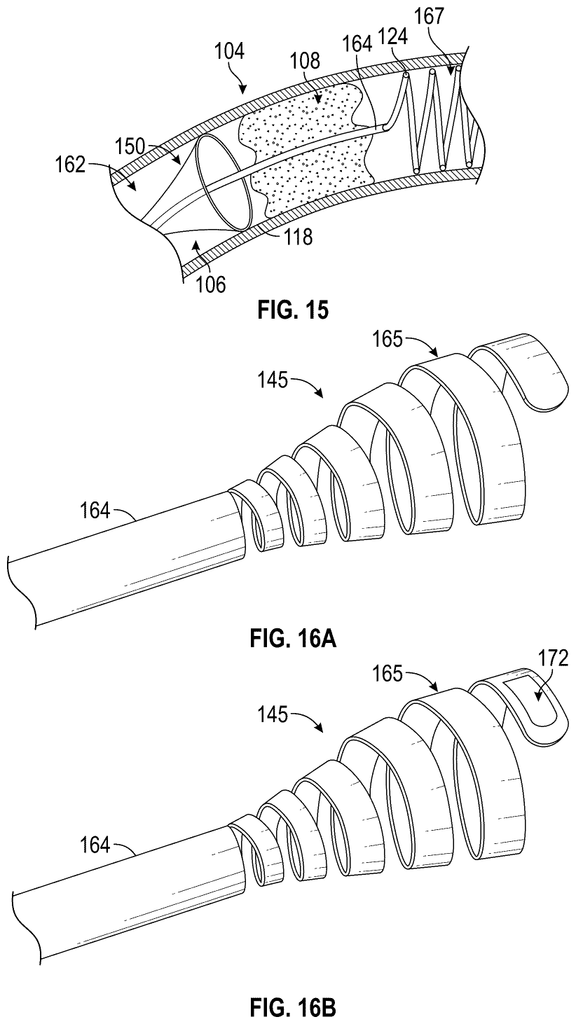

FIG. 15 illustrates the thermal thrombectomy system 122, wherein the catheter 164 carrying the wire 124 and coil 167 carries the coil 167 distal of the thrombus 108 before the coil 167 is deployed from the catheter 164. The catheter 164 can be navigated to the thrombus 108. The catheter 164 may be advanced to penetrate and pass through the thrombus 108. In some variants, a distal portion of the wire 124, which may be heated, may protrude from the distal end of the catheter 164 to ease penetration. In some variants, the distal portion of the catheter 164 may be heated to ease penetration through the thrombus 108. With the distal end of the catheter 164 distal of the thrombus 108, the wire 124 may be advanced such that the coil 167 is positioned distal of the catheter 164 and thrombus 108. The coil 167 may self-expand outside the catheter 164. The catheter 164 may be retracted proximally and back into the outer catheter 162. The coil 167 may be retracted proximally and/or rotated to cut into the thrombus 108 and/or detach the thrombus 108 from the walls of the vessel, which may fragment the thrombus 108. The thrombus 108 may be captured by the coil 167 such that, as the wire 124 is retracted back into the catheter 164 disposed in the catheter 162, the thrombus 108 is moved into the catheter 164 and/or catheter 162. The catheter 164 and/or catheter 162 may aspirate the thrombus 108. A expandable device, including at least those described herein, may be positioned distal of the thrombus 108.

FIGS. 16A and 16B illustrate a cutting device 145, which may also be referred to as a scraping device. The cutting device 145 may include a catheter 164, which may also be referred to as a sheath. The cutting device 145 may include a spiral 165, which may also be called a coil, tapered coil, spatula, spatula spiral, etc. The spiral 165 may be tapered in a distal to proximal direction with the proximal portion comprising a diameter that is smaller than the distal portion. The spiral 165 may extend from the distal portion of the catheter 164. The spiral 165 may include a flat member (e.g., flat wire) that forms the spiral shape. The width of the flat member in the proximal-distal direction may get larger as the spiral 165 extends farther away from the catheter 164. The flat member may have a cross section that is generally rectangular, which may include having rounded ends. The flat member may have a cross section wherein the proximal-distal length is the longest dimension. The cutting device 145 may be advanced to proximal a thrombus. The cutting device 145 may be advanced over a guide wire to proximal a thrombus. The cutting device 145 may be advanced distally and/or rotated such that the spiral 165 cuts into the thrombus and/or detaches the thrombus from the vessel wall. The spiral 165 may be heated to ease detachment of the thrombus from the vessel wall and/or cutting into the thrombus. For example, the spiral 165 may include a heated portion 172 that may be heated to soften and/or emulsify the thrombus, which can include one or more proteins (e.g., collagen) and/or other thrombus forming elements. The shape of the flat member of the spiral 165 may avoid damage to the vessel wall and/or valves as the spiral 165 detaches the thrombus from the vessel walls. In some variants, the spiral 165 may fragment the thrombus and/or capture the thrombus (e.g., portion thereof).

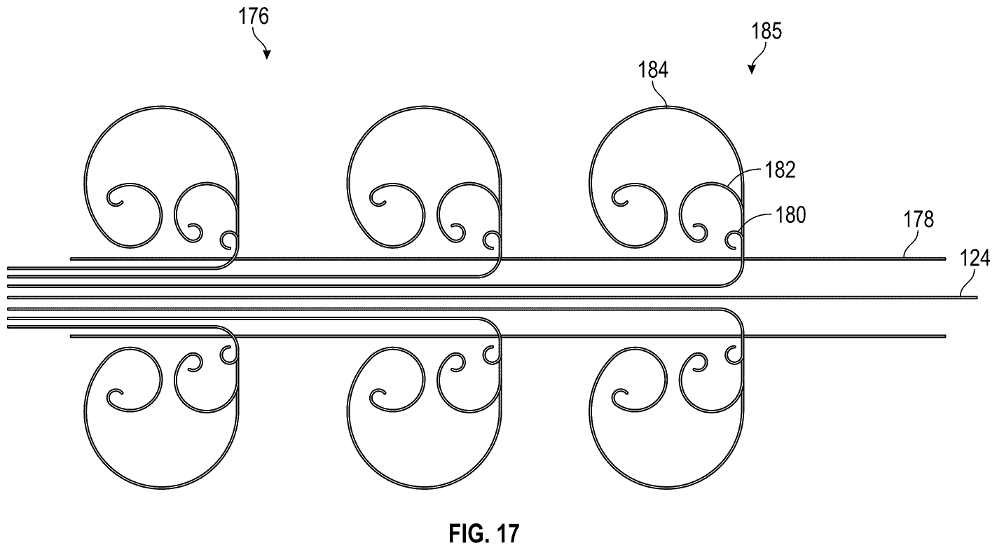

FIG. 17 illustrates a cutting device 176, which may also be referenced to as a scrubber device and/or variable scrubber device. The cutting device 176 may include a catheter 178, which may also be referred to as a sheath. The cutting device 176 may include a plurality of scrubbing members 185, which may also be referred to as cutting members, scraping members, etc. The scrubbing members 185 may be distributed along a longitudinal length of the cutting device 176, which may include a portion (e.g., distal portion) of the longitudinal length. The scrubbing members 185 may be circumferentially distributed about the circumference of the catheter 178. The scrubbing members 185 may extend radially outward to different radial distances. For example the scrubbing members 185 may include a first scrubber 180 that may extend radially to a first radial distance, second scrubber 182 that can extend radially to a second radial distance greater than the first radial distance, and/or a third scrubber 184 that extends radially to a third radial distance greater than the second radial distance. In some variants, the scrubbing members 185 may extend from an interior of the catheter 178 to an exterior. In some variants, the scrubbing members 185 may not extend from the interior of the catheter 178 to the exterior. In some variants, the scrubbing members 185 may be fixedly attached to and extend from an outer surface of the catheter 178. In some variants, the scrubbing members 185 may be wires. The wires may curve (e.g., loop) in a proximal direction. The radius of curvature of the third scrubber 184 may be larger than a radius of curvature of the second scrubber 182 which may be larger than a radius of curvature of the first scrubber 180. In some variants, the cutting device 176 may be advanced along a wire 124 for positioning. Portions of the cutting device 176 may be heated, which may at least include the scrubbing members 185, third scrubber 184, second scrubber 182, first scrubber 180, and/or catheter 178. The cutting device 176 may be advanced and/or retracted to pass through a thrombus. The scrubbing members 185 may cut into the thrombus and/or detach the thrombus from the vessel walls. The scrubbing members 185 may fragment the thrombus.

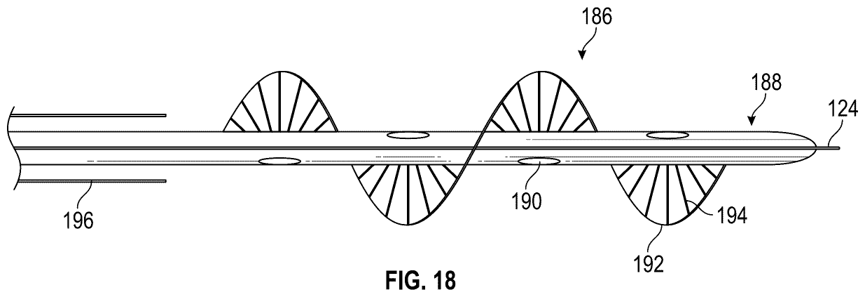

FIG. 18 illustrates a cutting device 186. The cutting device 186 may include cutting features and/or aspirations features. The cutting device 186 may include a catheter 188, which may be a sheath, to facilitate advancement along a wire 124. The cutting device 186 may be deployed distally from inside a catheter 196, which may be stationary. The cutting device 186 may aspirate thrombi. The cutting device 186 may include an expandable feature such as a spiral 192 (e.g., coil). The spiral 192 may extend radially outward. The spiral 192 may self-expand when the coil 192 is positioned outside the catheter 196. The spiral 192 can include a plurality of struts 194 that support the spiral 192 in the expanded configuration. The strut 194 may extend from an exterior of the catheter 188. The spiral 192 may spiral around an exterior of the catheter 188. The spiral 192 can spiral along a distal portion of the catheter 188. The cutting device 186 may include aspiration openings 190. The aspiration openings 190 may be disposed through the catheter 188 to aspirate thrombi. The aspiration openings 190 may distributed along a distal portion of the catheter 188. The aspiration openings 190 may be distributed around the spiral 192. Portions of the cutting device 186 may be heated, which may at least include the catheter 188, spiral 192, strut 194, peripheries of the aspiration openings 190, and/or other portions. The cutting device 186 may be advanced distally (or retracted proximally) and rotated to cut a thrombus with the spiral 192 and/or detach the thrombus from the vessel wall. The aspiration openings 190 may aspirate fragments of the thrombus. The catheter 196 may aspirate fragments of the thrombus. The spiral 192 may capture a thrombus or at least portions thereof such that, upon retraction, the thrombus may be removed by way of the catheter 196.

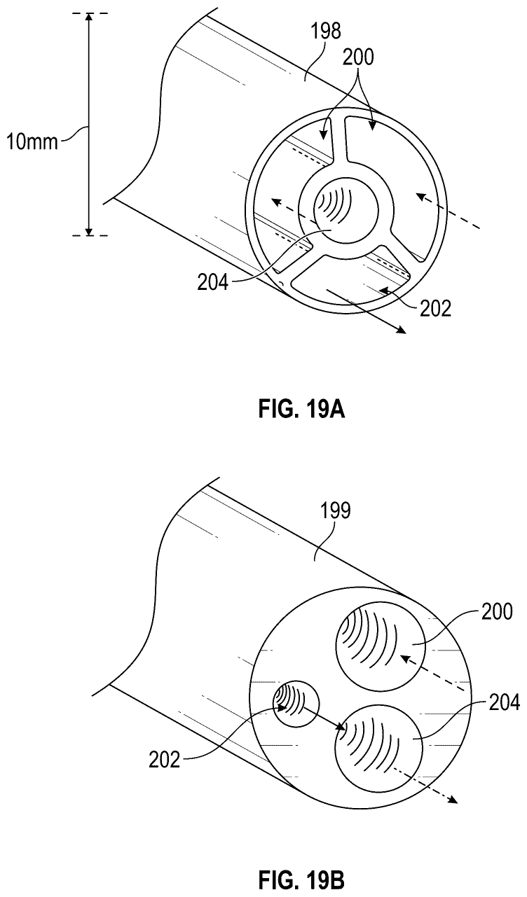

FIG. 19A illustrates an aspiration device 198, which may also be referred to as a catheter and/or aspiration catheter, that can aspirate a thrombus from a blood vessel. The aspiration device 198 may include a plurality of channels. The aspiration device 198 may include an instrumentation channel 204. Guide wires, expandable devices, cutting devices, catheters, and/or other instruments may be routed (e.g., advanced, retracted, etc.) through the instrumentation channel 204. The aspiration device 198 may include intake channel(s) 200 (e.g., one, two, three, or more intake channels) to aspirate fragments of a thrombus. The aspiration device 198 may include return channel(s) 202 (e.g., one, two, three, or more return channels) to return filtered blood received through the intake channel(s) 200 to the blood vessel. The total cross-sectional flow area of the intake channel(s) 200 may be greater than that of the return channel 202. Ribs can separate the various channels of the aspiration device 198. The instrumentation channel 204 may be centered in the aspiration device 198. The intake channel(s) 200 and/or return channel(s) 202 may be distributed around the instrumentation channel 204.

FIG. 19B illustrates an aspiration device 199, which may also be referred to as a catheter and/or aspiration catheter, that can aspirate a thrombus from a blood vessel. The aspiration device 199 may include an instrumentation channel 204, intake channel(s) 200, and/or return channel(s) 202. Each of the instrumentation channel 204, intake channel(s) 200, and/or return channel 202 may be off-center of the center of the aspiration device 199. The instrumentation channel 204, intake channel(s) 200, and/or return channel(s) 202 may have circular peripheries. The intake channel(s) 200 and instrumentation channel 204 may be the same size. The intake channel 200 may be larger in cross-sectional size than the return channel 202.

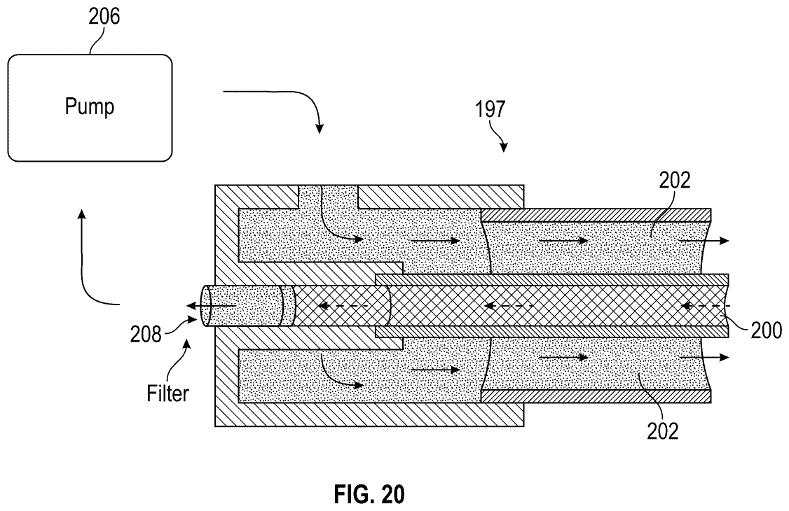

FIG. 20 illustrates a cross-section of an aspiration device 197 that can aspirate a thrombus from a blood vessel. The aspiration device 197 may include a return channel 202 and an intake channel 200. The intake channel(s) 200 may be centered along a longitudinal axis of the aspiration device 197. The aspiration device 197 may be operatively connected to a pump 206 that aspirates fragments of a thrombus and blood through the intake channel 200. The intake channel(s) 200 may direct the thrombus and blood through a filter 208, filtering the thrombus from the blood. The pump 206 may return the filtered blood back into the blood vessel by way of the return channel 202.

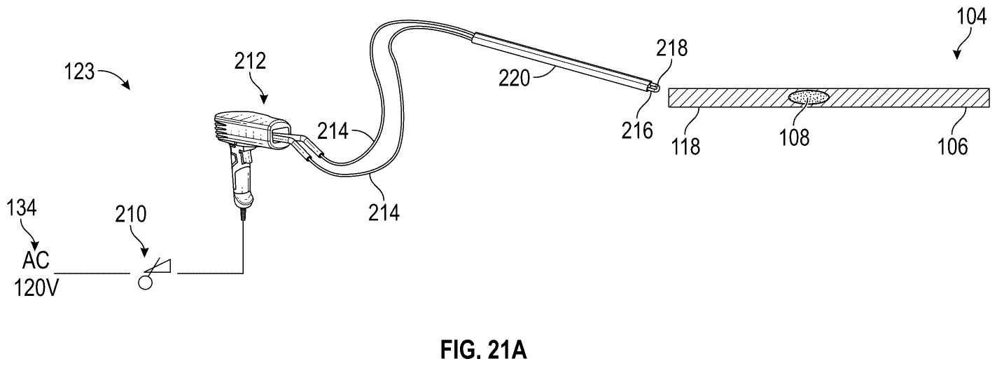

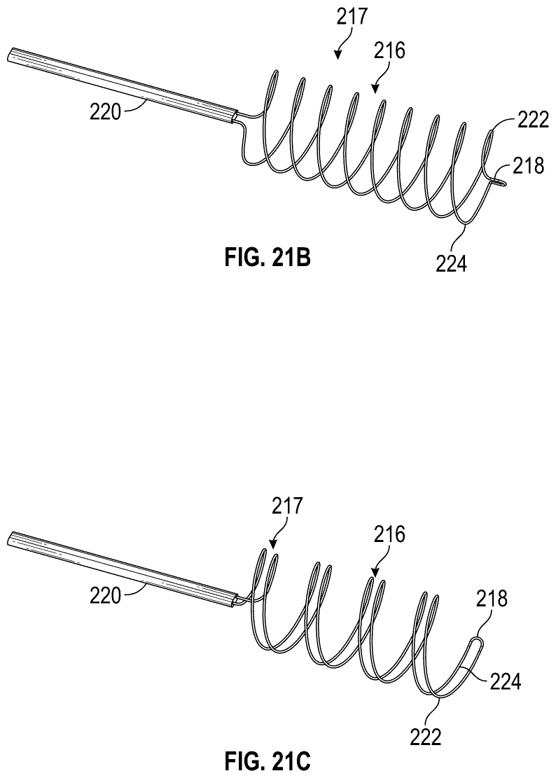

FIG. 21A illustrates a thermal thrombectomy system 123 that may include any of the features of the thermal thrombectomy system 122 described herein. The thermal thrombectomy system 123 can be used to perform a thrombectomy. The thermal thrombectomy system 123 can include a power source 134 that may be referred to as an energy source. The power source 134 may at least include a battery (e.g., disposable, rechargeable etc.), wired power source, and/or others. The power source 134 may be external. The power source 134 may deliver 120V AC or another voltage. The thermal thrombectomy system 123 may include a power adjustment unit 210, which may be a dimmer, to adjust the voltage provided by the power source 134. The power source 134 and/or power adjustment unit 210 may be operatively connected to a current delivery unit 212, which may be a plastic welder. The current delivery unit 212 can enable an operator to selecting supply current to wires 214 of the thermal thrombectomy system 123. For example, the current delivery unit 212 may include a trigger, button, switch, knob, and/or other mechanism that the operator may manipulate (e.g., press) to deliver and cease delivery of current to wires 214. The current delivery unit 212 and power adjustment unit 210 may be a single unit. In some variants, the current delivery unit 212, power adjustment unit 210, and/or power source 134 or a power source interface may be incorporated into a single unit.

The wires 214, which may be copper wires, may be operatively connected to a wire 216, e.g., Nitinol wire, that cuts and/or detaches thrombi. A current of electricity may be delivered to the wire 216, causing the wire 216 to rise in temperature (e.g., heat). The wire 216 may be disposed inside a sheath 220, which may also be referred to as a sleeve, catheter, and/or tube, that may insulate blood vessels from the heat of the wire 216. The wires 214 may extend into the sheath 220 to operatively connect to the wire 216. An end 218 (e.g., looped end) of the wire 216 way extend from a distal end of the sheath 220. The wire 216 may be heated such that the exposed heated looped end 218 may be used to penetrate into a thrombus 108 while the portion of the wire 216 disposed in the sheath 220 is insulated from the wall 118 of the vein 104 or artery.

As illustrated in FIG. 21B, the wire 216 may be advanced distal of the sheath 220 to deploy (e.g., expand) a coil 217. The coil 217 may self-expand once out of the sheath 220. The coil 217 may include a first coil 222 and a second coil 224 that are connected together. The first coil 222 and the second coil 224 may be overlaid on each other. The first coil 222 and the second coil 224 may be positioned coaxially. The coils of the first coil 222 and the second coil 224 can be positioned such that the coils of the first coil 222 are positioned between the coils of the second coil 224. The coils of the second coil 224 may be positioned between the coils of the first coil 222. The coils of the first coil 222 may be equidistantly spaced between the coils of the second coil 224. The coils of the second coil 224 may be equidistantly spaced between the coils of the second coil 224. The coils of the first coil 222 and the second coil 224 may spiral around a longitudinal axis of the coil 217 with the coils of the first coil 222 disposed (e.g., equidistantly disposed) between adjacent coils of the second coil 224. The first coil 222 and the second coil 224, in some variants, do not touch until joining at the looped end 218. The gaps in the proximal-distal direction between the coils of the first coil 222 and the second coil 224 may be equal. The proximal end of the first coil 222 may be connected to one of the wires 214 and the proximal end of the second coil 224 may be connected to the other of the wires 214. The distal ends of the first coil 222 and the second coil 224 may join together at the looped end 218. The looped end 218 may extend in a distal direction. The looped end 218 may be positioned on the longitudinal axis of the coil 217.

As illustrated in FIG. 21C, the wire 216 may be advanced distal of the sheath 220 to deploy (e.g., expand) a coil 217. The coil 217 may self-expand once out of the sheath 220. The coil 217 may include a first coil 222 and a second coil 224. The first coil 222 and the second coil 224 may be positioned coaxially. The coils of the first coil 222 and the second coil 224 can be positioned such that the coils of the first coil 222 are positioned between the coils of the second coil 224. The coils of the second coil 224 may be positioned between the coils of the first coil 222. The coils of the first coil 222 may be parallel with the coils of the second coil 224. The coils of the first coil 222 may be proximate the coils of the second coil 224. A coil of the first coil 222 may be closer in a proximal-distal direction to a first adjacent coil of the second coil 224 compared to a second adjacent coil of the second coil 224. The first coil 222 and the second coil 224, in some variants, do not touch until joining at the Looped end 218. The proximal end of the first coil 222 may be connected to one of the wires 214 and the proximal end of the second coil 224 may be connected to the other of the wires 214. The distal ends of the first coil 222 and the second coil 224 may join together at the end 218 (e.g., looped end). The looped end 218 may extend in the spiral direction of the adjoining portions of the first coil 222 and second coil 224.

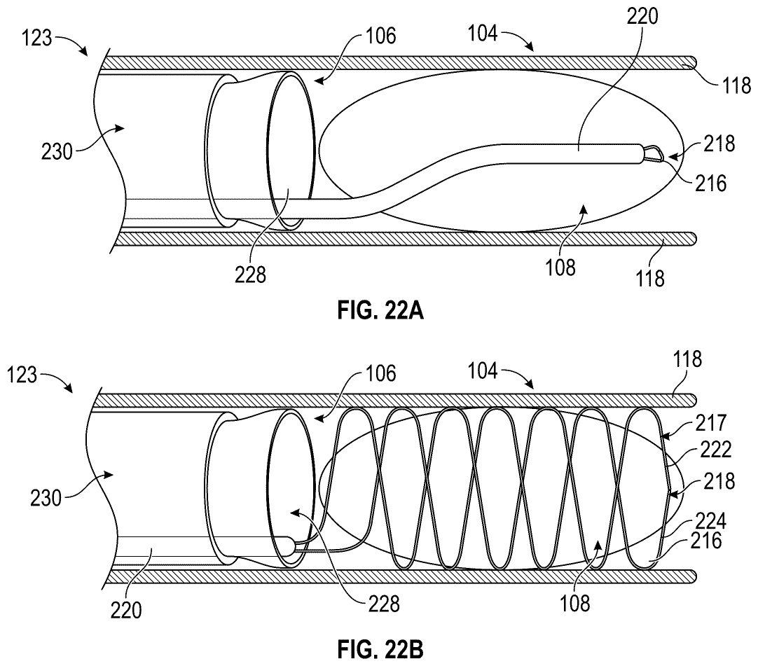

FIGS. 22A and 22B illustrate a method of performing a thrombectomy with the thermal thrombectomy system 123 having the wire 216 and sheath 220 described in reference to FIGS. 21A-21C. As illustrated, the sheath 220 may be routed to a thrombus 108. The wire 216 may be disposed in the sheath 220. As illustrated in FIG. 22A, the sheath 220 and/or wire 216 may be delivered to proximate the thrombus 108 by way of a catheter 228 and/or catheter 230. For example, the catheter 230 may be navigated through the vascular system to proximate (e.g., proximally proximate) the thrombus 108. The catheter 228 may be advanced through the catheter 230 to proximate (e.g., proximally proximate) the thrombus 108. The sheath 220 with the wire 216 disposed therein may be advanced through the catheter 228 to distal the catheter 228 and/or catheter 230. The looped end 218 of the wire 216 may be disposed distally outside the sheath 220. The wire 216 may be heated. The sheath 220 and wire 216 therein may be advanced together distally such that the sheath 220 and wire 216 penetrate the thrombus 108. The exposed heated looped end 218 may ease penetration through the thrombus 108. The heated looped end 218 may Soften and/or emulsify the thrombus, which can include one or more proteins (e.g., collagen) and/or other thrombus forming elements. The sheath 220 may insulate the blood vessel, catheter 228, and/or catheter 230 from the heat of the wire 216.

As illustrated in FIG. 22B, the sheath 220 may be retracted proximally to deploy (e.g., unsheathe, uncover, expand, etc.) the coil 217 of the wire 216. In some variants, the wire 216 may be advanced such that the coil 217 is positioned distally outside the sheath 220 to deploy (e.g., unsheathe, uncover, expand, etc.). The coil 217 may be unsheathed in the thrombus 108. The coil 217 may self-expand outside the sheath 220. The expansion of the coil 217 may fragment the thrombus 108. The heat of the coil 217 may soften and/or emulsify the thrombus 108. In some variants, the coil 217 may be deployed and sit in the thrombus 108 to soften and/or emulsify the thrombus 108, which can include one or more proteins (e.g., collagen) and/or other thrombus forming elements. The wire 216 may be retracted proximally to fragment the thrombus 108, detach the thrombus 108 from the wall 118 of the vein 104, and/or pull the thrombus 108 into the catheter 228 for removal. In some variants, the wire 216 may be rotated to fragment the thrombus 108 and/or detach the thrombus 108 from the wall 118 of the vein 104. In some variants, the catheter 228 and/or catheter 230 can aspirate the thrombus 108.

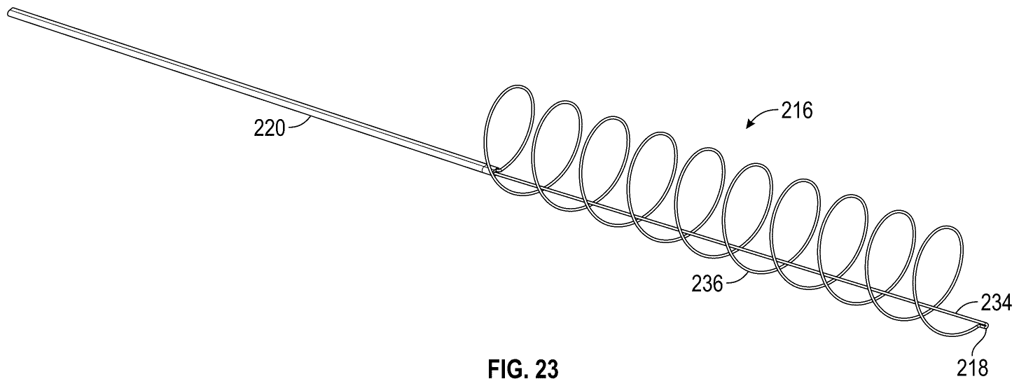

FIG. 23 illustrates an example sheath 220 and wire 216 that can be used with the thermal thrombectomy systems described herein. The wire 216 may include a coil 236. The coil 236 may be a helical coil. The proximal end of the coil 236 may be operatively connected (e.g., by way of a wire) to an energy source (e.g., source of electrical energy). The wire 216 may include a straight portion 234. The straight portion 234 may extend the length of the coil 236. The straight portion 234 may extend through the coil 236. The straight portion 234 may extend through the coil 236 within an inner periphery of the coil 236. The proximal end of the straight portion 234 may be operatively connected (e.g., by way of a wire) to the energy source (e.g., source of electrical energy). The coil 236 and the straight portion 234 may join together at a looped end 218. As described herein, the looped end 218 may extend distally from the sheath 220 and/or a catheter to ease penetration into a thrombus. The looped end 218 may extend in the longitudinal direction of the straight portion 234 and/or coil 236. Energy, such as electrical energy, can be applied to the wire 216 to heat the wire 216 to soften and/or emulsify the thrombus, which can include one or more proteins (e.g., collagen) and/or other thrombus forming elements, which may ease penetration of the thrombus, detaching (e.g., scraping) the thrombus from the walls of the blood vessel, and/or fragmenting the thrombus. The sheath 220 may insulate the patient's anatomy from the heat of the wire 216. The wire 216 may be placed (e.g., retracted) in the sheath 220, and when the wire 216 is distally advanced to move the coil 236 out of the sheath 220, the coil 236 may automatically deploy (e.g., expand). The wire 216 may be placed in a catheter, and when the wire 216 is distally advanced to move the coil 236 out of the catheter, the coil 236 may automatically deploy (e.g., expand). The coil 236 may self-expand outside the sheath 220.

In use, the coil 236 may be deployed prior to penetrating the thrombus. The coil 236 may be advanced distally and/or rotated to thread into thrombus. In some variants, the sheath 220 and wire 216 may penetrate the thrombus prior to the coil 236 being deployed. With the sheath 220 and wire 216 in the thrombus, the sheath 220 may be retracted to deploy the coil 236 in the thrombus. In some variants, the distal end of the sheath 220, with the wire 216 disposed in the sheath 220, may be advanced distal of the thrombus. The wire 216 may be advanced distally relative to the thrombus and/or the sheath 220 may be retracted relative to the wire 216 to move the coil 236 outside the sheath 220 to deploy. With the coil 236 deployed, the wire 216 may be retracted in a proximal direction and/or rotated into the thrombus.

Different portions of the wire 216 may be heated. For example, in some variants, the straight portion 234, looped end 218, and/or coil 236 may be heated. In some variants, the straight portion 234, looped end 218, and/or coil 236 may be insulated (e.g., include an insulating material, insulating coating, insulating cover, etc.). In some variants, the inner periphery of the coil 236 may be heated and the outer periphery of the coil 236 may be insulated.

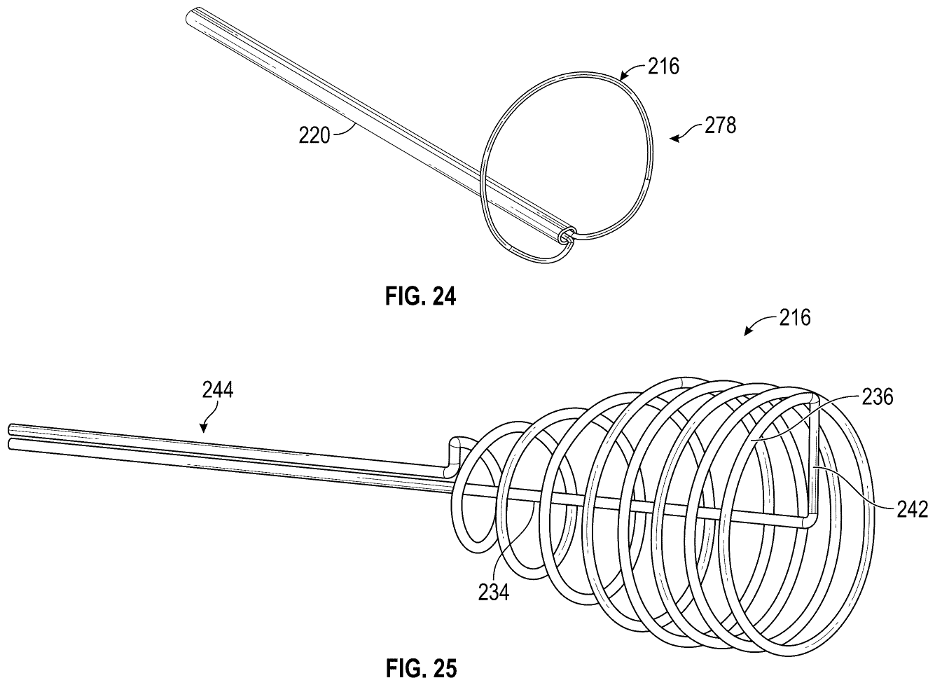

FIG. 24 illustrates an example sheath 220 and wire 216 that can be used with the thermal thrombectomy systems described herein. The wire 216 may form a lasso 278, which may also be called a hoop or loop. The lasso 278 may be retracted into and/or deployed from a sheath 220, which may be a catheter. When retracted, the wire 216 may be fully disposed in the sheath 220 or a looped end may extend beyond the distal end of the sheath 220, which may facilitate penetration of a thrombus even when the wire 216 is sheathed. One side of the lasso 278 may be operatively connected (e.g., connected by way of a first wire) to an energy source (e.g., source of electrical energy) and the other side of the lasso 278 may be operatively connected (e.g., connected by way of a second wire) to the energy source. Energy, such as electrical energy, can be applied to the wire 216 to heat the wire 216 to soften and/or emulsify the thrombus, which can include one or more proteins (e.g., collagen) and/or other thrombus forming elements, which may ease penetration of the thrombus, detachment (e.g., scraping) of the thrombus from the walls of the blood vessel, and/or fragmenting the thrombus. The wire 216 may be distally advanced out of the sheath 220 and/or the sheath 220 may be proximally retracted relative to the wire 216 to deploy the lasso 278. The wire 216 may be distally advanced various distances relative to the sheath 220 and/or the sheath 220 may be proximally retracted various distances relative to the sheath 220 to form various sizes of lassos (e.g., lassos of different diameters). For example, when operating in a blood vessel of a larger diameter, the wire 216 may be advanced distally farther relative to the sheath 220 to form a lasso 278 having a larger diameter compared to when operating in a blood vessel of a smaller diameter. The lasso 278 may self-expand outside the sheath 220.

In use, the lasso 278 may be deployed prior to penetrating the thrombus. The lasso 278 may be advanced distally and/or rotated into thrombus. In some variants, the sheath 220 and wire 216 may penetrate the thrombus prior to the lasso 278 being deployed. With the sheath 220 and wire 216 in the thrombus, the sheath 220 may be retracted to deploy the lasso 278 in the thrombus. In some variants, the distal end of the sheath 220, with the wire 216 disposed in the sheath 220, may be advanced distal of the thrombus. The wire 216 may be advanced distally relative to the thrombus and/or the sheath 220 may be retracted relative to the wire 216 to move the lasso 278 outside the sheath 220 to deploy. With the lasso 278 deployed, the wire 216 may be retracted in a proximal direction and/or rotated into the thrombus, which may capture the thrombus for extraction, fragment the thrombus, and/or detach the thrombus from the vessel walls.

FIG. 25 illustrates an example wire 216 that can be used with the thermal thrombectomy systems described herein. The wire 216 may be retracted into and/or extended distally from a sheath, as described herein. The wire 216 may include a coil 236. The coil 236 may be a helical coil. The coil 236 may be tapered in a proximal to distal direction or a distal to proximal direction. The coil 236 may be tapered from an intermediate portion to the proximal portion such that the intermediate portion has a diameter that is larger than the diameter of the proximal portion. The diameter of the coil 236 may be the same from the intermediate portion to the distal portion. The proximal portion of the coil 236 may have a diameter that is smaller than the distal portion of the coil 236. The proximal end of the coil 236 may be operatively connected (e.g., by way of a wire) to an energy source (e.g., source of electrical energy).

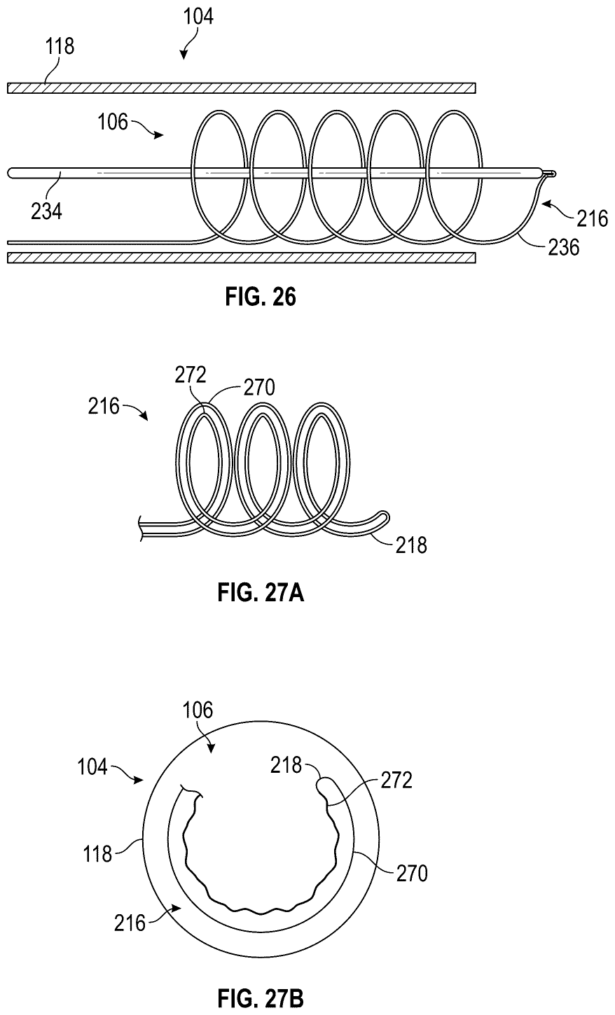

The wire 216 may include a straight portion 234. The straight portion 234 may extend the length of the coil 236. The straight portion 234 may extend through the coil 236, which may include extending coaxially along a longitudinal axis of the coil 236. The straight portion 234 may extend through the coil 236 within an inner periphery of the coil 236. The proximal end of the straight portion 234 may be operatively connected (e.g., by way of a wire) to the energy source (e.g., source of electrical energy). The coil 236 and the straight portion 234 may join together distally (e.g., the distal ends of the coil 236 and the straight portion 234 may be joined together). For example, a transverse portion 242 (e.g., perpendicular portion) may connect the distal end of the straight portion 234 with the distal end of the coil 236. Energy, such as electrical energy, can be applied to the wire 216 to heat the wire 216 to soften and/or emulsify the thrombus, which can include one or more proteins (e.g., collagen) and/or other thrombus forming elements, which may ease penetration of the thrombus, detaching (e.g., scraping) the thrombus from the walls of the blood vessel, and/or fragmenting the thrombus. The sheath 220 may insulate the patient's anatomy from the heat of the wire 216 not unsheathed. The wire 216 may be placed (e.g., retracted) into the sheath 220, and when the wire 216 is distally advanced to move the coil 236 out of the sheath 220, the coil 236 may automatically deploy (e.g., expand). The wire 216 may be placed in a catheter, and when the wire 216 is distally advanced to move the coil 236 out of the catheter, the coil 236 may automatically deploy (e.g., expand). The coil 236 may self-expand outside the sheath 220. FIG. 26 illustrates a variation to the wire 216 and sheath 220 described in reference to FIG. 25, wherein the straight portion 234 may be insulated (e.g., coated with an insulating material, sheathed in an insulating covering, etc.)

In use, the coil 236 may be deployed prior to penetrating the thrombus. The coil 236 may be advanced distally and/or rotated to thread into thrombus. In some variants, the sheath 220 and wire 216 may penetrate the thrombus prior to the coil 236 being deployed. With the sheath 220 and wire 216 in the thrombus, the sheath 220 may be retracted to deploy the coil 236 in the thrombus. In some variants, the distal end of the sheath 220, with the wire 216 disposed in the sheath 220, may be advanced distal of the thrombus. The wire 216 may be advanced distally relative to the sheath 220 and/or the sheath 220 may be retracted relative to the wire 216 to move the coil 236 outside the sheath 220 to deploy. With the coil 236 deployed, the wire 216 may be retracted in a proximal direction and/or rotated into the thrombus, which may capture the thrombus for extraction, fragment the thrombus, and/or detach the thrombus from the vessel walls.

Different portions of the wire 216 may be heated. For example, in some variants, the straight portion 234, looped end 218, and/or coil 236 may be heated. In some variants, the straight portion 234, looped end 218, and/or coil 236 may be insulated (e.g., include an insulating material, insulating coating, insulating cover, etc.). In some variants, the inner periphery of the coil 236 may be heated and the outer periphery of the coil 236 may be insulated.

FIGS. 27A and 27B illustrate an arrangement for the wire 216 that can be used with the thermal thrombectomy systems described herein. The wire 216 can include an outer coil 270 and/or an inner coil 272. The inner coil 272 can be disposed inside of the outer coil 270 (e.g., within an inner periphery of the outer coil 270). The inner coil 272 and the outer coil 270 may be parallel relative to each other. The proximal end of the outer coil 270 may be operatively connected (e.g., by way of a wire) to an energy source (e.g., source of electrical energy). The proximal end of the inner coil 272 may be operatively connected (e.g., by way of another wire) to the energy source (e.g., source of electrical energy). The distal ends of the outer coil 270 and the inner coil 272 may be joined together at a looped end 218. The looped end 218 may extend in the direction of the distal ends of the outer coil 270 and the inner coil 272. The looped end 218 may extend distally along a central axis of the outer coil 270 and/or inner coil 272. The outer coil 270 and the inner coil 272 may be deployed from and/or retracted into a sheath as described herein. Energy, such as electrical energy, can be applied to the wire 216 to heat the wire 216 to soften and/or emulsify the thrombus, which can include one or more proteins (e.g., collagen) and/or other thrombus forming elements, which may ease penetration of the thrombus, detaching (e.g., scraping) the thrombus from the walls of the blood vessel, and/or fragmenting the thrombus.

In use, the outer coil 270 and the inner coil 272 may be deployed prior to penetrating the thrombus. The outer coil 270 and the inner coil 272 may be advanced distally and/or rotated to thread into thrombus. In some variants, the sheath 220 and wire 216 may penetrate the thrombus prior to the outer coil 270 and the inner coil 272 being deployed. With the sheath 220 and wire 216 in the thrombus, the sheath 220 may be retracted to deploy the outer coil 270 and the inner coil 272 in the thrombus. In some variants, the distal end of the sheath 220, with the wire 216 disposed in the sheath 220, may be advanced distal of the thrombus. The wire 216 may be advanced distally relative to the sheath 220 and/or the sheath 220 may be retracted relative to the wire 216 to move the outer coil 270 and the inner coil 272 outside the sheath 220 to deploy. With the outer coil 270 and the inner coil 272 deployed, the wire 216 may be retracted in a proximal direction and/or rotated into the thrombus, which may capture the thrombus for extraction, fragment the thrombus, and/or detach the thrombus from the vessel walls.

Different portions of the wire 216 may be heated. For example, in some variants, the outer coil 270 and/or the inner coil 272 may be heated. In some variants, the outer coil 270 and/or the inner coil 272 may be insulated (e.g., include an insulating material, insulating coating, insulating cover, etc.). For example, as illustrated in FIG. 27B, the inner coil 272 may be heated and the outer coil 270 may be unheated and/or insulated to protect the wall of the blood vessel.

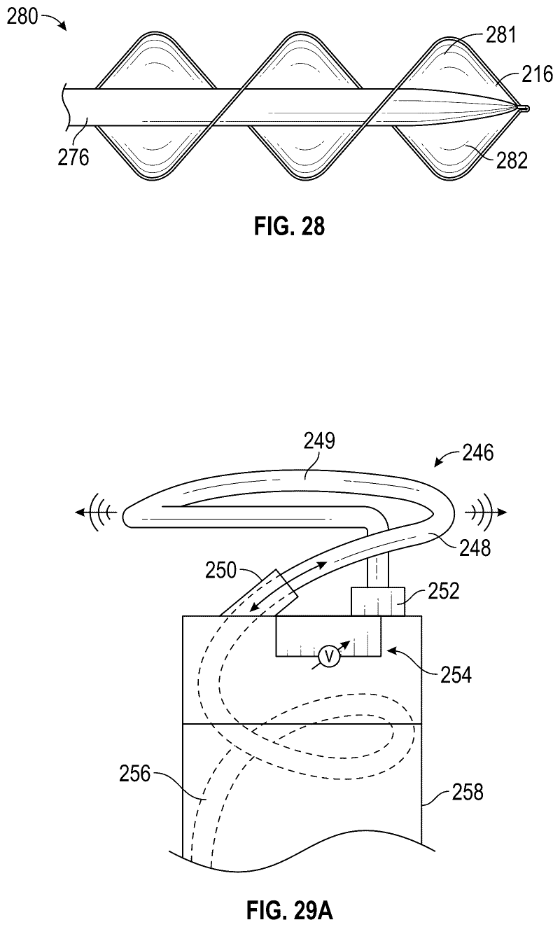

FIG. 28 illustrates a cutting device 280 that may be used with the thermal thrombectomy systems described herein. The cutting device 280 may include a catheter 276 that may be routed to a thrombus, which may include being advanced along a guide wire. The catheter 276 may include a plurality of flutes (e.g., vanes), which may include a first flute 281 and/or a second flute 282. The first flute 281 and/or second flute 282 may spiral along at least a portion (e.g., distal portion) of the catheter 276. The wire 216 may be disposed along the edges of the first flute 281 and/or second flute 282. The ends of the wire 216 may be connected to an energy source (e.g., source of electrical energy). Energy, such as electrical energy, can be applied to the wire 216 to heat the wire 216 to soften and/or emulsify the thrombus, which can include one or more proteins (e.g., collagen) and/or other thrombus forming elements, which may ease penetration of the thrombus, detaching (e.g., scraping) the thrombus from the walls of the blood vessel, and/or fragmenting the thrombus. In some variants, the cutting device 280 may be disposed inside of another catheter or sheath, which may cause the first flute 281 and/or second flute 282 to be collapsed. The first flute 281 and/or second flute 282 may self-expand when moved outside of the catheter.

In use, the first flute 281 and second flute 282 may be deployed prior to penetrating the thrombus. The first flute 281 and second flute 282 may be advanced distally and/or rotated to thread into thrombus. In some variants, the first flute 281 and second flute 282 may deployed in the thrombus. In some variants, the first flute 281 and second flute 282 may deployed distal to the thrombus and then retracted into the thrombus, which may include rotating.

Different portions of the cutting device 280 may be heated. For example, in some variants, the wire 216, first flute 281, second flute 282, and/or portions of the catheter 276 may be heated. In some variants, the wire 216, first flute 281, second flute 282, and/or portions of the catheter 276 may be insulated (e.g., include an insulating material, insulating coating, insulating cover, etc.).

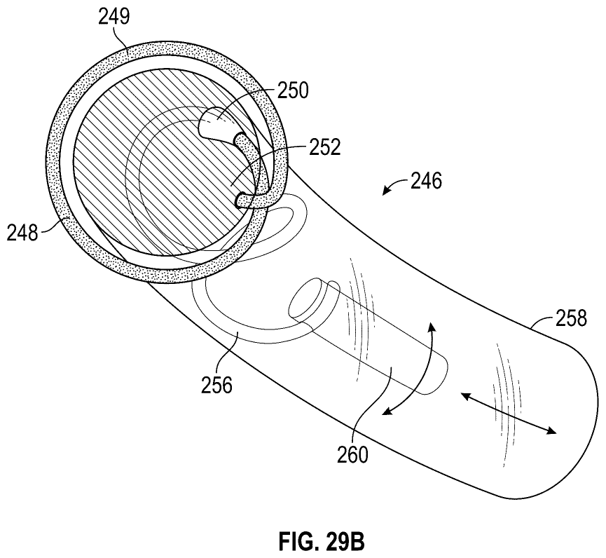

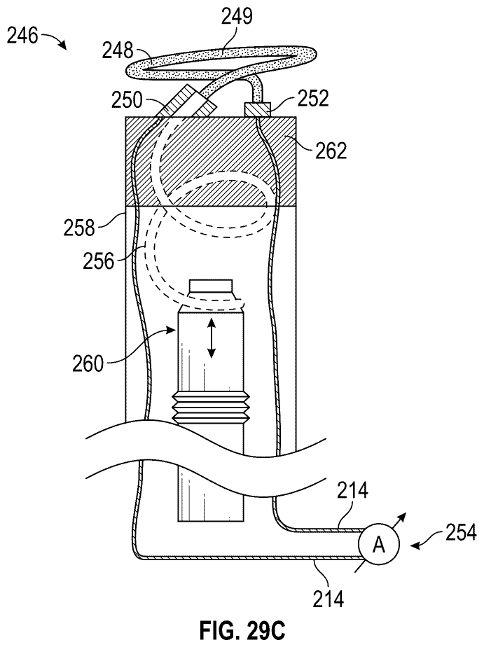

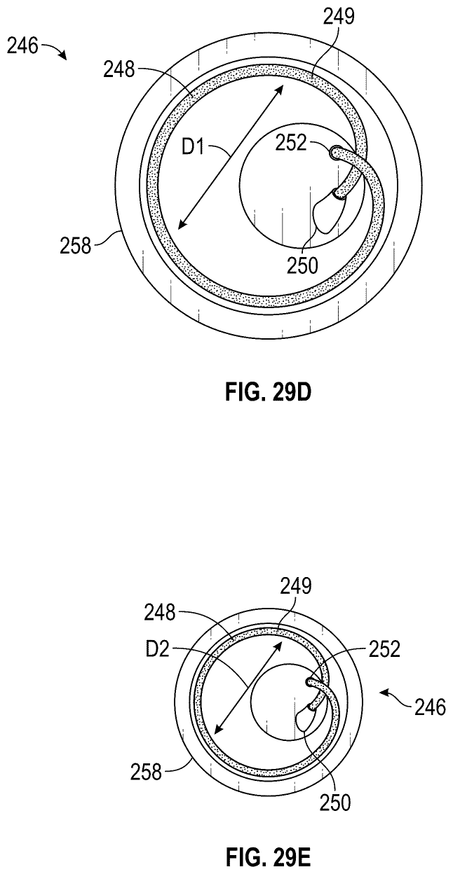

FIGS. 29A-29D illustrate a thermal thrombectomy system 246. The thermal thrombectomy system 246 may include a variable lasso 248, which may also be referred to as a variable loop and/or variable hoop. As illustrated in FIG. 29A, the variable lasso 248 may be formed from a wire 249, which may be made of various materials including Nitinol. An internal portion 256 of the wire 249, which may have a coil shape, may be disposed inside of a catheter 258 (e.g., sheath). The internal portion 256 of the wire 249 may be advanced out of the catheter 258 through a port 250 (e.g., opening, hole, etc.) to increase the size (e.g., diameter) of the variable lasso 248. The internal portion 256 may be retracted into the catheter 258 by way of the port 250 to decrease the size (e.g., diameter) of the variable lasso 248. The port 250 may be disposed on a distal surface of the catheter 258. An end of the variable lasso 248, opposite the port 250, may be fixed to a mount 252. The mount 252 may be disposed on the catheter 258 (e.g., a distal surface of the catheter 258). The distal surface of the catheter 258 may be flat. Energy, such as electrical energy, can be applied to the variable lasso 248 to heat the variable lasso 248 to soften and/or emulsify the thrombus, which can include one or more proteins (e.g., collagen) and/or other thrombus forming elements, which may ease penetration of the thrombus, detaching (e.g., scraping) the thrombus from the walls of the blood vessel, and/or fragmenting the thrombus. For example, the port 250 and/or mount 252 can include electrical contacts that may be connected to a power source 254, which may be variable, to apply energy (e.g., electrical energy) to the variable lasso 248.

As illustrated in FIG. 29B, the proximal end of the internal portion 256 of the wire 249 may be connected to a cable 260. The cable 260 may be rotated in a first direction to advance the internal portion 256 out of the port 250 to increase the diameter of the variable lasso 248. The cable 260 may be rotated in a second direction, opposite the first direction, to retract the 249 back into the catheter 258 by way of the port 250 to decrease the diameter of the variable lasso 248.

As illustrated in FIG. 29C, the electrical contacts of the port 250 and/or mount 252 may be electrically connected to the power source 254 by way of wires 214. The distal end of the catheter 258 may include a rigid tip 262.

As illustrated in FIG. 29D, the variable lasso 248 may be expanded to a diameter DI for a larger vessel, and as illustrated in FIG. 29E, the diameter of the variable lasso 248 may be decreased to a diameter D2, which is less than D1, for a smaller vessel. The diameter of the variable lasso 248 may be set such that the variable lasso 248 is proximate the wall of the vessel to detach a thrombus therefrom.

In use, the distal end of the thermal thrombectomy system 246 may be disposed proximate a thrombus. The diameter of the variable lasso 248 may be set based on the diameter of the vessel. Electrical energy may be applied to the variable lasso 248 to heat the variable lasso 248. The variable lasso 248 may be advanced to soften and/or emulsify the thrombus, which can include one or more proteins (e.g., collagen) and/or other thrombus forming elements to detach the thrombus from the vessel wall and/or fragment the thrombus.

The thermal thrombectomy systems and methods disclosed herein may use oscillating, sharp ends, and/or vibration. The thermal thrombectomy systems and methods disclosed herein may use grounding/aspiration action. The thermal thrombectomy systems and methods disclosed herein may use a wire of uniform or varying diameter. The thermal thrombectomy systems and methods disclosed herein may include a coil within uniform or varying diameter. The thermal thrombectomy systems and methods disclosed herein may liquefy a thrombus without cauterizing blood or vasculature. The thermal thrombectomy systems and methods disclosed herein may allow minimal invasive use of a suction tool. The thermal thrombectomy systems and methods disclosed herein may include a cutting element off axis (away from the vessel wall) to prevent ablation or cutting of the endolumen. The thermal thrombectomy systems and methods disclosed herein may include coils that scrape the blood vessel walls or are radially inset from the blood vessel wall. The thermal thrombectomy systems and methods disclosed herein may be used to cut unwanted tissue in a controlled manner. The thermal thrombectomy systems and methods disclosed herein may be used to cut false lumen. The thermal thrombectomy systems and methods disclosed herein may include coils with lead-in circular, closed loop, or elliptical coils. The thermal thrombectomy systems and methods disclosed herein may be disposed about the entire perimeter of a thrombus and/or inside a vessel wall border the thrombus. The thermal thrombectomy systems and methods disclosed may use a ultrasonic cutter/jackhammer. The thermal thrombectomy systems and methods disclosed herein may include a water jet back toward a cannula and/or pressurized saline. The thermal thrombectomy systems and methods disclosed herein may include coil diameters between 5 and 16 millimeters. The thermal thrombectomy systems and methods disclosed herein may include coil lengths between endothelial cells 120 and aspiration openings 190 millimeters. The thermal thrombectomy systems and methods disclosed herein may include a wire diameter of 25 millimeters.

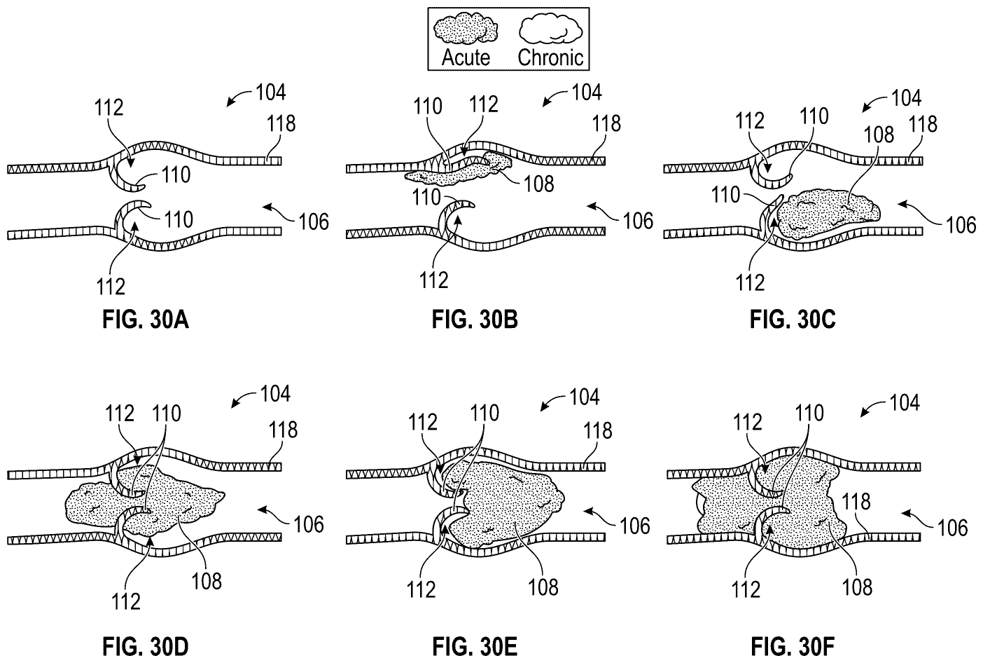

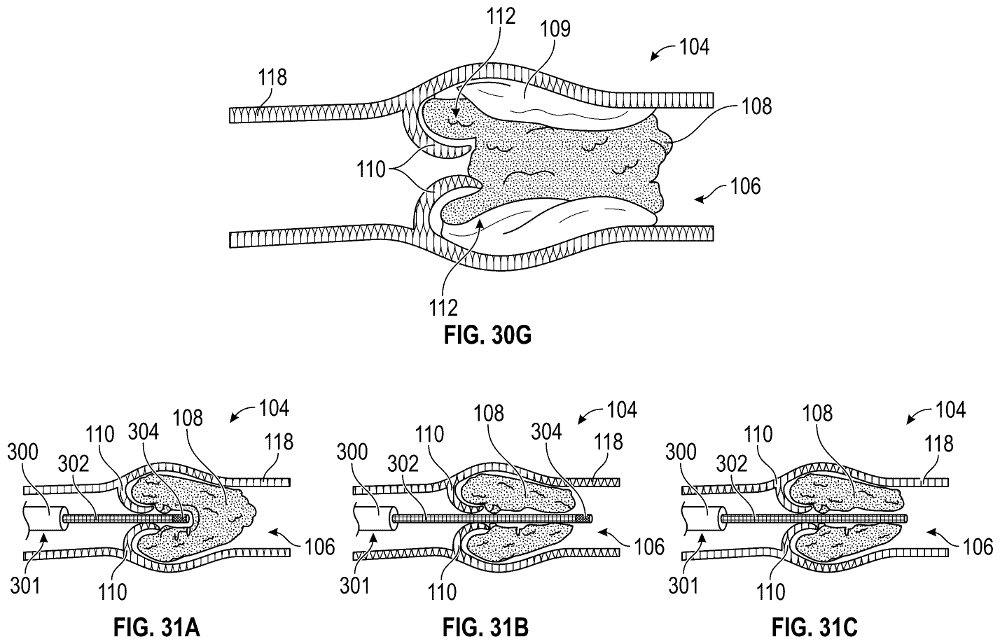

FIGS. 30A-30F illustrate cross-sectional views of veins. As noted elsewhere herein, the systems and methods described herein have been described in reference to veins and thrombi; however, the systems and methods described herein can be used to remove other occlusions in both veins and/or arteries.

FIG. 30A illustrates a cross-sectional view of a vein 104. The vein 104 can include a flow path 106 for blood that is bounded by a wall 118. The vein 104 can include valves 110 that urge the movement of blood within vein 104. The valves 110 can extend radially inward from the wall 118. The valves 110 can extend radially inward at an angle relative to a central longitudinal axis of the vein 104. The vein 104 can include pockets 112 disposed immediately distal of the valves 110.

Thrombi can form at various locations. Thrombi can impair the function of valves 110 (e.g., prevent movement) and/or impede (e.g., partially block, completely block) the flow of blood along the flow path 106 through the vein 104. FIG. 30B illustrates a thrombus 108 forming on one side of a valve 110. FIG. 30C illustrates a thrombus 108 forming in the pocket 112 behind one side of a valve 110. FIG. 30D illustrates a thrombus 108 forming that is disposed both distal and proximal of a valve 110. FIG. 30E illustrates a thrombus 108 forming immediately downstream of a valve 110, which can include filling the pocket 112 immediately distal of the valve 110. FIG. 10F illustrates a thrombus that is disposed both distal and proximal of a valve 110 and in the pocket 112 immediately downstream of the valve 110.

As described herein, a thrombus can change from acute to chronic overtime. FIG. 30G illustrates an acute thrombus 108 that is turning chronic. The thrombus 108 includes some chronic portions 109 (e.g., portions with increased collagen content and/or harder). The chronic portions 109, as illustrated, are disposed on the wall 118.

The systems and methods described herein can at least be used to remove the thrombi illustrated in FIGS. 30B-30G. The systems and methods can use heat to penetrate the thrombi for crossing over the thrombi and/or to detach the thrombi from the wall of the vessel. The thrombi can be captured and retracted proximally for removal. The thrombi can be aspirated. In some variants, a mouth of the aspiration device that aspirates the thrombi can be heated.

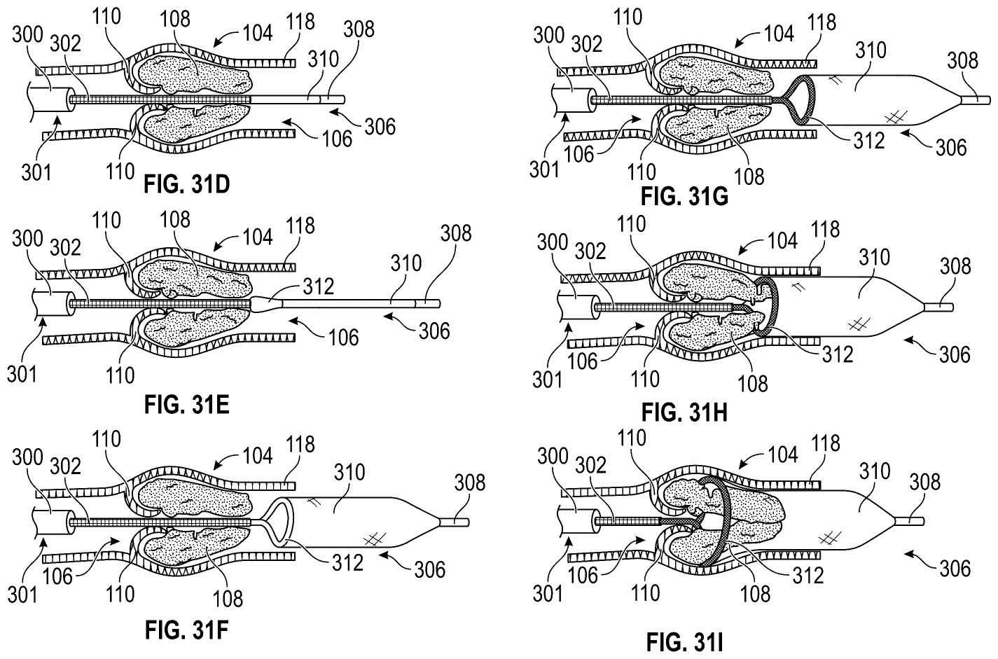

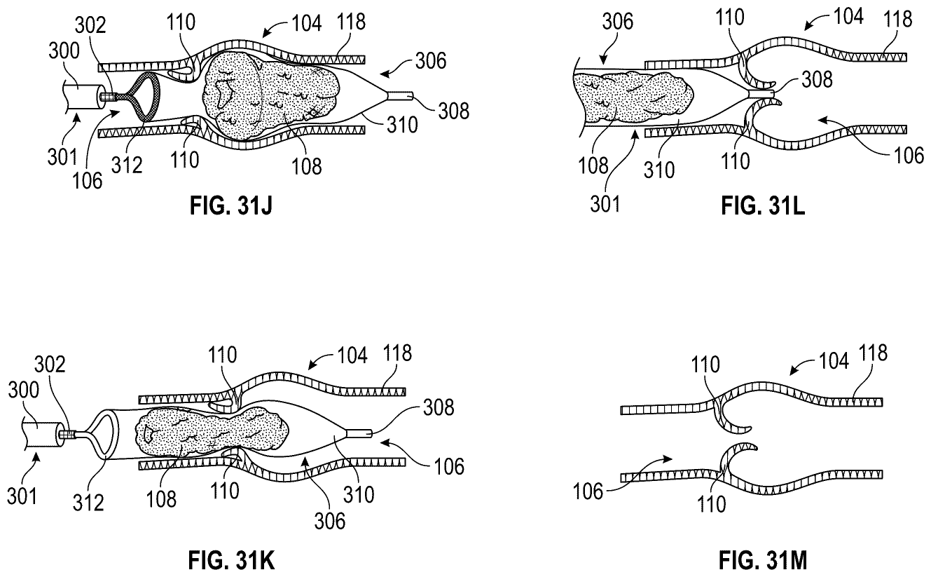

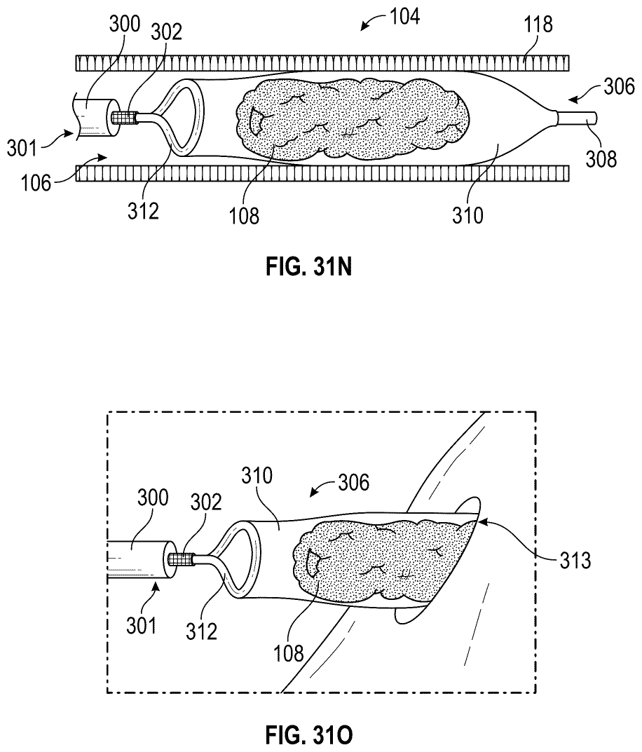

FIGS. 31A-31C illustrate a system 301 (e.g., thermal thrombectomy system, thrombectomy system) with a heated element 304 (e.g., heated member, heated wire, heated guide wire, heated tip, heated end) for penetrating and crossing over a thrombus 108. As illustrated in FIG. 31A, the system 301 can include a catheter 300 (e.g., outer catheter). The outer catheter 300 can be routed through a vein 104 to be proximally positioned relative to the thrombus 108. A heated element 304 can be deployed from within the outer catheter 300 and advanced distally through the thrombus 108. As described herein, the heated element 304 can be heated to at least a temperature described herein. The heated element 304 may be heated directly or indirectly with one or more energy sources, which may at least include heat, radio frequency, laser, electricity (e.g., current), resistive heating, inductive heating, ultrasound, heated fluid, nuclear, and/or others. The heat from the heated element 304 can ease penetration through the thrombus 108. The heat from the heated element 304 can emulsify the thrombus 108, which can include emulsifying a center (e.g., core) of the thrombus 108. The heated element 304 can be disposed inside of a catheter 302 (e.g., inner catheter). The inner catheter 302 can be disposed inside of the outer catheter 300. The inner catheter 302 can be advanced distally with the heated element 304 to cross over the thrombus 108. For example, a distal end of the inner catheter 302 can be advanced with the distal end of the heated element 304 to distal of the thrombus 108, as illustrated in FIG. 31B. With the distal end of the inner catheter 302 distal of the thrombus 108, the heated element 304 can be retracted through the inner catheter 302, leaving the inner catheter 302 in place, as illustrated in FIG. 31C.

FIGS. 31D-31F illustrate the deployment of a capture device 306 (e.g., expandable device) of the system 301. As illustrated in FIG. 31D, the capture device 306 can be advanced distally out of the inner catheter 302 to distal of the thrombus 108. The capture device 306 can include a distal end 308. The distal end 308 can be rigid. In some variants, the distal end 308 can include a heated element, which can replace the heated element 304 to ease penetration of the thrombus 108. The capture device 306 can be advanced distally from the inner catheter 302 to release a bag 310 (e.g., cage, net, mesh, basket), as illustrated in FIG. 31E. The bag 310 can include a loop 312 (e.g., hoop, lasso). The loop 312 can be disposed on a proximal side of the bag 310. The loop 312 and/or bag 310 can be biased radially outward such that, when outside the inner catheter 302, the loop 312 and/or bag 310 expand (e.g., deploy), as illustrated in FIG. 31F, distal of the thrombus 108. The expanded loop 312 and/or bag 310 can, in some variants, contact the wall 118. The expanded loop 312 and/or bag 310 can, in some variants, be disposed proximate the wall 118. The loop 312 can be disposed at an opening into the bag 310.

FIGS. 31G-31I illustrate the retraction of the capture device 306 to capture the thrombus 108. The capture device 306 can include heated elements. For example, the loop 312 can be heated, as illustrated in FIG. 31H. The loop 312 can be heated directly or indirectly with one or more energy sources, which may at least include heat, radio frequency, laser, electricity (e.g., current), resistive heating, inductive heating, ultrasound, heated fluid, nuclear, and/or others. The loop 312 can be heated to a temperature described herein. The entirety of the loop 312 can be heated. Portions of the loop 312 can be heated and others not be heated. An inward facing surface of the loop 312 can be heated. The heated loop 312 and bag 310 can be moved (e.g., retracted, pulled) proximally to the thrombus 108, as illustrated in FIG. 31H. The inner catheter 302 can be moved proximally with the loop 312 and bag 310. The loop 312 can pass between an outer periphery of the thrombus 108 and the wall 118. The loop 312 can separate the thrombus 108 from the wall 118. The heat from the loop 312 can ease separation of the thrombus 108 from the wall 118, which can be referred to as coring the thrombus 108 from the wall 118. The heat from the loop 312 can emulsify the thrombus 108, which can include emulsifying the periphery of the thrombus 108. The thrombus 108 can pass into the bag 310 through the loop 312 as the loop 312 and bag 310 are retracted proximally, as illustrated in FIG. 31I. In some variants, the loop 312 can expand or contract in diameter with the changing diameter of the vein 104 such that the loop 312 follows the wall 118. In some variants, the loop 312 can be a variably-sized loop.

FIGS. 31J-31M illustrate the removal of the thrombus 108 with the capture device 306. As illustrated in FIG. 31J, the loop 312, bag 310, and/or inner catheter 302 can be retracted proximally so that the thrombus 108 is moved through the loop 312 and into the bag 310. The heat from the loop 312 can ease separation of the thrombus 108 from the valve 110, which can include separating without significant damage. With the thrombus 108 disposed inside the bag 310, the temperature of the loop 312 can be lowered. For example, the heating of the loop 312 can stop. The capture device 306 (e.g., loop 312, bag 310, and/or inner catheter 302) and/or outer catheter 300 can be retracted proximally with the thrombus 108 disposed in the bag 310, as illustrated in FIGS. 31K and 31L, to remove the thrombus 108 from the collection site, as illustrated in FIG. 31M. The system 301, which can include the outer catheter 300, inner catheter 302, and/or capture device 306, can be retracted proximally, through the vein 104, as illustrated in FIG. 31N, to remove the thrombus 108 from the patient by way of an aperture 313 (e.g., opening, hole, access location) in the patient, as illustrated in FIG. 310.

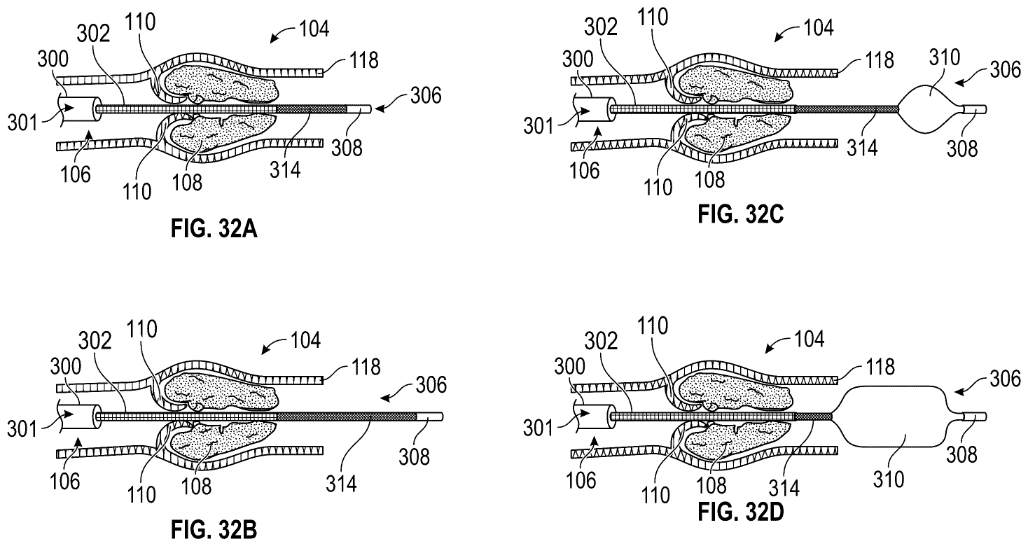

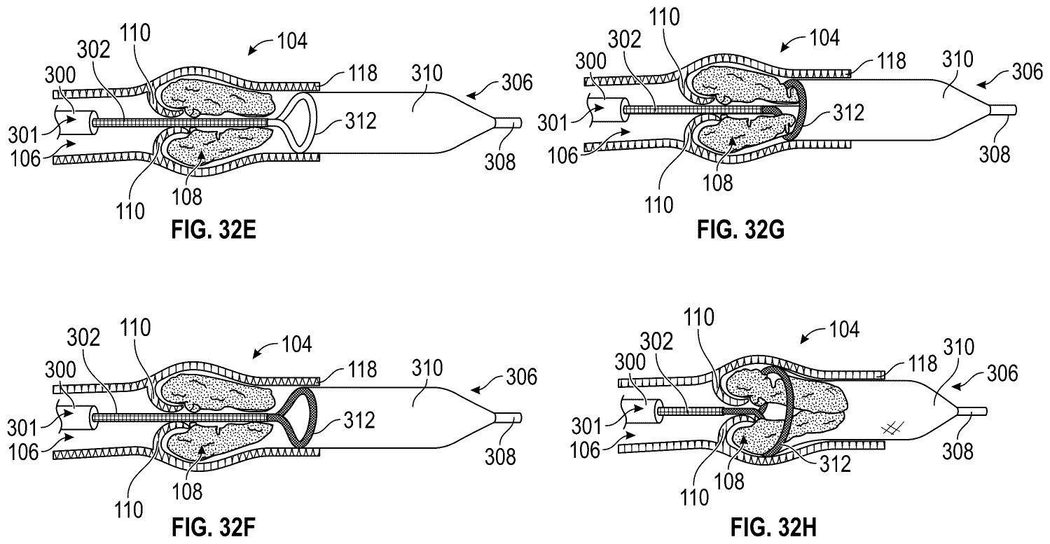

In some variants, the capture device 306 can include a sheath 314, as illustrated in FIG. 32A. The sheath 314 can be disposed over the capture device 306. For example, the sheath 314 can be disposed over the bag 310 and/or loop 312 to prevent expansion of the bag 310 and/or loop 312. The capture device 306 can be advanced distally with the sheath 314 positioned over the bag 310 and/or loop 312 to a position distal of the thrombus 108, as illustrated in FIG. 32B. With the sheathed capture device 306 disposed distally of the thrombus 108, the sheath 314 can be retracted proximally to release the bag 310 and/or loop 312 to expand, as illustrated in FIGS. 32C, 32D, and 32E. The loop 312 can be heated, as illustrated in FIG. 32F. The heated loop 312 can be retracted to the thrombus 108, as illustrated in FIG. 32G. The heated loop 312 can separate the thrombus 108 from the wall 118, which can be eased by the heat from the loop 312, as illustrated in FIG. 32H. The thrombus 108 can then be removed as described herein.

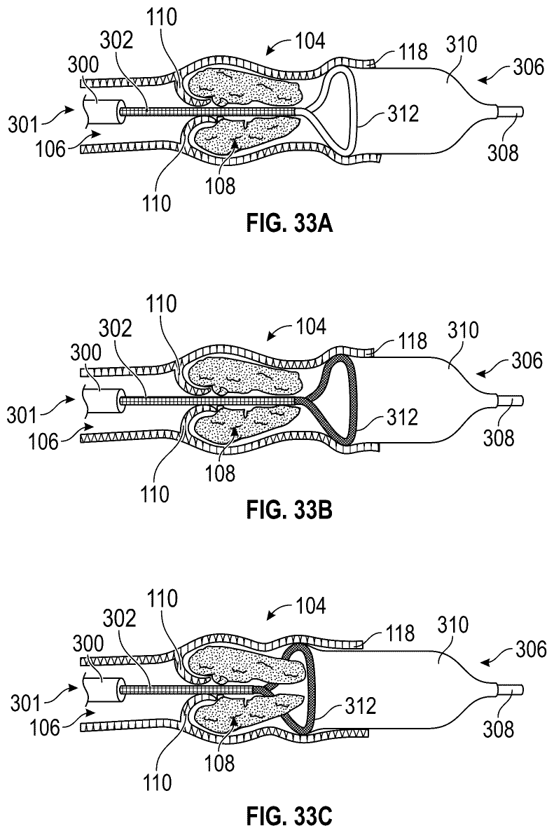

In some variants, the loop 312 and/or bag 310 can include a diameter that is greater than a diameter of the vein 104, as illustrated in FIG. 33A-33C. As illustrated in FIG. 33A, the loop 312 and/or bag 310 can be deployed distally of the thrombus 108. The loop 312 can be heated, as illustrated in FIG. 33B. The heated loop 312 can be retracted proximally to the thrombus 108 to separate the thrombus 108 from the wall 118, as illustrated in FIG. 33C. The heat from the loop 312 can emulsify the thrombus 108, which can include emulsifying the periphery of the thrombus 108. The thrombus 108, separated from the wall 118, can be captured by the bag 310.

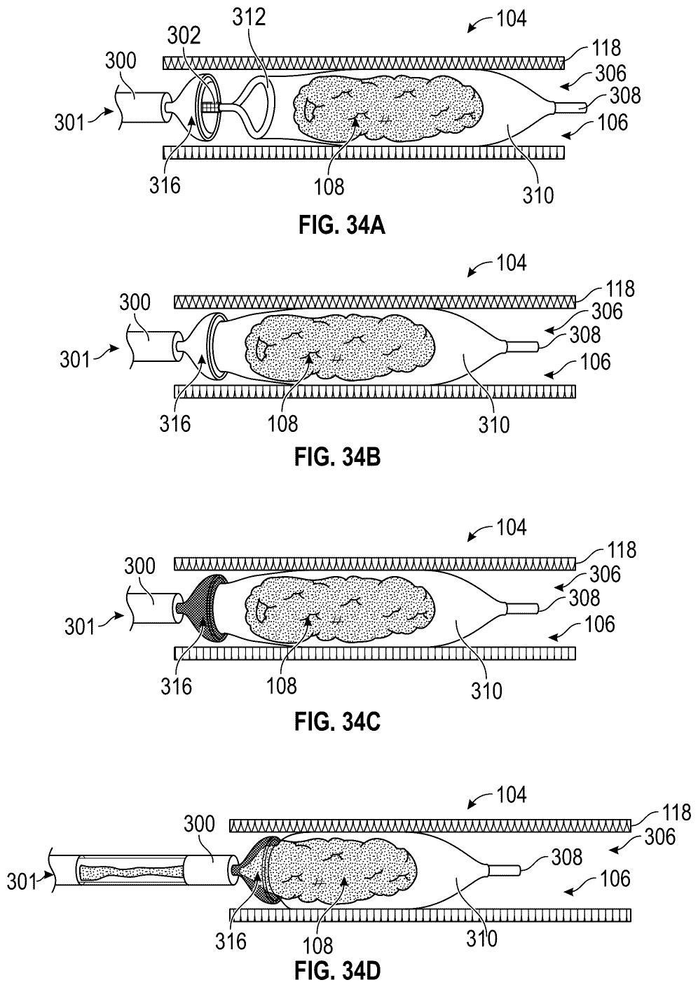

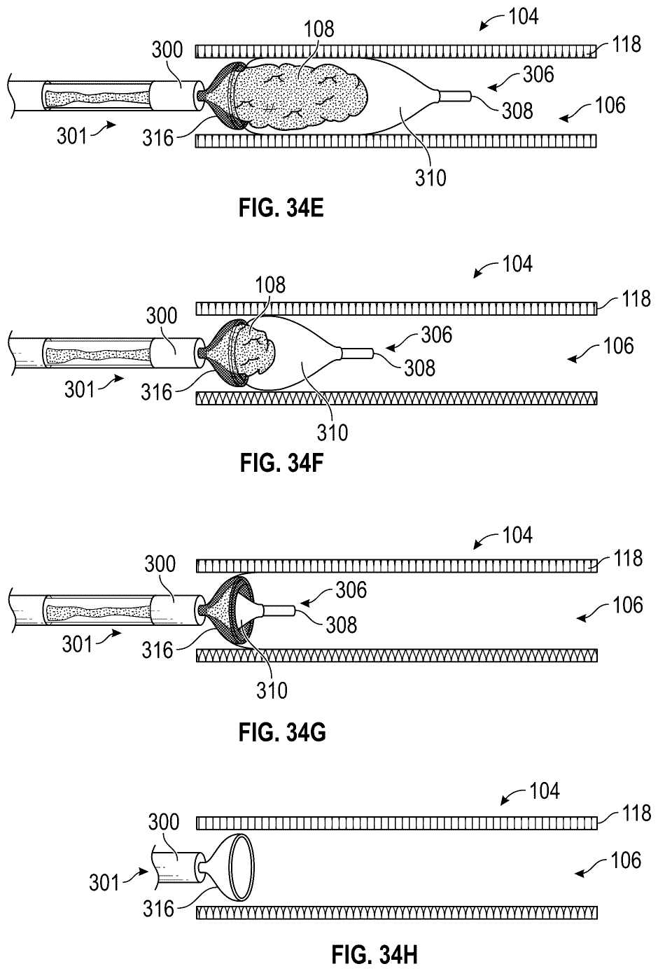

FIGS. 34A-34H illustrate the system 301 with a heated mouth 316 (e.g., funnel, cone, tube, opening) that can be heated to at least the temperatures described herein. The mouth 316 can include a plurality of heated elements, which can include a ring-shaped element, spots, wire(s), band(s), etc. that can be heated. As illustrated in FIG. 34A, the mouth 316 can be disposed at a distal end of the outer catheter 300. In some variants, the mouth 316 can be disposed on a catheter, guide wire, aspiration device, and/or other device that is advanced to a position distal of the distal end of the outer catheter 300. In some variants, the mouth 316 can be a feature of the outer catheter 300. The capture device 306 can be disposed distally of the mouth 316. With the thrombus 108 disposed in the bag 310 of the capture device 306, the capture device 306 (e.g., bag 310, loop 312, and/or inner catheter 302) can be moved proximally toward the mouth 316, as illustrated in FIG. 34B. The mouth 316 can be heated, as illustrated in FIG. 34C. The mouth 316 can be heated using the techniques described herein. The heat from the mouth 316 can emulsify the thrombus 108. For example, as the thrombus 108 approaches and/or contacts the heated mouth 316, the heat from the mouth 316 can soften and/or emulsify the thrombus 108, which can ease entry of the thrombus 108 through the mouth 316 and into the outer catheter 300, as illustrated in FIGS. 34D, 34E, 34F, and 34G. The capture device 306 with the thrombus 108 can be retracted through the mouth 316 and into the outer catheter 300. In some variants, the thrombus 108 can be aspirated through the heated mouth 316 and into the outer catheter 300. With the capture device 306 and thrombus 108 disposed inside the outer catheter 300, the temperature of the mouth 316 can be lowered, as illustrated in FIG. 34H. For example, the heating of the mouth 316 can be stopped.

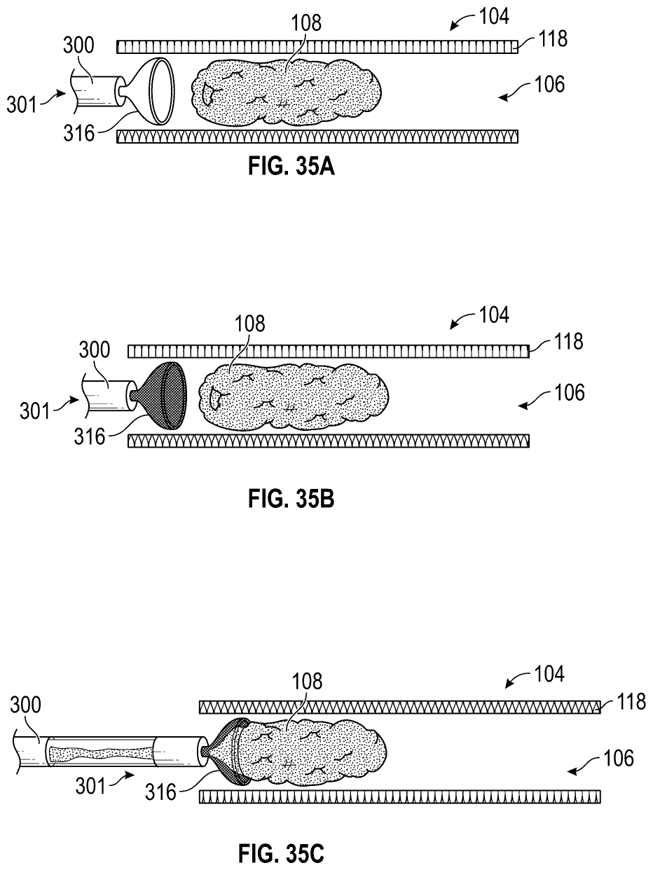

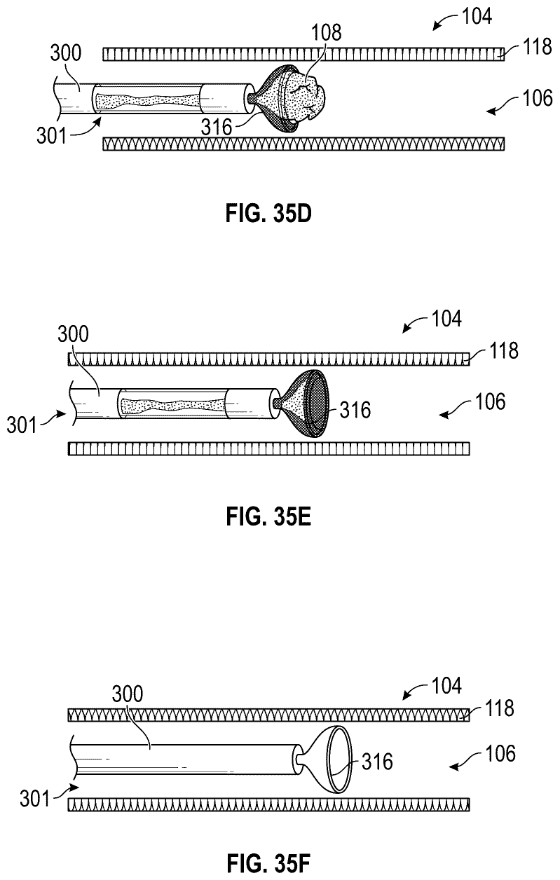

In some variants, the system 301 can omit the capture device 306, as illustrated in FIGS. 35A-35F. As illustrated in FIG. 35A, the mouth 316 can be proximally positioned relative to the thrombus 108. The mouth 316 can be heated, as illustrated in FIG. 35B. The thrombus 108 can be aspirated through the mouth 316 and into the outer catheter 300 for removal as the mouth 316 is advanced distally, as illustrated in FIGS. 35C, 35D, and 35E. The heating of the mouth 316 can soften and/or emulsify the thrombus 108, which can ease aspiration through the mouth 316. The heat from the mouth 316 can ease separation of the thrombus 108 from the wall 118 to facilitate aspiration into the outer catheter 300. With the thrombus 108 aspirated through the mouth 316 and into the outer catheter 300, the temperature of the mouth 316 can be lowered, as illustrated in FIG. 35F. For example, the heating of the mouth 316 can be stopped.

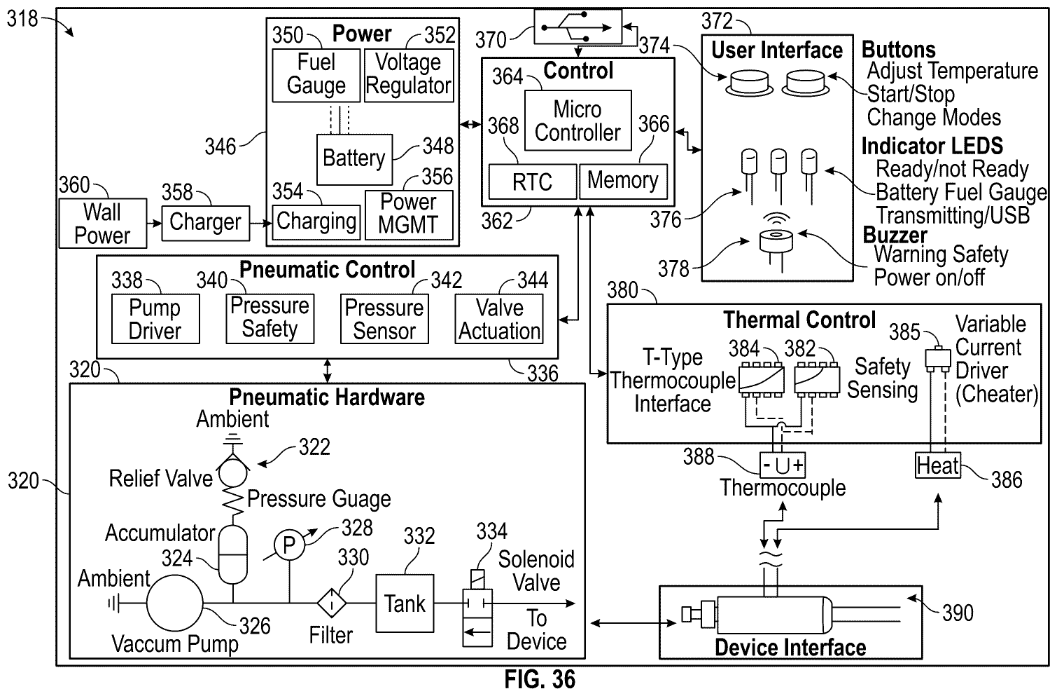

FIG. 36 illustrates a system 318 (e.g., thermal thrombectomy system, thrombectomy system). The system 318 can be used to emulsify and/or aspirate a thrombus, or other occlusion, from a blood vessel. The system 318 can include more or less features than illustrated. The system 318 can include any of the features described in relation to other systems, devices, and/or methods described herein.

The system 318 can include a thermal control system 380 (e.g., temperature control system). The thermal control system 380 can control the temperature (e.g., heating) of a heating element (e.g., loop, mouth, etc.) to emulsify a thrombus. The thermal control system 380 can include a heater 385 (e.g., variable current driver, current driver) that can heat a heating element 386, which can include heating by way of electricity. The heater 385 can heat the heating element 386 to at least temperatures described herein or above to accommodate for heat loss. Wiring for the heater 385 can be routed through a device interface 390 to the heating element (e.g., loop, mouth, etc.) that is disposed inside the blood vessel of the patient. The heating element can be heated to at least the temperatures described herein.

The thermal control system 380 can include a temperature sensor interface 384 (e.g., thermocouple interface, T-type thermocouple interface). The temperature sensor interface 384 can interface with one or more temperature sensors 388 (e.g., thermocouple(s)). The temperature sensor 388 can sense the temperature at or proximate the heating element (e.g., loop, mouth, etc.) and/or at or proximate the interface between the heating element and the thrombus disposed inside the blood vessel of the patient. Wiring for the temperature sensor 388 can be routed through a device interface 390 to or proximate the heating element (e.g., loop, mouth, etc.). The thermal control system 380 can modulate the heat of the heating element based on temperatures and/or pressures sensed at the heating element and/or interface between the heating element and the thrombus.

The system 318 can include a pneumatic system 320 (e.g., aspiration system). The pneumatic system 320 can be used to aspirate a thrombus and/or other occlusion from the blood vessel of the patient. The pneumatic system 320 can include a pump 326 (e.g., vacuum pump), accumulator 324, relief valve 322, pressure gauge 328, filter 330, tank 332, and/or valve 334 (e.g., solenoid valve 334). The tank 332 can receive aspirated material (e.g., thrombi and/or other occlusive material). The system 318 can include a gauge to indicate the fill level of the tank 332, which can include a gauge of the user interface 372. The pneumatic system 320 can be operatively coupled to the device interface 390 to facilitate aspiration.

The system 318 can include a pneumatic control system 336 (e.g., aspiration control system). The pneumatic control system 336 can include a pump driver 338. The pump driver 338 can drive the vacuum pump 326. The pneumatic control system 336 can include a pressure sensor 342, which can sense pressures within the thermal control system 380 and/or the blood vessel. The pneumatic control system 336 can include a valve actuation unit 344 that can actuate (e.g., open, close) the solenoid valve 334. The pneumatic control system 336 can include a pressure safety unit 340 that can monitor pressures within the thermal control system 380 and/or the blood vessel. The pressure safety unit 340 can, when certain pressures are detected, initiate a safety protocol (e.g., cease driving the vacuum pump 326 with the pump driver 338).

The system 318 can include a power system 346. The power system 346 can include a battery 348, which can be rechargeable. The power system 346 can include a gauge 350. The gauge 350 can indicate the status of the battery 348 (e.g., percentage charged, etc.). The power system 346 can include a charging interface 354. The charging interface 354 can interface with a cable 358 (e.g., charging cable) that can interface with a power source 360 (e.g., outlet) to charge the battery 348 and/or directly power the system 318. The power system 346 can include a power management unit 356 and/or voltage regulator 352.

The system 318 can include a control system 362. The control system 362 can execute the methods described herein. The control system 362 can include a micro controller 364, real-time clock 368 (e.g., RTC), and/or memory 366. The real-time clock 368 can be used to monitor durations of methods and/or steps of methods described herein (e.g., duration heating element is at a temperature). The real-time clock 368 can be used to identify triggering events for the system 318 to respond to. The system 318 can include a data interface 370 that can facilitate connecting the system 318 to a computing system to communicate data.

The system 318 can include a user interface system 372. The user interface system 372 can include one or more button(s) 374, which can at least be used to adjust temperature of the heating element, start/stop heating and/or aspiration, change modes, open and/or shut valves, power on/off, and/or other adjustments. The user interface system 372 can, in some variants, include dial(s), switch(es), display(s), touchpad(s), touchscreen(s), knob(s), trigger(s), indicator(s), gauge(s), slider(s), and/or other features. The user interface system 372 can include indicator(s) 376 (e.g., indicator lights, such as LEDs). The indicator(s) 376 can visually indicate when the system 318 is ready for operation and/or not ready for operation. The indicator(s) 376 can visually indicate when the system 318 is aspirating and/or heating. The indicator(s) 376 can indicate the charge level of the battery 348. The indicator(s) 376 can indicate when the system 318 is communicating with another computing system (e.g., transmitting data). The indicator(s) 376 can emit warnings. The indicator(s) 376 can emit various colors and/or patterns of light. The user interface system 372 can include a speaker(s) 532 (e.g., buzzer(s)), which can emit audible sounds to communicate warnings, alert of safety issues, and/or emit sounds when various actions are performed (e.g., power on/off). In some variants, the user interface system 372 can include one or more displays, touchscreens, microphones for spoken commands, etc. In some variants, the indicator(s) 376, speaker(s) 378, display, touchscreen, etc. can indicate the status of the system 318, which can at least include indicating when the heating element is being heated, how long the heating element has been at a temperature or above a temperature, a temperature of the heated element, when the system 318 is aspirating, etc.

The system 318, in some variants, can include a wired communication interface and/or a wireless communication interface to communicate with other computing devices. The system 318 can include a processor and/or other hardware to perform the methods described herein.

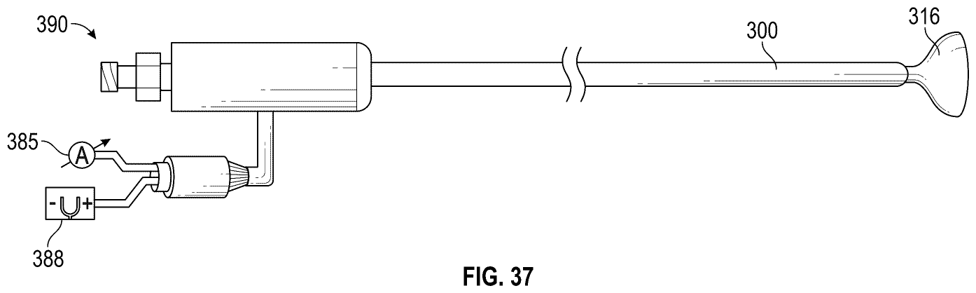

FIG. 37 illustrates the device interface 390. The device interface 390 can be operatively coupled to the outer catheter 300. As described herein, a mouth 316 can be disposed at a distal end of the outer catheter 300. A heater 385 (e.g., variable current driver) can heat the mouth 316 as described herein. The heater 385 can include wiring routed through the outer catheter 300 to heat the mouth 316. A temperature sensor 388 (e.g., thermocouple) can be disposed proximate the mouth 316 to sense temperatures. The temperature sensor 388 can include wiring routed through the outer catheter 300 to position the temperature sensor 388 at the mouth 316.

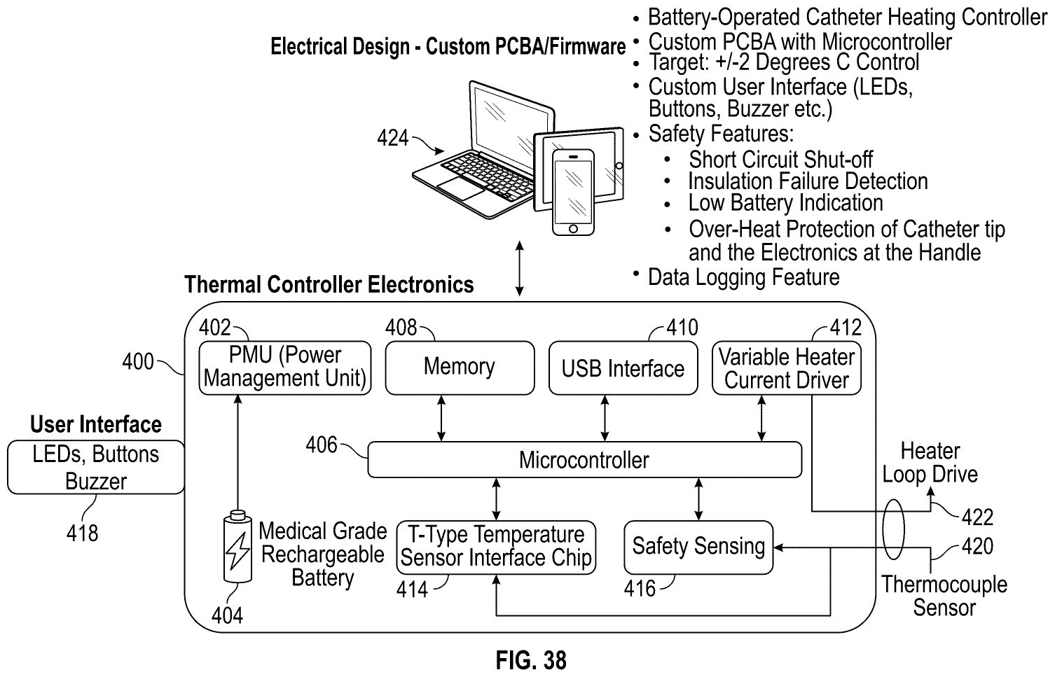

FIG. 38 illustrates a thermal controller system 400 (e.g., thermal thrombectomy control system, thermal thrombectomy system, thrombectomy system, system). The thermal controller system 400 can be used to emulsify a thrombus, or other occlusion in a blood vessel. The thermal controller system 400 can include more or less features than illustrated. The thermal controller system 400 can include any of the features described in relation to other systems, devices, and/or methods described herein. The thermal controller system 400 can include a custom printed circuit board assembly (PCBA) and/or firmware.

The thermal controller system 400 can include a heater 412 (e.g., variable heater current driver). The variable heater current driver 412 can heat a heating element (e.g., loop, mouth, etc.) by way of a conduit 422 (e.g., wire) routed to or proximate a heating element The variable heater current driver 412 can heat the heating element to at least the temperatures described herein.

The thermal controller system 400 can include a temperature sensor interface 414 (e.g., T-type temperature sensor interface chip). The temperature sensor interface 414 can be operatively connected to a temperature sensor (e.g., thermocouple) by way of one or more wires 422 or the like. The temperature sensor can be disposed proximate the heating element, which can include being proximate the interface between the heating element and the thrombus or other occlusion. The thermal controller system 400 can include a safety sensing unit 416, which can monitor the temperature sensed by the temperature sensor. The safety sensing unit 416 can trigger a safety protocol based on the temperature sensed by the temperature sensor, which can include stopping the variable heater current driver 412 from heating the heating element, generating an alert, etc.

The thermal controller system 400 can include a microcontroller 406, data interface 410 (e.g., USB interface), and/or memory 408. The data interface 410 can be used to connect the thermal controller system 400 to a computing system. In some variants, the thermal controller system 400 can include a wireless data interface to enable the thermal controller system 400 to communicate wirelessly with a computing system. The thermal controller system 400 can include a memory 408. The memory 408 can include instructions, protocols, etc. The memory 408 can include a log of data for the thermal controller system 400, which can include temperature data, use data, etc.

The thermal controller system 400 can include a power management unit 402. The thermal controller system 400 can include a battery 404, which can be a rechargeable battery (e.g., medical grade rechargeable battery). The power management unit 402 can manage the current drawn from the battery 404.

The thermal controller system 400 can include a user interface 418, which can be custom. The user interface 418 can be used to control the thermal controller system 400. The user interface 418 can enable the thermal controller system 400 to communicate with the user. The user interface 418 can include light(s) (e.g., LED(s)), button(s), dial(s), switch(es), display(s), touchpad(s), touchscreen(s), knob(s), trigger(s), indicator(s), gauge(s), slider(s), speaker(s), buzzer(s), etc. The user interface 418 can enable a user to adjust the temperature of the heating element (e.g., enable two degrees Celsius adjustments, one degree Celsius adjustments, or even smaller incremental adjustments). The user interface 418 can indicate the charge level of the battery 404.

The thermal controller system 400 can include a short circuit detection system. The thermal controller system 400 can include a short circuit shut-off. The thermal controller system 400 can include insulation failure detection. The thermal controller system 400 can include over-heat protection of the catheter tip and/or electronics at a handle of the thermal controller system 400.

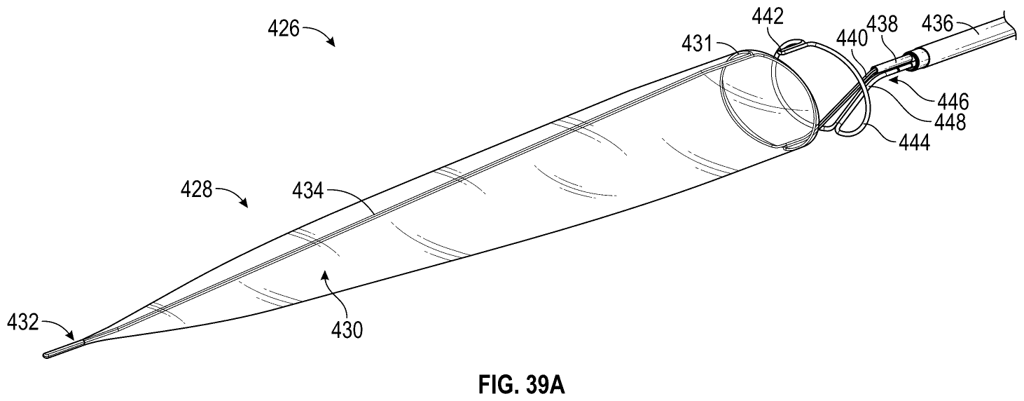

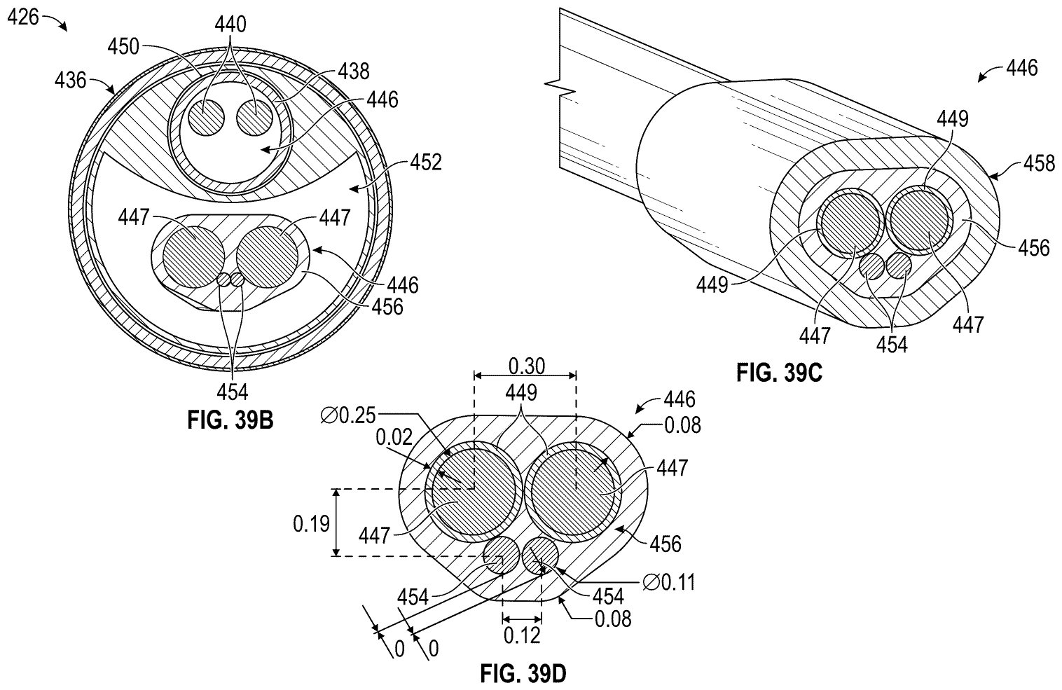

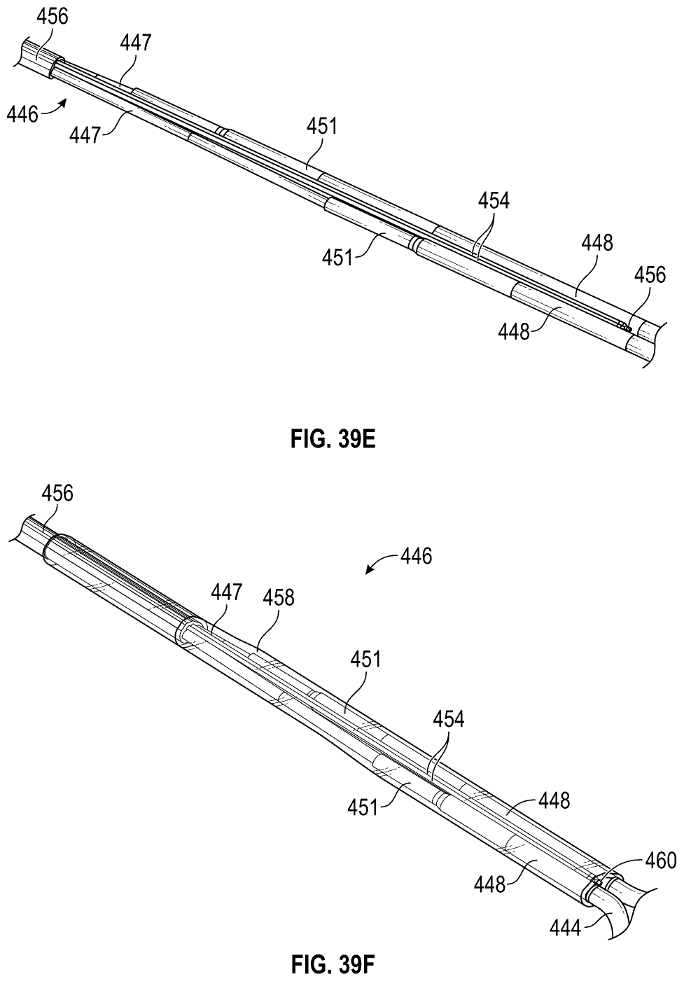

FIG. 39A illustrates a system 426 (e.g., thermal thrombectomy system, thrombectomy system) with a capture device 428 (e.g., expandable device) and a thermal device 446 (e.g., cutting device) deployable from a catheter 436. The capture device 428 can include a bag 430 (e.g., mesh, net, cage, basket) that can capture a thrombus. The bag 430 can, in some variants, be self-expanding. The capture device 428 can include a loop 431 (e.g., hoop, circle, lasso), which can be disposed at the proximal end of the bag 430. The loop 431 can be disposed at (e.g., disposed around) a proximal opening into the bag 430. The loop 431 can be self-expanding. The capture device 428 can include a sheath 438. The sheath 438 can be disposed over the bag 430 and/or loop 431. With the capture device 428 disposed distal of the distal end of the catheter 436, the sheath 438 can be retracted proximally to unsheath the bag 430 and the loop 431 such that the bag 430 and/or loop 431 can expand radially outward. The capture device 428 can include one or more wires 440. The one or more wires 440 can extend through the sheath 438 to the loop 431, which can be formed by the one or more wires 440. A wire segment 434 of the one or more wires 440 can extend between the loop 431 and a distal end 432 of the capture device 428, which can provide rigidity to the bag 430. The distal end 432 can be rigid from the one or more wires 440. In some variants, the one or more wires 440 is a single wire that extends out of the sheath 438, forms one portion (e.g., half) of the loop 431, extends to the distal end 432, returns from the distal end 432 to form the other portion (e.g., half) of the loop 431, and then extends back into the sheath 438. The loop 431 and/or bag 430 can be generally centered around the longitudinal axis of the catheter 436. In some variants, the one or more wires 448 and/or a portion thereof can be heated (e.g., the loop 431, wire segment 434, and/or distal end 432). The bag 430 can be tapered in a proximal-distal direction with the periphery of the distal portion smaller than the proximal portion.

The thermal device 446 (e.g., cutting device) can include a heated loop 444 (e.g., hoop, lasso). The loop 444 can be formed from a wire 448. The wire 448 can be routed distally, form the loop 444, and then be routed proximally. The loop 444 can include a distally extending portion 442. The distally extending portion 442 can be disposed at a top of the loop 444. The distally extending portion 444 can include a u-shaped bend in the wire 448, which can ease distal movement of the loop 444. The wire 448 can be routed distally, form a portion (e.g., half) of the loop, extend distally and loop back proximally to form the distally extending portion 444 (e.g., u-shaped bend), form anther portion (e.g., half) of the loop, and be routed proximally. The loop 444 can be generally centered around the longitudinal axis of the catheter 436. The proximal portions of the wire 448 can be disposed inside of a material (e.g., encased), which can be insulating. The loop 444 and/or loop 431 can be angled distally (e.g., tilted distally). In some variants, the catheter 436 can be routed through another catheter to the thrombus. In some variants, a wire (e.g., guide wire), which can be heated, can be routed through the catheter and not through the catheter 436 to the thrombus.

As described herein, a heated element (e.g., heated wire, heated guide wire) can be advanced distally through the catheter 436 or another catheter to a thrombus or other occlusion. The heated element can be heated and advanced to penetrate the thrombus. The heat can ease penetration of the thrombus by the heated element. The distal end of the catheter 436 can be proximal relative to the distal end of the heated element. The heated element and distal end of the catheter 436 can be advanced to distal of the thrombus. In some variants, the distal end 442 of the thermal device 446 can be advanced distally of the catheter 436 and heated to penetrate the thrombus, instead of using a separate heated element (e.g., heated wire, heated guide wire). The heat from the distal end 442 of the thermal device 446 can ease penetration of the thrombus. The distal end 442 and the distal end of the catheter 436 can be advanced to distal of the thrombus.

The sheathed capture device 428 can be advanced distally out of the catheter 436. The sheath 438 can be retracted proximally to unsheathe the capture device 428 (e.g., the bag 430 and loop 431) to allow the capture device 428 (e.g., the bag 430 and loop 431) to self-expand. The thermal device 446 can be advanced out of the catheter 436 to allow the loop 444 to self-expand. The loop 444 can be heated and retracted proximally to the thrombus. The heated loop 444 can separate the thrombus from the wall of the vessel. The heated loop 444 can emulsify the thrombus (e.g., a periphery of the thrombus). The capture device 428 can be retracted proximally to capture the thrombus detached from the vessel wall. With the thrombus in the bag 430, the capture device 428, thermal device 446, and/or catheter 436 can be retracted proximally out of the patient to remove the thrombus.

FIG. 39B illustrates a cross-section of the system 426. As illustrated, the capture device 428 can be disposed inside of a lumen 450 (e.g., tube, round tube, circular tube) of the catheter 436. The wire 440 of the capture device 428 (e.g., loop 431) can be collapsed (e.g., in a nonexpanded configuration) inside the sheath 438 disposed in the lumen 450. As illustrated, the catheter 436 can include a lumen 452 (e.g., tube, crescent-shaped tube) through which the thermal device 446 can be routed and/or other devices (e.g., guide wire, heated guide wire). The thermal device 446 can include conductive wire(s) 447 that direct heat to the wire(s) 448 that form the loop 444. The thermal device 446 can include temperature sensor wire(s) 454 (e.g., thermocouple wire(s)). The conductive wire(s) 447 and/or temperature sensor wire(s) 454 can be disposed inside of a casing 456 (e.g., insulation, covering), which can be made of a variety of material (e.g., epoxy).

FIG. 39C illustrates a cross-section of the thermal device 446. As illustrated, the wire(s) 447 can include an insulating jacket 449 (e.g., casing, covering). The wire(s) 447 and/or temperature sensor wire(s) 454 can be disposed in a casing 456, which can be disposed inside of a casing 458 (e.g., insulation, covering) that can be made of a variety of materials (e.g., epoxy).

FIG. 39D illustrates a cross-section of the thermal device 446 with example dimensions. The listed dimensions can at least be half to double the sizes listed. In some variants, the insulating jacket 449 can be nominally 0.003 inches thick. The wire(s) 447 can have a diameter that is 0.010 inches in diameter. The temperature sensor wire(s) 454 (e.g., type T or K) can be 0.004 inches in diameter. The wire(s) 448 forming the loop 444 can be 0.020 inches in diameter. The wire(s) 448 can be made of nickel titanium (i.e., Nitinol). As illustrated in FIG. 39E, connections 451 can be used to operatively connect wire(s) 447 and wire(s) 448. The connections 451 can be crimped and/or soldered to operatively couple the wire(s) 447 and wire(s) 448. The temperature sensor wire(s) 454 can extend to a distal end 460 (e.g., sensing end), which can be disposed proximate the junction between the wire(s) 448 forming the loop 444. Sensing can be performed at the distal end 460, which can be disposed distal of the casing 456.

FIG. 39F illustrates a portion of the thermal device 446 within the casing 458, which can be an epoxy. The temperature sensor wire(s) 454 can use a T type thermocouple wire, which can enable precise sensing independent of the Nitinol temperature coefficient. The temperature sensor wire(s) 454 can include a diameter that is 0.0045 inches in diameter. The temperature sensor wire(s) 454 can be insulated (e.g., film insulated). As illustrated, the connections 451 between the wire(s) 447 and the wire(s) 448 can be disposed distal of the casing 456. The connections 451 can be disposed within the casing 458. In some variants, the distal end 460 can be disposed proximate the distal end of the casing 458. In some variants, the distal end 460 can be disposed distal relative to the distal end of the casing 458.

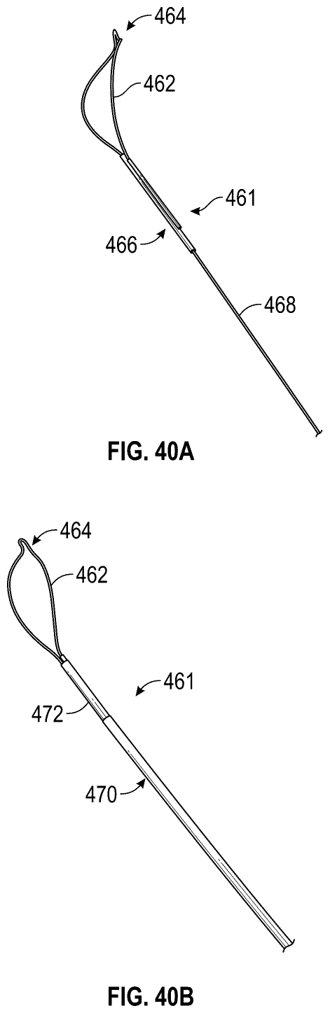

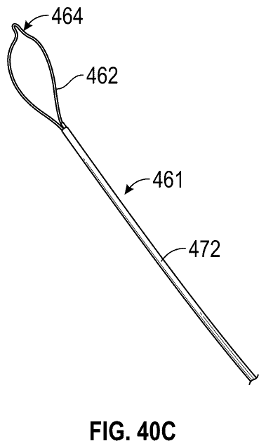

FIGS. 40A-40C illustrate a thermal device 461 (e.g., cutting device). As illustrated in FIG. 40A, the thermal device 461 can include a loop 462 (e.g., hoop, lasso). The loop 462 can include a distal end 464 (e.g., distally extending portion). The distal end 464 can include a u-shaped bend in the wire forming the loop 462, which can ease distal movement of the loop 462. The distal end 464 can be disposed at a top of the loop 444. The wire forming the loop 462 can form one portion (e.g., half) of the loop 462, extend distally and loop back proximally to form the distal end 464 (e.g., u-shaped bend), and form another portion (e.g., half) of the loop 462. The loop 462 can be coupled to one or more stiffeners 468 (e.g., stainless steel stiffener). A covering (e.g., shrink, polytetrafluoroethylene shrink) can be disposed over proximal portions of the wire forming the loop 462, stiffener 468, and/or conductive wires operatively connected to the loop 462 to facilitate heating the loop 462. The loop 462 can be heated to temperatures described herein. The loop 462 can emulsify a thrombus and/or other occlusion in a blood vessel. The loop 462 can detach a thrombus from a wall of the blood vessel. As shown in FIG. 40B, the thermal device 461 can include a sheath 470, which can be varying sizes (e.g., 6Fr). The loop 462 can be retracted into the sheath 470, which can collapse the loop 462. The sheath 470 can be positioned over the loop 462 to impede expansion. The sheath 470 can be retracted proximally to deploy (e.g., expose) the loop 462. The unsheathed loop 462 can automatically expand (e.g., expand radially outward). As illustrated in FIG. 40C, the thermal device 461 can include a casing 472 (e.g., insulation, jacket). The casing 472 can be disposed over the proximal portions of the wire forming the loop 462. The casing 472 can be disposed over at least a portion of the stiffener 468. The casing 472 can be disposed over conductive wires operatively coupled to the loop 462.

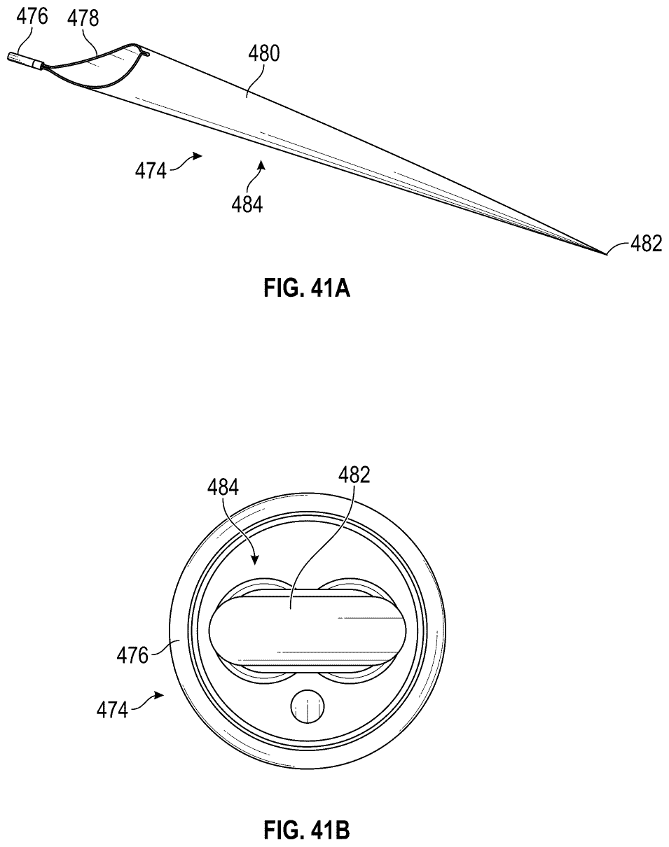

FIGS. 41A and 41B illustrate a system 474 (e.g., thermal thrombectomy system, thrombectomy system, capture device, thermal device) that can be used to remove a thrombus and/or other occlusion from a blood vessel. The system 474 can include a capture device 484 (e.g., expandable device). The capture device 484 can include a loop 478 (e.g., hoop, lasso) that can be heated. The capture device 484 can include a bag 480 (e.g., mesh, net, cage, basket). The bag 480 can be tapered in a proximal-distal direction. The loop 478 can be disposed at a proximal opening into the bag 480. The loop 478 can be integrated with the bag 480. The loop 478, when deployed, can follow the periphery of the bag 480 defining an opening into the bag 480. The system 474 can include a catheter 476. The capture device 484 can be routed through the catheter 476 and deployed therefrom. For example, a distal end 482 of the capture device 484 can be advanced until the bag 480 and loop 478 are distal of the catheter 476, which can free the loop 478 and/or bag 480 to self-expand. In some variants, a wire can extend between the loop 478 and the distal end 482. In some variants, the wire can be heated.

In use, a heated element (e.g., wire, guide wire) can be positioned distally relative to the catheter 476 to interface with a thrombus. The heated element can penetrate the thrombus. The heat from the heated element can ease penetration, which can include emulsifying the thrombus (e.g., central portion of the thrombus). The heated element can cross over (e.g., pass through) the thrombus to a distal side thereof. The catheter 476 with the capture device 484 disposed therein can be advanced with the heated element or after the heated element such that a distal end of the catheter 476 is distal of the thrombus. The capture device 484 can be advanced out of the catheter 476 to deploy (e.g., self-expand) distally of the thrombus. The loop 478 can be heated. The heated loop 478 with the integrated bag 480 can be retracted proximally. The heated loop 478 can interface with the thrombus, which can include emulsifying the thrombus. The heated loop 478 can detach the thrombus from the vessel wall. The thrombus can be captured in the bag 480. The system 474 can be retracted proximally out of the patient with the thrombus in the bag 480. In some variants, the distal end 482 can be heated and advanced distal of the catheter 476 to penetrate the thrombus and facilitate crossover of the thrombus instead of or in combination with a separate heated element.

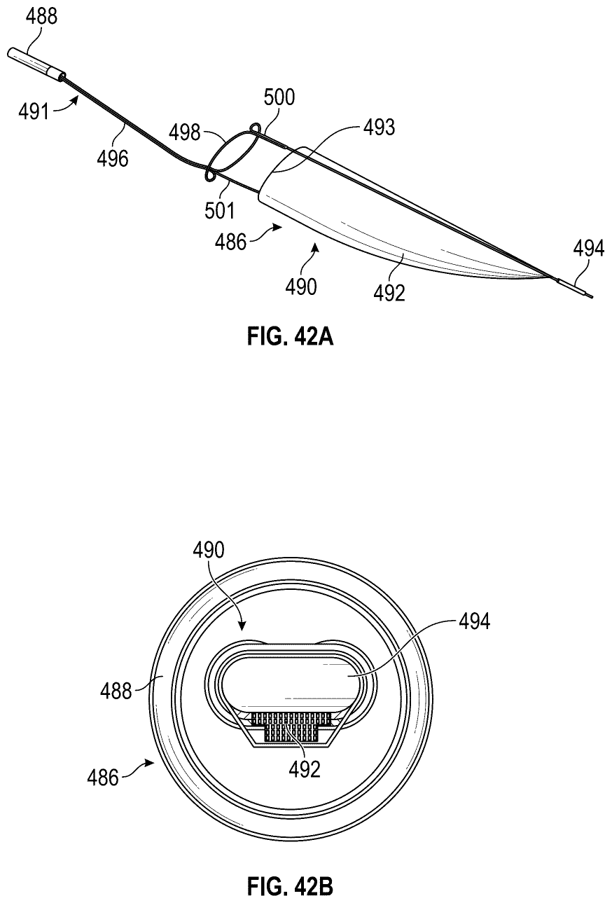

FIGS. 42A and 42B illustrate a system 486 (e.g., thermal thrombectomy system, thrombectomy system, capture device, thermal device) that can be used to remove a thrombus and/or other occlusion from a blood vessel. The system 486 can include a thermal device 491 (e.g., cutting device). The thermal device 491 can include a loop 498 (e.g., hoop, lasso) that can be heated. The thermal device 491 can include one or more wires 496 that can extend to and form the loop 498. The system 486 can include a capture device 490 (e.g., expandable device). The capture device 490 can include a bag 492 (e.g., mesh, net, cage, basket). The bag 492 can be tapered in a proximal-distal direction. The bag 492 can include a consistently sized outer periphery and then taper in a proximal-distal direction. The bag 492 can include a proximal opening, which can include a loop 493 (e.g., wire loop). The thermal device 491 and capture device 490 can be integrated (e.g., coupled, connected) together. For example, a wire segment 500 (e.g., tether, connection, boom, extension boom) can couple the loop 498 of the thermal device 491 to the bag 492 (e.g., loop 493) of the capture device 490. In some variants, the wire segment 500 (boom, extension boom) can extend from the loop 498 to a distal end 494 of the capture device 490 (e.g., bag 492). In some variants, heat from the loop 498 can pass to the capture device 490, which can at least include the bag 492 and/or distal end 494. The distal end 494 can be rigid. The wire segment 500 can be disposed at a top portion of the loop 498. Another wire segment or tether 501 can couple the loop 498 and the bag 492. The another wire segment or tether 501 can be disposed on an opposite side of the loop 498 relative to the wire segment 500.

In use, a heated element (e.g., wire, guide wire) can be positioned distally relative to the catheter 488 to interface with the thrombus. The heated element can penetrate the thrombus. The heat from the heated element can ease penetration, which can include emulsifying the thrombus (e.g., central portion of the thrombus). The heated element can cross over (e.g., pass through) the thrombus to a distal side thereof. The catheter 488 with the capture device 490 and thermal device 491 disposed inside the catheter 488 can be advanced with the heated element or after the heated element such that a distal end of the catheter 488 is distal of the thrombus. The heated element can be retracted proximally back into the catheter 488. The capture device 484 and thermal device 491 coupled to the capture device 484 can be advanced out of the catheter 488 to deploy (e.g., self-expand) distally of the thrombus. The loop 498 can be heated. In some variants, the bag 492 or portions thereof can be heated. The loop 498 and capture device 490 can be coupled together, which can include being coupled (e.g., connected, tethered) together. The thermal device 491 and capture device 490 can be retracted proximally together so that the loop 498 of the thermal device 491 interfaces with the thrombus, which can emulsify the thrombus (e.g., periphery of the thrombus) with the heat from the loop 498. The heat form the loop 498 can help detach the thrombus from the wall of the blood vessel. The thrombus can be captured in the bag 492. With the thrombus captured in the bag 492, the thermal device 491 and capture device 490 can be retracted proximally from the patient to remove the thrombus. In some variants, the distal end 494 can be heated and advanced to distal of the catheter 488 to penetrate the thrombus and facilitate crossover of the thrombus instead of or in combination with a separate heated element.

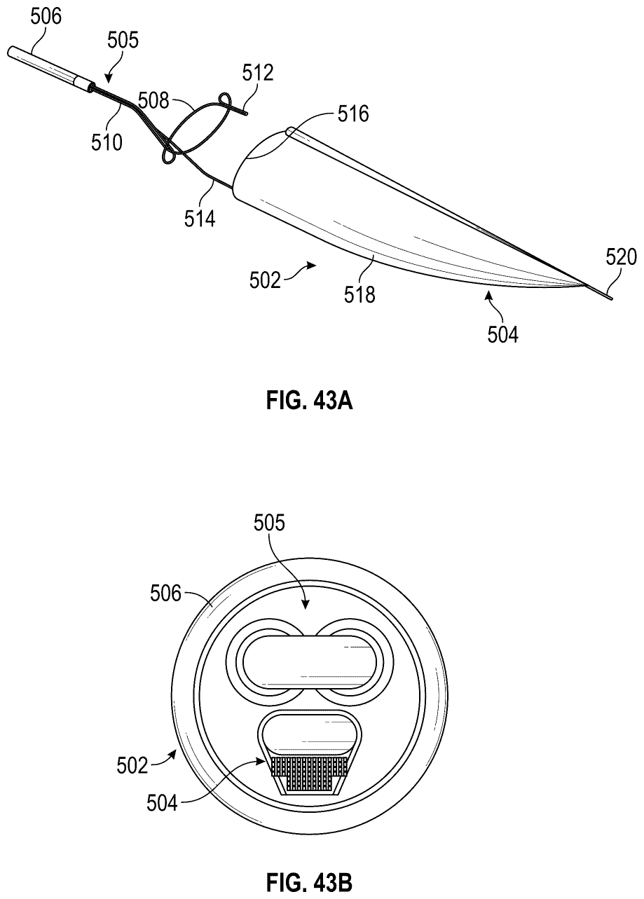

FIGS. 43A and 43B illustrate a system 502 (e.g., thermal thrombectomy system, thrombectomy system, capture device, thermal device) that can be used to remove a thrombus and/or other occlusion from a blood vessel. The system 502 can include a thermal device 505 (e.g., cutting device). The thermal device 505 can include a loop 508 (e.g., hoop, lasso) that can be heated. The loop 508 can include a distal end 512 (e.g., distally extending portion). The distal end 512 can be disposed at a top of the loop 508. The distal end 512 can include a u-shaped bend in a wire 510 forming the loop 508, which can ease distal movement of the thermal device 505. The wire 510 can be routed distally, form a portion (e.g., half) of the loop 508, extend distally and loop back proximally to form the distal end 512, form another portion (e.g., half) of the loop 508, and be routed proximally. The loop 508 can be generally centered around the longitudinal axis of the catheter 506. The proximal portions of the wire 510 can be disposed inside of a material (e.g., encased), which can be insulating.

The system 502 can include a capture device 504. The capture device 504 can include a bag 518 (e.g., mesh, net, cage, basket). The bag 518 can be tapered in a proximal-distal direction. The capture device 504 can include one or more wire(s) 514. The wire 514 can be routed through the catheter 506 to enable a surgeon to maneuver the capture device 504. The wire 514 can form a loop 516 (e.g., hoop, lasso) at the opening into the bag 518. The wire 514 can extend from the loop 516 at the opening of the bag 518 to a distal end 520 of the capture device 504 (e.g., bag 518), which can be referred to as an extension boom and/or boom. The distal end 520 of the capture device 504 can be rigid, which can be provided by the wire 514. The opening into the bag 518 can be generally centered around the longitudinal axis of the catheter 506. In some variants, the wire 514 can be heated.

In use, a heated element (e.g., wire, guide wire) can be positioned distally relative to the catheter 506 to interface with a thrombus. The heated element can penetrate the thrombus. The heat from the heated element can ease penetration, which can include emulsifying the thrombus (e.g., central portion of the thrombus). The heated element can cross over (e.g., pass through) the thrombus to a distal side thereof. The catheter 506 with the thermal device 505 and capture device 504 disposed therein can be advanced with the heated element or after the heated element such that a distal end of the catheter 506 is distal of the thrombus. The thermal device 505 and/or capture device 504 can be advanced out of the catheter 506 to deploy (e.g., self-expand) distally of the thrombus. The loop 508 can be heated. The heated loop 508 can be retracted proximally. The heated loop 508 can interface with the thrombus, which can include emulsifying the thrombus. The heated loop 508 can detach the thrombus from the vessel wall. The thrombus can be captured in the bag 518. The system 502 can be retracted proximally out of the patient with the thrombus in the bag 518. In some variants, the distal end 520 and/or distal end 512 can be heated and advanced distal of the catheter 506 to penetrate the thrombus and facilitate crossover of the thrombus instead of or in combination with a separate heated element.

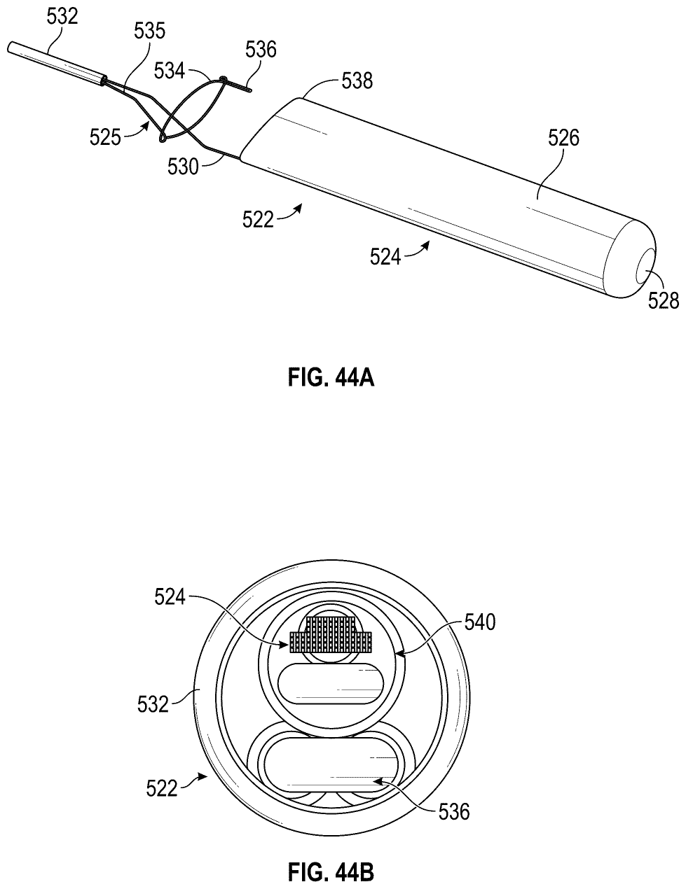

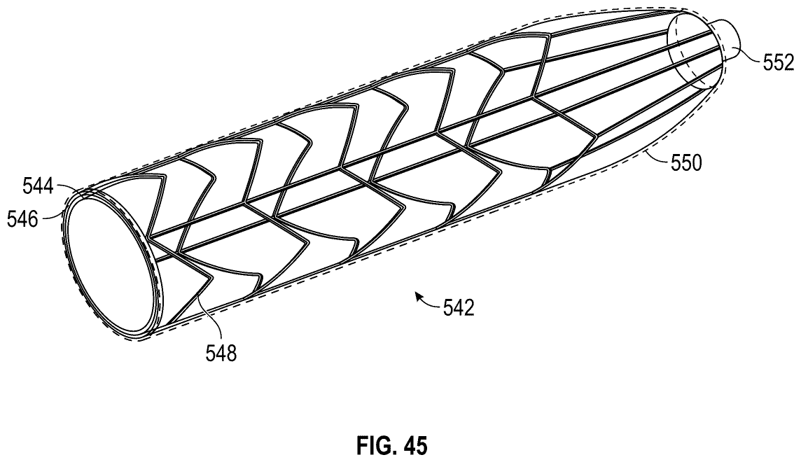

FIGS. 44A and 44B illustrate a system 522 (e.g., thermal thrombectomy system, thrombectomy system, capture device, thermal device) that can be used to remove a thrombus and/or other occlusion from a blood vessel. The system 522 can include a thermal device 525. The thermal device 525 can include a loop 534 (e.g., hoop, lasso) that can be heated. The loop 534 can include a distal end 536 (e.g., distally extending portion). The distal end 536 can be disposed at a top of the loop 534. The distal end 536 can include a u-shaped bend in a wire 535 forming the loop 534, which can ease distal movement of the thermal device 525. The wire 535 can be routed distally, form a portion (e.g., half) of the loop 534, extend distally and loop back proximally to form the distal end 536, form another portion (e.g., half) of the loop 534, and be routed proximally. The loop 534 can be generally centered around the longitudinal axis of the catheter 532. The proximal portions of the wire 535 can be disposed inside of a material (e.g., encased), which can be insulating.

The system 522 can include a capture device 524 (e.g., expandable device). The capture device 524 can include a bag 526 (e.g., mesh, net, cage, basket). The bag 526 can include distal end 528, which can be rounded. The bag 526 can include a generally consistently sized periphery. The capture device 524 can include one or more wire(s) 530. The wire 530 can be routed through the catheter 532 to enable a surgeon to maneuver the capture device 524. The wire 530 can form a loop 538 at the opening into the bag 526. The opening into the bag 526 can be generally centered around the longitudinal axis of the catheter 532. In some variants, the wire 530 can be heated. In some variants, the portion of the wire 530 extending around the opening into the bag 526 forming the loop 538 can be heated. The capture device 524 can include a sheath 540. The sheath 540 can be disposed over the bag 526 to impede expansion of the bag 526. The sheath 540 can be retracted proximally to uncover the bag 526. The unsheathed bag 526 can self-expand. For example, the portion of the wire 530 extending around the opening of the bag 526 to form the loop 538 can self-expand.

In use, a heated element (e.g., wire, guide wire) can be positioned distally relative to the catheter 532 to interface with a thrombus. The heated element can penetrate the thrombus. The heat from the heated element can ease penetration, which can include emulsifying the thrombus (e.g., central portion of the thrombus). The heated element can cross over (e.g., pass through) the thrombus to a distal side thereof. The catheter 532 with the thermal device 525 and capture device 524 disposed therein can be advanced with the heated element or after the heated element such that a distal end of the catheter 532 is distal of the thrombus. The thermal device 525 and/or capture device 526 can be advanced out of the catheter 506. The sheath 540 of the capture device 524 can be retracted. The unsheathed bag 526 can expand (e.g., self-expand). The loop 534 can be heated. The heated loop 534 can be retracted proximally. The heated loop 534 can interface with the thrombus, which can include emulsifying the thrombus. The heated loop 534 can detach the thrombus from the vessel wall. The thrombus can be captured in the bag 526. The system 522 can be retracted proximally out of the patient with the thrombus in the bag 526. In some variants, the distal end 536 can be heated and advanced distal of the catheter 532 to penetrate the thrombus and facilitate crossover of the thrombus instead of and/or in combination with a separate heated element.

FIG. 45 illustrates a capture device 542 (e.g., expandable device) that can be used to emulsify and/or capture a thrombus and/or other occlusion for removal. The capture device 542 can include a bag 548 (e.g., mesh, net, cage, basket, stent). The bag 548 can include a lattice (e.g., matrix) of struts. The bag 548 can be made of a variety of materials (e.g., shape memory alloy such as nickel titanium). The bag 548 can include a distal end 552. The distal end 552 can include a rigid nose (e.g., tail). The distal end 552 can include an annular ring shape. The bag 548 can include a loop 544 (e.g., proximal loop, hoop, lasso) that can define an opening (e.g., proximal opening) into the bag 548. The loop 544 can be collapsible. A heated element 546 (e.g., wire) can be disposed at the loop 544. The heated element 546 can include a loop shape, which can follow the loop 544. The heated element 546 can be disposed along an inner periphery of the loop 544. The heated element 546 can emulsify a thrombus (e.g., emulsify a periphery of the thrombus). The heated element 546 can detach a thrombus from the vessel wall. The heated element 546 can be heated by way of a variety of techniques, which can include electrical current. The loop 544 can included a larger size strut to provide less resistance. In some variants, some current can flow through the bag 548, which can heat the bag 548. The thrombus can be captured in the bag 548. The bag 548 can include a covering 550 (e.g., wrapping, jacket). The covering 550 may not cover the heated element 546, leaving the heated element 546 exposed. The covering 550 may not cover the distal end 552, leaving the distal end 552 exposed. The covering 550 can include be made of a variety of materials, which can at least include expanded polytetrafluoroethylene and/or polytetrafluoroethylene. In some variants, the capture device 542 can include a sheath that can be retracted to expand the capture device 542. The capture device 542 can be deployed from a catheter.

In use, a heated element (e.g., wire, guide wire) can be positioned distally relative to a catheter to interface with a thrombus. The heated element can penetrate the thrombus. The heat from the heated element can ease penetration, which can include emulsifying the thrombus (e.g., central portion of the thrombus). The heated element can cross over (e.g., pass through) the thrombus to a distal side thereof. The catheter with the capture device 542 disposed therein can be advanced with the heated element or after the heated element such that a distal end of the catheter is distal of the thrombus. The capture device 542 can be advanced out of the catheter to deploy (e.g., expand, self-expand) distally of the thrombus. In some variants, the capture device 542 can be disposed in a sheath. The sheath can be retracted to uncover the capture device 542 so that the capture device 542 expands (e.g., self-expands). The heated element 546 can be heated. The capture device 542 can be retracted proximally to interface the heated element 546 with the thrombus, which can emulsify the thrombus (e.g., periphery of the thrombus). The heated element 546 and/or loop 544 can detach the thrombus from the vessel wall. The thrombus can be captured in the bag 548. The capture device 542 can be retracted proximally out of the patient with the thrombus in the bag 548. In some variants, the distal end 552 can be heated and advanced distal of the catheter to penetrate the thrombus and facilitate crossover of the thrombus instead of or in combination with a separate heated element.

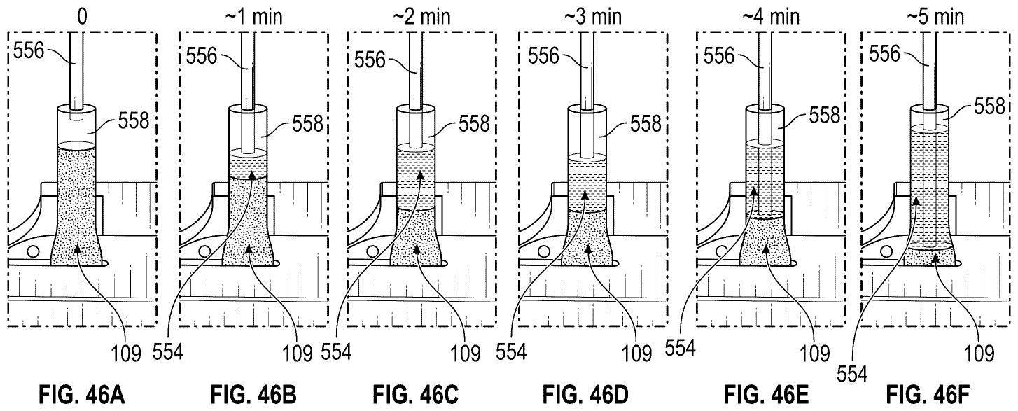

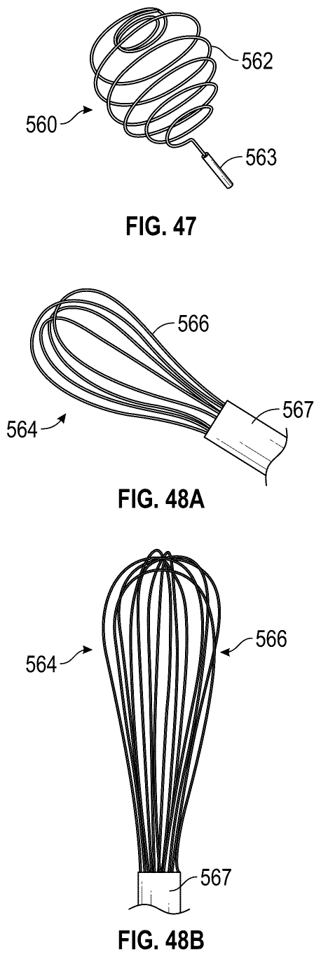

FIGS. 46A-46F illustrate the emulsification of a thrombus during lab testing. FIG. 46A illustrates a chronic thrombus 109 disposed in a tube 558 prior to exposure to heat from a heated element 556. FIG. 46B illustrates the chronic thrombus 109 after about one minute of exposure to the heated element 556 heated to about seventy degrees Celsius. As shown, a portion of the chronic thrombus 109 emulsified after about one minute of exposure, as seen by the emulsified thrombus 554 (e.g., melted, liquefied). FIG. 46C illustrates the chronic thrombus 109 after about two minutes of exposure to the heated element 556 heated to about seventy degrees Celsius. As shown, a larger portion of the chronic thrombus 109 emulsified after about two minutes of exposure, as seen by the emulsified thrombus 554. FIG. 46D illustrates the chronic thrombus 109 after about three minutes of exposure to the heated element 556 heated to about seventy degrees Celsius. As shown, an even larger portion of the chronic thrombus 109 emulsified after about three minutes of exposure, as seen by the emulsified thrombus 554. FIG. 46E illustrates the chronic thrombus 109 after about four minutes of exposure to the heated element 556 heated to about seventy degrees Celsius. As shown, a still larger portion of the chronic thrombus 109 emulsified after about four minutes of exposure, as seen by the emulsified thrombus 554. FIG. 46F illustrates the chronic thrombus 109 after about five minutes of exposure to the heated element 556 heated to about seventy degrees Celsius. As shown, the chronic thrombus 109 is substantially emulsified after about five minutes of exposure, as seen by the emulsified thrombus 554.

FIGS. 47-51 illustrate distal tips (e.g., tips, distal portion, distal ends) of a heating element (e.g., wire, guide wire, guide device, thermal device, cutting device, capture device, and/or other device) that can be used to crossover and/or core a thrombus and/or other occlusion in a blood vessel. Various types of distal tips can be employed, which can include spring loaded, rounded, balloon spaced, rounded wire, coil, and/or auger. For balloon spaced, a balloon catheter can be deployed to space the heating element away from the vessel walls. The distal tips can be self-centering. The distal tips can provide additional surface area to interface with the thrombus for emulsification.

FIG. 47 illustrates a distal tip 560 that can be heated. The distal tip 560 can include a coil 562 (e.g., spiral). The coil 562 can be a coiled wire. The coil 562 can be largest in a radial direction at an intermediate portion (e.g., tapered in size relative to an intermediate portion in a distal direction and proximal direction). The coil 562 can be generally sphere-shaped. The coil 562 can be disposed on a member 563 (e.g., wire, guide wire).

FIGS. 48A and 48B illustrate a distal tip 564 that can be heated. The distal tip 564 can include a plurality of looped wires 566 (e.g., looped members). The looped wires 566 can start at a proximal location, extend distally, and loop back proximally to a proximal location. The looped wires 566 can be disposed on a member 567 (e.g., wire, guide wire). The looped wires 566 can include ends coupled to the member 567.

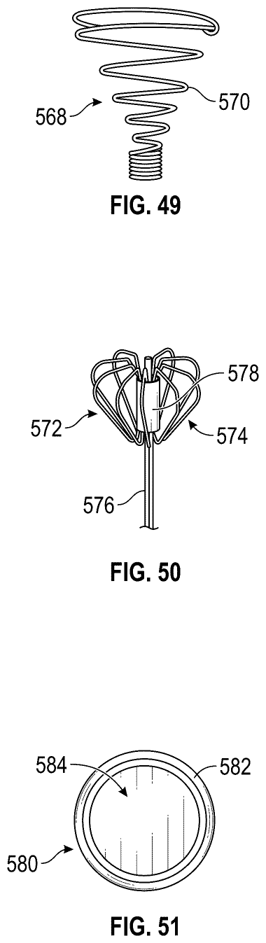

FIG. 49 illustrates a distal tip 568 that can be heated. The distal tip 568 can include a coil 570 (e.g., spiral). The coil 570 can be a coiled (E.g., spiraled) wire. The spiral 570 can be tapered. The spiral 570 can increase in radial size in a distal direction. The distal tip 568 can be disposed on a member (e.g., wire, guide wire).

FIG. 50 illustrates a distal tip 572 that can be heated. The distal tip 572 can include looped wires 574. The looped wires 574 can be coupled to a member 576 (e.g., wire, guide wire). The looped wires 574 can be coupled to the member 576 with a sleeve 578 (e.g., band, covering). The sleeve 578 can be disposed around the member 576. The looped wires 574 can extend out of a distal end of the sleeve 578 and loop back into a proximal end of the sleeve 578.

FIG. 51 illustrates a distal tip 580 that can be heated. The distal tip 580 can include a member 582 (e.g., housing, carrier, tube, catheter). A surface 584 (e.g., button, switch) can be disposed inside the member 582. The surface 584 can be heated. The member 582 can be insulated. The surface 584 can be deployed from within the member 582 to interface with the thrombus. The surface 584 can include a larger contact area to transfer heat to the thrombus. The surface 584 may be deployed with a spring. The surface 584 may be deployed to be flush with surrounding surfaces of the member 582. The surface 584 may be deployed to be distal of surrounding surfaces of the member 582. The surface 584 can be circular.

Thrombi can be heterogeneous. Different regions of a thrombus can emulsify at different rates at different temperatures. In some variants, altering a temperature of a heated element (e.g., wire, loop, mouth, funnel, etc.) based on the characteristics of the thrombus at the interface between the thrombus and the heated element can be beneficial. For example, it may be beneficial to raise a temperature of the heated element when interfacing with a region of a thrombus with chronic characteristics compared to when interfacing with a region of the thrombus with acute characteristics. In some variants, the systems and methods described herein can alter the temperature of the heated element based on the characteristics of the region of the thrombus with which the heated element is interfacing.

The wires and/or heated elements described herein can be made of a variety of materials, such as stainless steel, shape memory alloy (e.g., nickel titanium), etc. In some variants, voltage drop can be protected through grounding wire detection. In some variants, closed-loop techniques and/or artificial intelligence can control (e.g., modulate) the heat (e.g., current) applied by the systems described herein. In some variants, the detection of the type of material, thrombus, and/or occlusion being removed can be detected (e.g., remotely detected) through data, which can include using closed-loop techniques and/or artificial intelligence.

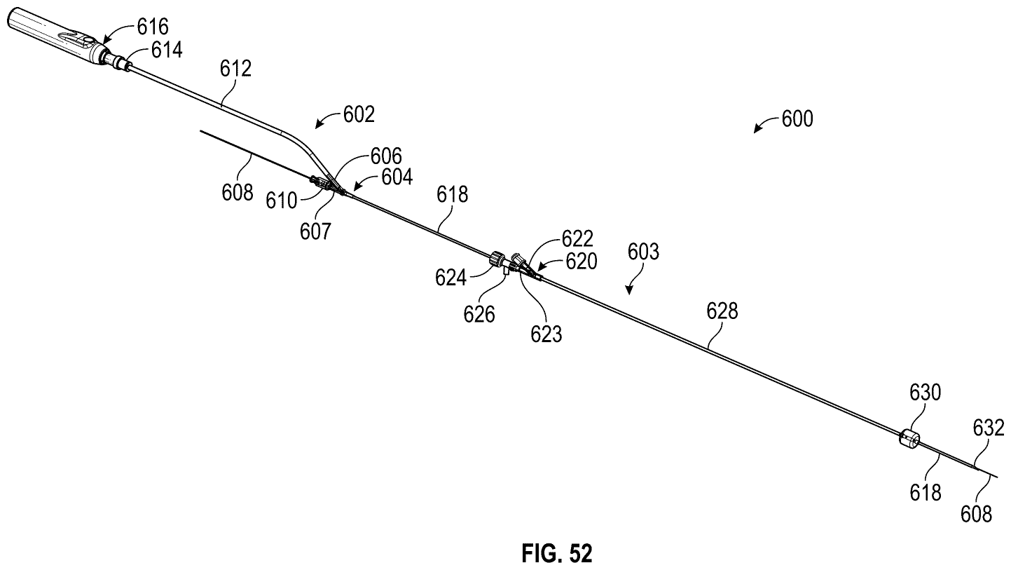

FIG. 52 illustrates a thermal thrombectomy system 600, which can also be referred to as a thermal thrombectomy device, thermal device or system, thermal crossing device or system, crossing device or system, thrombectomy catheter device or system, and/or thermal catheter device or system. The thermal thrombectomy system 600 can be used to cross, which can include penetrating and/or passing through a thrombus or other occlusion within the vasculature system of the body. For example, the thermal thrombectomy system 600 can be used to pass from a proximal side of a thrombus to a distal side. Another device, such as a cutting device and/or collection device, can be introduced to cut and/or collect the thrombus. In some variants, the thermal thrombectomy system 600 can be used to break up (e.g., fragment) the thrombus. The thermal thrombectomy system 600 can be used with any of the other devices and/or systems described herein. The thermal thrombectomy system 600 can include any of the features of other devices and/or systems described herein.

As illustrated, the thermal thrombectomy system 600 can include a thermal assembly 602 (e.g., crossing assembly, thermal crossing assembly) and/or an anchoring assembly 603 (e.g., balloon assembly, expandable assembly). The thermal assembly 602 can be used to penetrate, cross, and/or fragment a thrombus. The anchoring assembly 603 can be used to anchor the thermal thrombectomy system 600 within vasculature to facilitate advancing the thermal assembly 602 distally to penetrate a thrombus. The anchoring assembly 603 can be used to impede the flow of fluid and/or thrombus proximally. The anchoring assembly 603 can be used to center the thermal assembly 602 (e.g., heated element thereof) within vasculature.

The anchoring assembly 603 can include a sheath 628 (e.g., tube, catheter, outer tube). The anchoring assembly 603 can include an expandable device 630 (e.g., balloon, umbrella). The expandable device 630 can be disposed on a distal portion of the sheath 628. The expandable device 630 can be expanded (e.g., inflated) when positioned in vasculature to contact blood vessel walls (e.g., vein or artery walls), which can anchor the anchoring assembly 603 and/or thermal thrombectomy system 600 in place within the blood vessel. The expanded expandable device 630 can impede the proximal flow of fluid, such as blood, and/or thrombus. The expandable device 630, when a balloon, can be filled or emptied of a gas and/or fluid to expand or deflate. The expandable device 630, when an umbrella or the like, can be expanded using shape memory material or other techniques. The expandable device 630 can center the anchoring assembly 603 (e.g., sheath 628) within the vasculature when expanded to contact the vessel walls.

The anchoring assembly 603 can include a connector 620 (e.g., Y coupling, coupler, Y coupler, Y connector). The connector 620 can be disposed at a proximal portion of the sheath 628. The connector 620 can include a port 622 (e.g., angled port, inlet, inflation port). Gas and/or fluid can be introduced or removed through the port 622 of the connector 620 to inflate or deflate the expandable device 630. For example, the gas and/or fluid can flow through the port 622 and interior of the sheath 628 to the expandable device 630 for inflation.

The connector 620 can include a port 626 (e.g., flush port, inlet, flush inlet). Fluid can be introduced into the sheath 628 by way of the port 626 to flush the anchoring assembly 603 of air to reduce the risk that air is introduced into the vasculature.

The connector 620 can include a port 623 (e.g., straight port, main port). The port 623 can receive the thermal assembly 602, guide wire, and/or other devices therethrough.

The connector 620 can include a valve 624 (e.g., Tuohy-Borst valve, adapter, Tuohy-Borst adapter). The valve 624 can seal and/or clamp onto the thermal assembly 602, such as a tube 618 of the thermal assembly 602. The valve 624 can be disposed on the port 623.

The thermal assembly 602 can be introduced through the anchoring assembly 603. For example, the thermal assembly 602 can include the tube 618 (e.g., catheter, outer tube). The tube 618 can be advanced distally through the anchoring assembly 603. For example, the tube 618 can be advanced through the connector 620 (e.g., port 623, valve 624 on the port 623 of the connector 620) and the sheath 628 of the anchoring assembly 603. The tube 618 can be advanced distally through the sheath 628 such that a distal portion of the tube 618 can extend distally out of the sheath 628 (e.g., distal of a distal end of the sheath 628).

The thermal assembly 602 can include a heated element 632 (e.g., electrode, heated wire, heated tube, heated loop, heated member, heated tip, heated end, heated distal portion, heated distal end). The heated element 632 can be disposed on a distal portion of the tube 618 such that advancing the tube 618 distally out of the sheath 628 positions the heated element 632 distal of a distal end of the sheath 628. The heated element 632 can be heated. The heated element 632 can be heated directly or indirectly with one or more energy sources, which may at least include heat, radio frequency, laser, electricity (e.g., current), resistive heating, inductive heating, ultrasound, hot liquid, nuclear, and/or others. The heated element 632 can include a variety of materials, which can at least include shape memory materials (e.g., shape memory alloy, shape memory polymer, Nitinol, nickel-titanium alloy), metals, metal alloys, polymers, ceramics, etc. The heated element 632 can be advanced to contact a thrombus to ease penetration of the thrombus, soften and/or emulsify the thrombus, cross the thrombus, and/or break up (e.g., fragment) the thrombus. The heated element 632 can be used to core the thrombus. The expanded expandable device 630 can anchor the thermal thrombectomy system 600 within the vasculature to facilitate advancing the heated element 632 (e.g., facilitate penetrating the thrombus). Wiring can be routed to the heated element 632 through the interior of the tube 618.

The thermal assembly 602 can include a connector 604 (e.g., Y coupling, coupler, Y coupler, Y connector). The connector 604 can be disposed at a proximal portion of the tube 618. The connector 604 can include a port 606 (e.g., angled port, inlet, inflation port). One or more conductors (e.g., wire(s)) for the thermal thrombectomy system 600 can be routed through the port 606. For example, conduits for heating, temperature sensing, leakage current sensing, and/or mechanical agitators can be routed through the port 606 and into the tube 618.

The thermal assembly 602 can include a handpiece 616 (e.g., controller, handle). The handpiece 616 can include electronics (e.g., battery, rechargeable battery, controller, processor, memory, timer, wireless communication interface, transceiver, power interface, etc.) to facilitate the functionalities of the thermal thrombectomy system 600 described herein. The handpiece 616 can be used to control the temperature of the heated element 632. For example, an electrical current can be applied from a battery of the handpiece 616 to the heated element 632. In some variants, electrical current can be applied from an external power source to the heated element 632. The electronics (e.g., controller) of the handpiece 616 can be used to automatically control the temperature of the heated element 632, which can be based on sensed temperature(s) and/or sensed current leakage. For example, if the sensed temperature is above a threshold, the controller of the handpiece 616 can lower the temperature of the heated element 632 (e.g., adjust electrical current and/or voltage to lower temperature). If the sensed temperature is below a threshold, the controller of the handpiece 616 can raise the temperature of the heated element 632 (e.g., adjust electrical current and/or voltage to raise temperature). If leaked current is sensed, the controller of the handpiece 616 can cease applying a current to the heated element 632.

The thermal assembly 602 can include an electrical connector 614 (e.g., push-pull latching electrical connector). The conduits for heating, temperature sensing, leakage current sensing, and/or mechanical agitators can be connected to the electrical connector 614. The electrical connector 614 can be coupled (e.g., releasably coupled) with the handpiece 616. The conduits can be routed from the electrical connector 614 to the port 606 of the connector 604 by way of a tube 612 (e.g., flexible tube, conduit, flexible conduit). The tube 612 can be insulated.

The connector 604 can include a port 607 (e.g., straight port, main port). A hub 610 (e.g., valve, connector) can be disposed on the port 607.

A guide wire 608 (e.g., wire) can be used to navigate the thermal thrombectomy system 600 to a thrombus. For example, the guide wire 608 can be routed through the vasculature of a patient and the thermal thrombectomy system 600 can be advanced over the guide wire 608. For example, the sheath 628 and/or tube 618 can be advanced over the guide wire 608. The guide wire 608 can extend through the port 607, hub 610, tube 618, port 623, valve 624, and/or sheath 628 to distal of the sheath 628. The hub 610 can seal around guide wire 608, which can impede fluid from escaping.

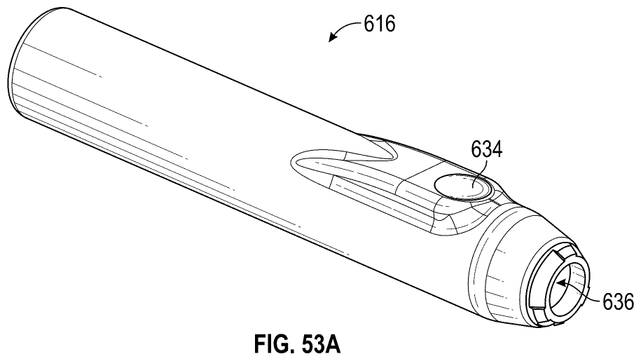

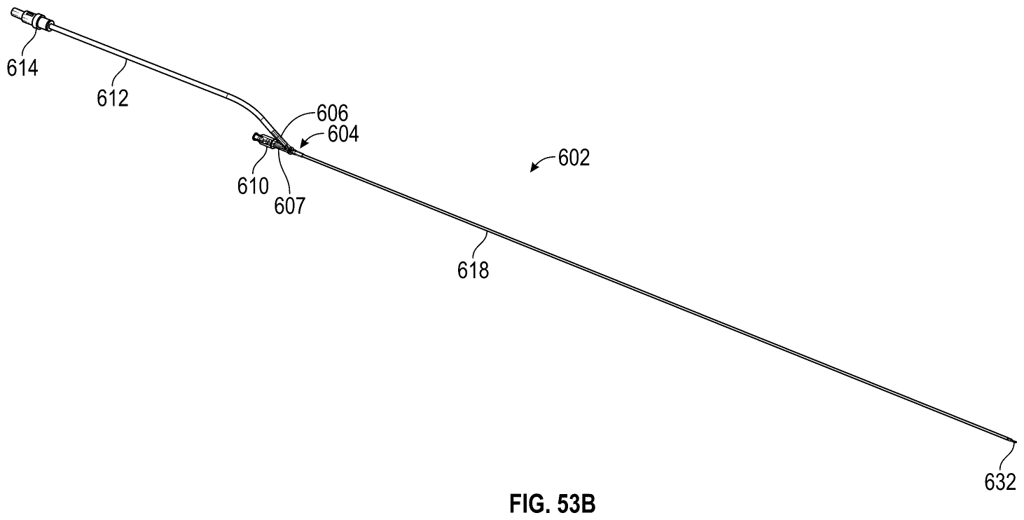

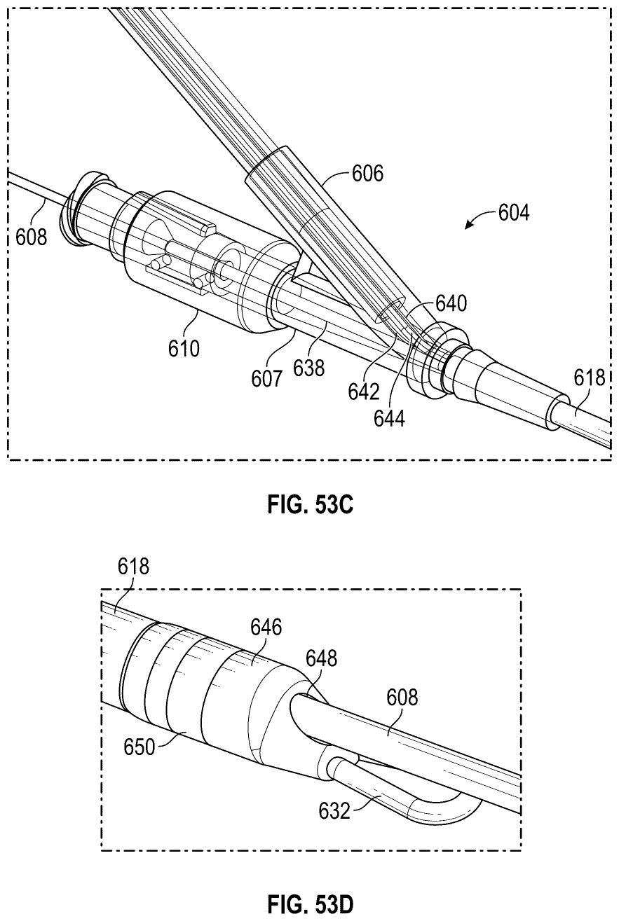

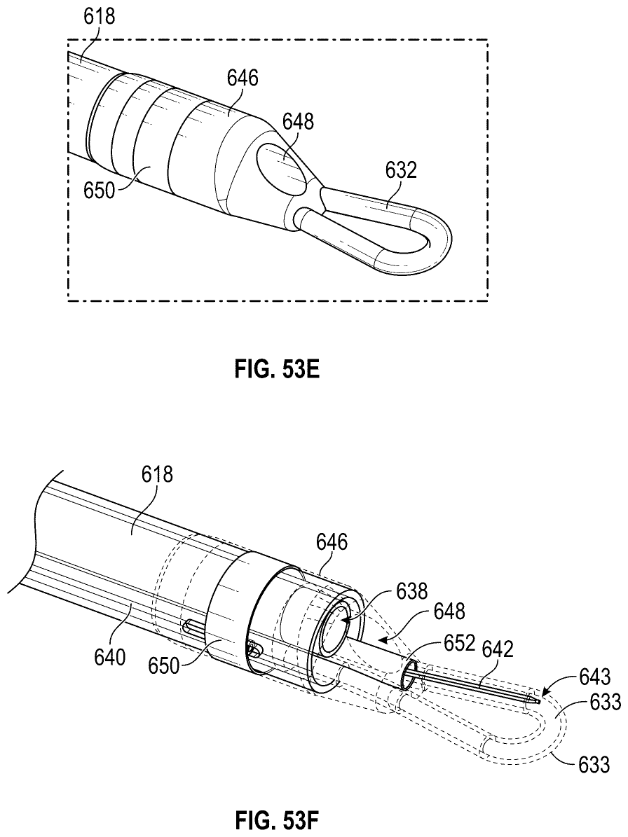

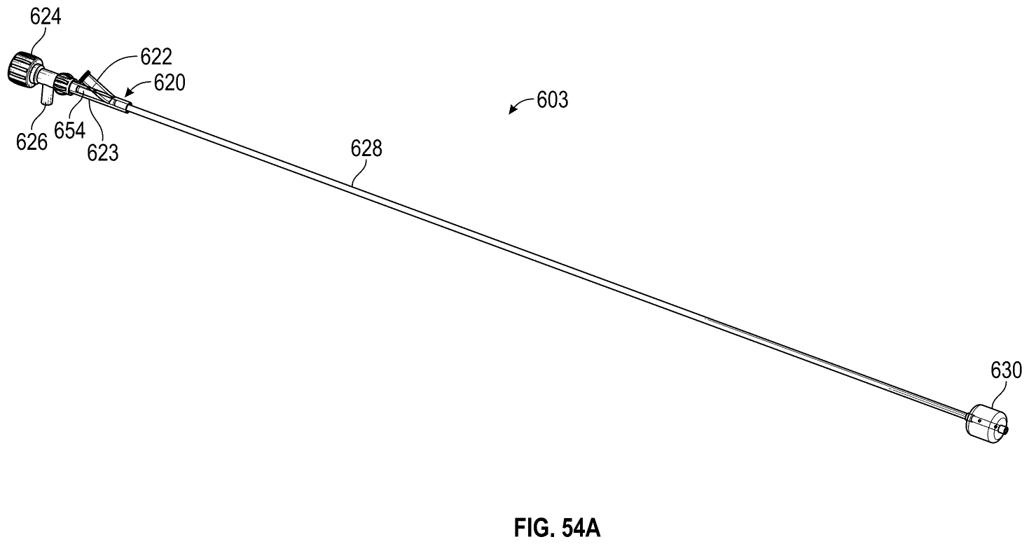

FIG. 53A illustrates the handpiece 616 for the thermal thrombectomy system 600 (e.g., the thermal assembly 602 of the thermal thrombectomy system 600). As shown, the handpiece 616 can include a socket 636 (e.g., connection feature, interface) to couple with the electrical connector 614 to electrically couple with the one or more conduits in the tube 612.

The handpiece 616 can include a button 634 or the like (e.g., toggle, switch, dial, etc.) to activate (e.g., begin heating) the heated element 632. In some embodiments, the handpiece 616 can include a display (e.g., gauge) and/or other indicator that can indicate the sensed temperature at the heated element 632, status (e.g., activated or not) of the heated element 632, if current leakage has been detected, and/or other characteristics of the thermal thrombectomy system 600. In some embodiments, the handpiece 616 can include a user interface (e.g., button, touch screen, toggle, switch, dial) to set a target temperature for the heated element 632.

FIG. 53B illustrates the thermal assembly 602 with the handpiece 616 decoupled from the electrical connector 614, the tube 618 disposed outside of the sheath 628 of the anchoring assembly 603, and the guide wire 608 removed.

FIG. 53C illustrates an enlarged view of the connector 604 of the thermal assembly 602 and components of the thermal thrombectomy system 600 disposed therein. As shown, an electrical leakage conduit 640, temperature sensor conduit(s) 642 (e.g., thermocouple conduits), and/or heat conduit(s) 644 can be routed through the tube 612 to the port 606 of the connector 604. The conduits can be wires. The electrical leakage conduit 640, thermocouple conduit 642, and/or heat conduit(s) 644 can be routed through the port 606 and into the tube 618 (e.g., proximal end of the tube 618). The heat conduit(s) 644 can deliver an electrical current and/or other form of energy to the heated element 632 to raise a temperature of the heated element 632. As shown, the thermal assembly 602 can include a tube 638 (e.g., catheter, inner tube) through which the guide wire 608 can be disposed. The tube 638 can be disposed in the connector 604 (e.g., port 607, hub 610) and extend into the tube 618 (e.g., through a proximal end of the tube 618). In some embodiments, the tube 638 can terminate and/or be sealed in the connector 604 (e.g., port 607, hub 610). In some embodiments, the hub 610 can seal around the tube 638. The tube 618 can terminate in the connector 604 (e.g., distal of the port 606, distal end of connector 604).

FIG. 53D-53F illustrate a distal portion (e.g., crossing tip, heated tip) of the thermal assembly 602. As shown, the thermal assembly 602 can include a distal end 646 (e.g., distal tip), which can be a reflowed. The distal end 646 can include generally rounded features. The distal end 646 can seal (e.g., plug) the distal portion of the tube 618. The distal end 646 can be a plug disposed on the tube 618. The distal end 646 can include an opening 648 (e.g., hole) through which the guide wire 608 can be disposed (e.g., the guide wire 608, thermal assembly 602) can be advanced distally over the guide wire 608 with the guide wire 608 disposed through the opening 648). The distal end 646 can include the heated element 632. The heated element 632 can be a loop (e.g., include a looped shape), which can be a Nitinol loop. The heated element 632 can extend distally from the distal end 646 and curve back proximally to the distal end 646. In some embodiments, the distal end 646 can be formed around proximal portions of the loop of the heated element 632. The heated element 632 can be coated in a parylene coating for insulation (e.g., thin parylene coating).

The thermal assembly 602 can include a feature for fluoroscopy and/or X-ray visualization. The thermal assembly 602 can include one or more features for detecting current leaks from the heated element 632. For example, the thermal assembly 602 can include an electrode and marker, which can be the same feature. The thermal assembly 602 can include an electrode 650, which can be a band, annular structure, and/or ring. The electrode 650 can be disposed around the distal end 646. The electrode 650 can be disposed around the tube 618. The electrode 650 can be used for visualization for navigating through the vasculature, which can at least include fluoroscopy and/or X-ray visualization techniques. The electrode 650 can be used to detect a current escaped from the heated element 632. If current is detected (e.g., current above a threshold is detected), the thermal assembly 602 can cease applying an electrical current to the heated element 632.

As illustrated in FIG. 53F, the electrical leakage conduit 640 can be electrically coupled (e.g., welded, crimped) to the electrode 650. The electrical leakage conduit 640 can be routed from the electrode 650 back to the handpiece 616 through the tube 618. The electrical leakage conduit 640 can communicate current leakage sensed by the electrode 650 back to the electronics of the handpiece 616, which can automatically stop applying a current to the heated element 632.

The thermal assembly 602 can include a temperature sensor 643 (e.g., thermocouple). The temperature sensor 643 can be disposed at the heated element 632 to sense temperature at the heated element 632. The temperature sensor 643 can be disposed inside of the heated element 632. As shown in FIG. 53F, the heated element 632 can include an interior 633 (e.g., hollow interior) in which the temperature sensor 643 can be placed. The thermocouple conduit(s) 642 can be routed from the temperature sensor 643 back through the tube 618, connector 604 (e.g., port 606 of the connector 604), and tube 612 to the handpiece 616 to communicate sensed temperature data.

The thermal assembly 602 can include insulation 652 (e.g., an insulating sleeve) over at least one of the legs of the heated element 632 (e.g., one of the legs of the loop of the heated element 632) which can help reduce the likelihood of shorts between the legs of the loop.

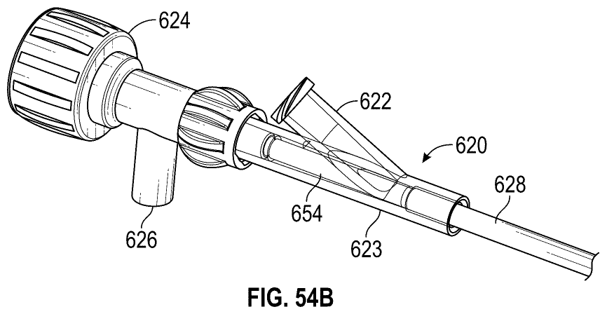

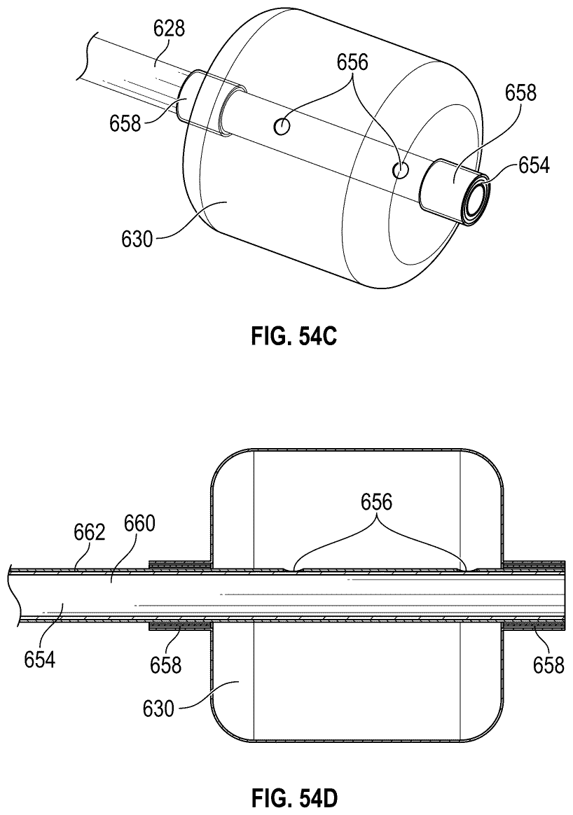

FIG. 54A illustrates the anchoring assembly 603 separate from the thermal assembly 602. In some embodiments, the anchoring assembly 603 (e.g., sheath 628) can be advanced over the guide wire 608 to position the expandable device 630 proximal of a thrombus and then the thermal assembly 602 can be advanced through the sheath 628.

FIG. 54B illustrates a proximal portion of the anchoring assembly 603. As illustrated, the anchoring assembly 603 can include a tube 654 (e.g., catheter, inner balloon tube, inner tube). The proximal end of the tube 654 can terminate and/or be sealed in the connector 620, which can include in the port 623.

As illustrated in FIGS. 54C and 54D, the tube 654 can extend to within a distal portion of the sheath 628. The expandable device 630 (e.g., balloon) can be disposed on the distal portion of the sheath 628. The sheath 628 can include one or more openings 656 (e.g., holes), which can include two holes, through which gas or fluid can flow to inflate or deflate the expandable device 630. The fluid can flow in the gap 662 (e.g., radial gap) between the outer surface of the tube 654 and the inner surface of the sheath 628 to facilitate flow between the openings 656 and the port 622. The sheath 628 can include retainers 658 (e.g., anchors, bands, rings) disposed on either side of the expandable device 630. The retainers 658 can couple the proximal and distal sides of the expandable device 630 to the sheath 628, which can impede fluid and/or gas from escaping out of the expandable device 630 and/or impede the expandable device 630 from sliding along the sheath 628. In some variants, the distal end of the tube 654 can be sealed to the interior of the distal end of the sheath 628 to prevent fluid and/or gas escaping out of the sheath 628. The tube 654 can include a lumen 660 through which the thermal assembly 602 (e.g., tube 618, distal end 646, and/or heated element 632) can be advanced.

To expand the expandable device 630 (e.g., balloon), a fluid and/or gas can be introduced through the port 622. The fluid and/or gas can travel in the gap 662 between the tube 654 and the sheath 628 until flowing into the expandable device 630 by way of the one or more openings 656 to fill the expandable device 630. To deflate the expandable device 630, the fluid and/or gas can flow back through the one or more openings 656, in the gap 662 between the tube 654 and the sheath 628, and out the port 622.

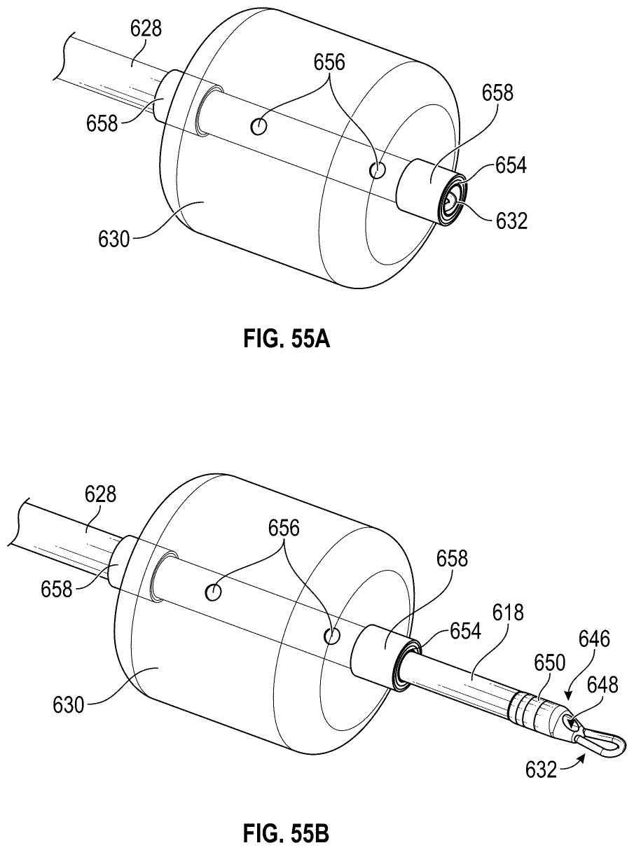

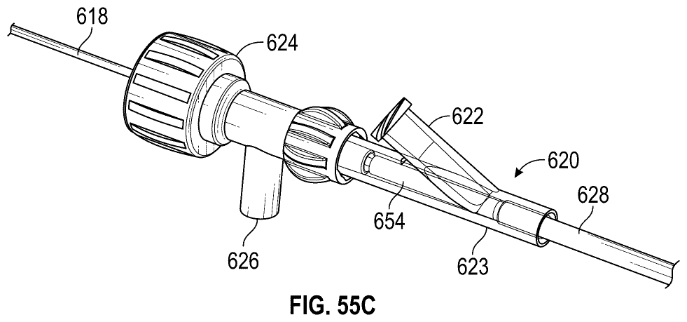

As illustrated in FIGS. 55A and 55B, the thermal assembly 602 can be advanced distally through the tube 654. The thermal assembly 602 can be advanced such that the distal end 646 with the heated element 632 is positioned distally outside of the sheath 628. The exposed heated element 632 can be heated and advanced to contact a thrombus and/or other occlusion. As illustrated in FIG. 55C, the thermal assembly 602 (e.g., tube 618, distal end 646, and/or heated element 632) can be advanced distally through the connector 620 (e.g., port 623 and/or valve 624) and into the tube 654 positioned within the tube 654. The tube 654 can seal and/or clamp onto the tube 618.

In use, the guide wire 608 can be navigated through vasculature to a thrombus. The thermal assembly 602 (e.g., tube 618) can be advanced through the anchoring assembly 603. For example, the thermal assembly 602 (e.g., tube 618) can be advanced distally through the connector 620 (e.g., valve 624, tube 654) and the tube 654 disposed inside the sheath 628, which can include being advanced distally inside the sheath 628 until within a distal portion of the sheath 628 such as shown in FIG. 55A. The valve 624 can be used to clamp onto the tube 618 to couple the thermal assembly 602 and anchoring assembly 603 together. The proximal end of the guide wire 608 can be positioned through the opening 648 of the distal end 646. The thermal assembly 602 and anchoring assembly 603 can be advanced distally together over the guide wire 608 such that the guide wire 608 extends through the tube 638 in the tube 618 of the tube 618 and out the connector 604 (e.g., port 607, hub 610). The electrode 650 disposed on the distal end 646 of the tube 618 can help visually locate the thermal thrombectomy system 600 in the vasculature as the thermal assembly 602 and anchoring assembly 603 are distally advanced over the guide wire 608. In some variants, the anchoring assembly 603 can be distally advanced over the guide wire 608 followed by the thermal assembly.

With the distal portions of the thermal assembly 602 and anchoring assembly 603 disposed proximate and proximally of a thrombus and/or other occlusion, the expandable device 630 can be expanded. Fluid and/or gas can be introduced through the port 622 to inflate the expandable device 630 as described herein. The expandable device 630 can expand to contact the surrounding vessel walls to anchor and/or center the anchoring assembly 603. The valve 624 can be unclamped from the tube 618 of the thermal assembly 602. The thermal assembly 602 can be advanced such that the heated element 632 is disposed distally out of the tube 654 and/or sheath 628. The clinician can initiate heating of the heated element 632 with the handpiece 616 (e.g., pushing the button 634). The heated element 632 can be heated to a temperature, which can at least include any of those described herein. The temperature sensor 643 can sense a temperature of the heated element 632, which can be used to automatically control the temperature of the heated element 632 (e.g., adjust current and/or voltage).

The heated element 632 can be advanced to contact the thrombus. The heat from the heated element 632 can ease penetration of the thrombus, which can include softening and/or emulsifying the thrombus (e.g., softening and/or emulsifying the core of the thrombus). In some variants, the heated element 632 can penetrate and cross over the thrombus without assistance from the guide wire 608. In some variants, the heated element 632 and guide wire 608 can cooperate to penetrate and/or cross the thrombus. For example, the heated element 632 may be advanced to contact the thrombus and apply heat to soften and/or emulsify the thrombus and then the guide wire 608 may be advanced to engage the thrombus.

With the heated element 632 and guide wire 608 crossed over the thrombus (e.g., positioned distally of the thrombus), the thermal assembly 602 can be retracted out of the vasculature through the lumen 660 of the tube 654 disposed inside the sheath 628. A capture device and/or cutting device can be advanced distally over the guide wire 608 and through the sheath 628 (e.g., tube 654 disposed inside the sheath 628). The capture device, which can at least include any of those described herein, can be disposed distally of the thrombus. The capture device can be expanded distally of the thrombus and retracted to capture and remove the thrombus, which can include removal through the lumen 660 of the tube 654 in the sheath 628. In some variants, a cutting device, which may include heated elements, can be introduced through the anchoring assembly 603 to fragment the thrombus for removal. The anchoring assembly 603 and/or guide wire 608 can be retracted proximally for removal.

The actions, steps, methods, etc. described herein can be performed by a clinician (e.g. surgeon) and/or robot.

### Terminology

Although the systems and methods have been disclosed in the context of certain embodiments and examples, it will be understood by those skilled in the art that the systems and methods extend beyond the specifically disclosed embodiments to other alternative embodiments and/or uses of the embodiments and certain modifications and equivalents thereof. Various features and aspects of the disclosed embodiments can be combined with or substituted for one another in order to form varying modes of the systems and methods. The scope of this disclosure should not be limited by the particular disclosed embodiments described herein.

Methods of using the foregoing system(s) (including device(s), apparatus(es), assembly(ies), structure(s) or the like) are included; the methods of use can include using or assembling any one or more of the features disclosed herein to achieve functions and/or features of the system(s) as discussed in this disclosure. Methods of manufacturing the foregoing system(s) are included; the methods of manufacture can include providing, making, connecting, assembling, and/or installing any one or more of the features of the system(s) disclosed herein to achieve functions and/or features of the system(s) as discussed in this disclosure.

Certain features that are described in this disclosure in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations, one or more features from a claimed combination can, in some cases, be excised from the combination, and the combination may be claimed as any subcombination or variation of any subcombination.

Moreover, while operations may be depicted in the drawings or described in the specification in a particular order, such operations need not be performed in the particular order shown or in sequential order, and all operations need not be performed, to achieve the desirable results. Other operations that are not depicted or described can be incorporated in the example methods and processes. For example, one or more additional operations can be performed before, after, simultaneously, or between any of the described operations. Further, the operations may be rearranged or reordered in other implementations. Also, the separation of various system components in the implementations described above should not be understood as requiring such separation in all implementations, and it should be understood that the described components and systems can generally be integrated together in a single product or packaged into multiple products. Additionally, other implementations are within the scope of this disclosure.

Conditional language, such as “can,” “could,” “might,” or “may,” unless specifically stated otherwise, or otherwise understood within the context as used, is generally intended to convey that certain embodiments include or do not include, certain features, elements, and/or steps. Thus, such conditional language is not generally intended to imply that features, elements, and/or steps are in any way required for one or more embodiments.

Conjunctive language, such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Some embodiments have been described in connection with the accompanying drawings. Components can be added, removed, and/or rearranged. Orientation references such as, for example, “top” and “bottom” are for ease of ease of discussion and may be rearranged such that top features are proximate the bottom and bottom features are proximate the top. Further, the disclosure herein of any particular feature, aspect, method, property, characteristic, quality, attribute, element, or the like in connection with various embodiments can be used in all other embodiments set forth herein. Additionally, it will be recognized that any methods described herein may be practiced using any device suitable for performing the recited steps.

In summary, various embodiments and examples of thermal thrombectomy devices, systems, and methods have been disclosed. Although the systems and methods have been disclosed in the context of those embodiments and examples, it will be understood by those skilled in the art that this disclosure extends beyond the specifically disclosed embodiments to other alternative embodiments and/or other uses of the embodiments, as well as to certain modifications and equivalents thereof. This disclosure expressly contemplates that various features and aspects of the disclosed embodiments can be combined with, or substituted for, one another. Accordingly, the scope of this disclosure should not be limited by the particular disclosed embodiments described above, but should be determined only by a fair reading of the claims that follow.

What is claimed is:

1. A thermal device configured to apply heat to a thrombus, the thermal device comprising:
an outer tube comprising a distal portion;
a heated element disposed at the distal portion of the outer tube; and
one or more conduits configured to apply an electrical current to the heated element to raise a temperature of the heated element to apply heat to a thrombus.

2. The thermal device of claim 1, further comprising an inner tube disposed within the outer tube that is configured to receive a guide wire, wherein the thermal device is configured to be advanced over the guide wire.

3. The thermal device of claim 1, wherein the heated element comprises a loop.

4. The thermal device of claim 3, wherein the loop comprises a nickel titanium alloy.

5. The thermal device of claim 3, wherein the loop comprises a tube.

6. The thermal device of claim 5, wherein the loop comprises a hollow lumen, and the thermal device further comprising a temperature sensor disposed within the hollow lumen.

7. The thermal device of claim 6, further comprising a controller configured to modulate the electrical current applied to the heated element based on a sensed temperature by the temperature sensor.

8. The thermal device of claim 1, further comprising a temperature sensor.

9. The thermal device of claim 1, further comprising an electrode to sense leaked current.

10. The thermal device of claim 9, wherein the electrode is a marker for visualization.

11. The thermal device of claim 1, further comprising an expandable device configured to be expanded proximally of the thrombus.

12. The thermal device of claim 11, wherein the expandable device comprises a balloon.

13. The thermal device of claim 11, wherein the expandable device comprises a lumen through which the outer tube is configured to be advanced.

14. A thermal thrombectomy device configured to apply heat to a thrombus, the thermal thrombectomy device comprising:
an expandable assembly comprising an expandable device, the expandable device configured to be expanded proximally of a thrombus; and
a thermal assembly comprising a heated element, the thermal assembly configured to be advanced distally over a guide wire and out of the expandable assembly to apply heat to the thrombus with the heated element.

15. The thermal thrombectomy device of claim 14, wherein the thermal assembly further comprises one or more conduits configured to apply an electrical current to the heated element to raise a temperature of the heated element.

16. The thermal thrombectomy device of claim 14, wherein the expandable device is a balloon.

17. The thermal thrombectomy device of claim 14, wherein the heated element comprises a loop.

18. The thermal thrombectomy device of claim 17, wherein the loop comprises a tube.

19. The thermal thrombectomy device of claim 18, wherein the loop comprises a hollow lumen, and the thermal thrombectomy device further comprises a temperature sensor disposed within the hollow lumen.

20. The thermal thrombectomy device of claim 19, further comprising a controller configured to modulate electrical current applied to the heated element based on a sensed temperature by the temperature sensor.

21. The thermal thrombectomy device of claim 14, further comprising a temperature sensor.

22. The thermal thrombectomy device of claim 14, further comprising an electrode to sense leaked current.

23. A method of crossing a thrombus, the method comprising:
advancing a thermal assembly over a guide wire to a thrombus; and
applying an electrical current to a heated element of the thermal assembly to heat the thrombus.

24. The method of claim 23, further comprising advancing an expandable device comprising a balloon over the guide wire to proximate the thrombus and expanding the balloon.
